(19) United States
(12) Patent Application Publication
(10) Pub. No.: US 2026/0306622 A1
(43) Pub. Date: Oct. 8, 2026

(54) TREATMENT OF PEANUT ALLERGY WITH TOLERIZING NANOPARTICLES

(71) Applicant: COUR PHARMACEUTICALS DEVELOPMENT COMPANY INC., Skokie, IL (US)

(72) Inventors: John Puisis, Northbrook, IL (US); Michael Boyne, Skokie, IL (US); Adam Elhofy, Chicago, IL (US); Joseph Podojil, Northbrook, IL (US); Greta Wodarcyk, Northbrook, IL (US)

(21) Appl. No.: 19/121,245

(22) PCT Filed: Oct. 19, 2023
(86) PCT No.: PCT/US23/77287

§ 371 Date: Apr. 15, 2025

Related U.S. Application Data
(60) Provisional application No. 63/380,173, filed on Oct. 19, 2022.
(60) Provisional application No. 63/486,812, filed on Feb. 24, 2023.

(51) Int. Cl. A61K 39/35; A61K 9/00; A61K 9/19; A61K 31/56; A61K 39/00; A61K 45/06; A61P 37/08

(52) U.S. Cl. CPC A61K 39/35; A61K 9/0019; A61K 9/19; A61K 31/56; A61K 45/06; A61P 37/08; A61K 2039/54; A61K 2039/545; A61K 2039/55555; A61K 2039/577

(57) ABSTRACT

The present disclosure relates, in general to methods of treating peanut allergy using nanoparticles encapsulating peanut antigens to induce antigen-specific tolerance.

### CROSS-REFERENCE TO RELATED APPLICATIONS

The present application claims the priority benefit of U.S. Provisional Patent Application No. 63/380,173, filed Oct. 19, 2022, and U.S. Provisional Patent Application No. 63/486,812, filed Feb. 24, 2023, herein incorporated by reference in their entireties.

### FIELD OF THE DISCLOSURE

The present disclosure relates to the methods of treating peanut allergy using tolerizing immune modifying nanoparticles encapsulating purified peanut extract containing allergenic peanut proteins or antigenic fragments thereof.

### DESCRIPTION OF THE TEXT FILE SUBMITTED ELECTRONICALLY

The contents of the text file submitted electronically herewith are incorporated herein by reference in their entirety: A computer readable format copy of the sequence listing (Filename: 58512_SeqListing.XML, Created: Oct. 18, 2023, File Size 27,434 Bytes).

### BACKGROUND

Peanut allergy is one of the most common food allergies affecting nearly 1.2% of the total US population and 2.5% of the pediatric population with incidence rates on the rise over the past decade (Cannon H E. Am J Manag Care. 2018; 24(19 Suppl):S428-s433). Peanut allergy is driven by a pathologic hyperimmune response where exposure to peanut can lead to mild to severe symptoms such as nausea, vomiting, rashes, impaired breathing, drop in blood pressure, and even death.

Normal individuals with a healthy immune system are able to maintain non-responsiveness to antigens encountered in common food substances like peanut; however, in peanut allergic subjects, the loss of immune tolerance to peanut antigens drives the pathologic hyperimmune response. To date, 8 peanut proteins (Ara h1 to Ara h8) have been identified as the predominant antigenic peanut proteins driving the allergic hyperimmune response (Keet et al., J Allergy Clin Immunol Pract. 2013; 1(1):101-103). Additional peanut allergens have also been described recently reporting up to a total of 18 allergenic peanut proteins. Ozias-Akins et al., Allergy. 2019 May; 74(5): 888-898). The allergic immune response to peanut antigenic proteins is mediated by a T cell dependent mechanism involving the upregulation T helper type-2 (Th2) cytokine production (e.g., IL-4, IL-5, IL-9, and IL-13,) and B cell class switching leading to the production of IgE antibody and degranulation of mast cells and basophils (Sampath et al., J Clin Invest. 2019; 129(4):1431-1440).

Currently, there is no cure for peanut allergy with strict avoidance of exposure to peanut antigens and management of anaphylaxis the only options available to patients. Immune tolerizing therapies which can induce T-cell tolerance to peanut antigens are considered the gold standard for the treatment of peanut allergy but have been elusive thus far. Several attempts at developing immune tolerizing therapies have been made using oral immunotherapy (OIT), subcutaneous immunotherapy (SCIT), epicutaneous immunotherapy (EPIT), and sublingual immunotherapy (SLIT) approaches (Feuille et al., Allergy Asthma Immunol Res. 2018; 10(3):189-206). The success of these therapies has been highly variable and only desensitization to peanut proteins has been reported which offers protection against only accidental exposure but is not a cure (Chinthrajah et al., Lancet. 2019; 394(10207):1437-1449; Vickery et al., N Engl J Med. 2018; 379(21):1991-2001; Fleischer et al., J. Am Med Assoc. 2019; 321(10):946-955).

### SUMMARY

Tolerizing immune modifying particles (TIMPs), comprising one or more antigens, have been previously described for the treatment of immune-mediated disorders (e.g., autoimmune diseases and allergies) via induction of antigen-specific immune tolerance (WO20131319253 and WO2015023796 incorporated herein by reference). In several preclinical models of autoimmune diseases and allergies, TIMPs have demonstrated efficacy at inducing T-cell tolerance. Induction of antigen-specific T cell tolerance to peanut antigens using TIMPs encapsulating purified peanut extract (PPE) and/or comprising a peanut extract, or one or more peanut proteins or antigenic fragments thereof, including Ara h1, Ara h2, Ara h3, Ara h5, Ara h6, Ara h7, Ara h8, Ara h9, Ara h10, Ara h11, Ara h12, Ara h13, Ara h14, Ara h15, Ara h16, Ara h17, and Ara h18 (TIMP-PPE) could ameliorate or potentially cure peanut allergy (PA).

Provided herein is a method of treating peanut allergy in a subject comprising administering to the subject TIMP-PPE, wherein TIMP-PPE is administered at a dose of 0.001 to 12 mg/kg. Also provided herein is a method of reducing an allergic immune response to peanut antigens in a subject suffering from PA comprising administering to the subject TIMP-PPE, wherein TIMP-PPE is administered at a dose of about 0.001 to 12 mg/kg. In various embodiments, the TIMP-PPE is administered at a dose from about from about 0.001 to 10 mg/kg, from about 0.005 to 12 mg/kg, from about 0.01 to 12 mg/kg, from about 0.05 to 12 mg/kg, from about 0.1 to 12 mg/kg, from about 0.5 to 10 mg/kg, from about 1 to 8 mg/kg, from about 1.5 to 10 mg/kg, from about 2 to 12 mg/kg, from about 2 to 10 mg/kg, from about 3 to 10 mg/kg, from about 4 to 10 mg/kg, from about 4 to 12 mg/kg, or from about 5 to 12 mg/kg. In various embodiments, TIMP-PPE is administered at a dose of about 0.001 mg/kg, 0.0025 mg/kg, 0.005 mg/kg, 0.01 mg/kg, 0.025 mg/kg, 0.05 mg/kg, 0.1 mg/kg, 0.25 mg/kg, 0.5 mg/kg, 1.0 mg/kg, 2.0 mg/kg, 3.0 mg/kg, 4.0 mg/kg, 5 mg/kg, 6 mg/kg, 8.0 mg/kg, 10 mg/kg, or 12 mg/kg. In various embodiments, TIMP-PPE is administered at a dose of about 0.1 mg, 0.25 mg, 0.5 mg, 1 mg, 2 mg, 2.5 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg.

In various embodiments, TIMP-PPE is administered at a concentration of between about 0.0005 mg/mL and about 50 mg/mL. In various embodiments, TIMP-PPE is administered at a concentration of about 0.0005 mg/mL, 0.001 mg/mL, 0.005 mg/mL, 0.01 mg/mL, 0.05 mg/mL, 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL. In various embodiments, TIMP-PPE is administered via intravenous infusion lasting about 0.5, 1, 2, 3, 4, 5, 6, 7, 8, 10, 12, 18, or 20 hours.

In various embodiments, TIMP-PPE is administered in a single dose or in multiple doses. In various embodiments, TIMP-PPE is administered in two doses one-week apart. In various embodiments, TIMP-PPE is administered once weekly, once every two weeks, once every three weeks, once every 4 weeks, once every two months, once every three months, once every 6 months, or once per year.

In various embodiments, a booster dose of TIMP-PPE is administered, i.e., the TIMP-PPE is re-administered, in a single dose or in multiple doses following the original or initial TIMP-PPE administration. In various embodiments, the booster dose of TIMP-PPE is administered once weekly, once every two weeks, once every three weeks, once every 4 weeks, once every two months, once every three months, once every 6 months, or once per year. In various embodiments, TIMP-PPE is administered in two doses one-week apart followed by a booster dose of TIMP-PPE re-administered as a single dose once every three months.

In various embodiments, booster dose of TIMP-PPE is re-administered at a dose of 0.001 mg/kg to 12 mg/kg. In various embodiments, the booster dose of TIMP-PPE is re-administered at a dose from about 0.001 to 10 mg/kg, from about 0.005 to 12 mg/kg, from about 0.01 to 12 mg/kg, from about 0.05 to 12 mg/kg, from about 0.1 to 12 mg/kg, from about 0.5 to 10 mg/kg, from about 1 to 8 mg/kg, from about 1.5 to 10 mg/kg, from about 2 to 12 mg/kg, from about 2 to 10 mg/kg, from about 3 to 10 mg/kg, from about 4 to 10 mg/kg, from about 4 to 12 mg/kg, or from about 5 to 12 mg/kg, or about 0.001 mg/kg, 0.0025 mg/kg, 0.005 mg/kg, 0.01 mg/kg, 0.025 mg/kg, 0.05 mg/kg, 0.1 mg/kg, 0.25, 0.5 mg/kg, 1.0 mg/kg, 2.0 mg/kg, 4.0 mg/kg, 6 mg/kg, 8.0 mg/kg, 10 mg/kg, or 12 mg/kg. In various embodiments, the booster dose of TIMP-PPE is re-administered at a dose of about 0.1 mg, 0.25 mg, 0.5 mg, 1 mg, 2 mg, 2.5 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg. In various embodiments, the booster dose of TIMP-PPE is administered at a concentration of between about 0.0005 mg/mL and about 50 mg/mL. In various embodiments, the booster dose of TIMP-PPE is re-administered at a concentration of about 0.0005 mg/mL, 0.001 mg/mL, 0.005 mg/mL, 0.01 mg/mL, 0.05 mg/mL, 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL.

In various embodiments, TIMP-PPE booster dose is administered via intravenous infusion lasting about 0.5, 1, 2, 3, 4, 5, 6, 7, 8, 10, 12, 18, or 20 hours.

In various embodiments, TIMP-PPE encapsulates peanut proteins and or antigenic fragments of peanut proteins Ara h1, Ara h2, Ara h3, Ara h5, Ara h6, Ara h7, Ara h8, Ara h9, Ara h10, Ara h11, Ara h12, Ara h13, Ara h14, Ara h15, Ara h16, Ara h17, or Ara h18. In various embodiments, the TIMP-PPE encapsulates peanut proteins and or antigenic fragments of peanut proteins Ara h1, Ara h2, Ara h3, Ara h5, Ara h6, Ara h7, Ara h8, Ara h9, Ara h10, Ara h11, Ara h12, Ara h13, Ara h14, Ara h15, Ara h16, Ara h17, Ara h18, or combinations thereof. In various embodiments, the TIMP-PPE comprises a peanut extract or one or more peanut proteins or antigenic fragments thereof selected from the group consisting of Ara h1, Ara h2, Ara h3, Ara h5, Ara h6, Ara h7, Ara h8, Ara h9, Ara h10, Ara h11, Ara h12, Ara h13, Ara h14, Ara h15, Ara h16, Ara h17, and Ara h18.

In various embodiments, TIMP-PPE consists of poly (lactic co-glycolic acid) (PLGA) particles encapsulating one or more peanut antigens and a suitable buffering agent or excipient. In various embodiments, TIMP-PPE particles are surface functionalized. In various embodiments, TIMP-PPE particles are surface functionalized by carboxylation. In various embodiments, TIMP-PPE particles have a negative zeta potential. In various embodiments, the negative zeta potential of TIMP-PPE particles is between about −100 mV to about 0 mV. In various embodiments, the zeta potential of the particles is from about −100 mV to about −25 mV, from about −100 to about −30 mV, from about −80 mV to about −30 mV, from about −75 mV to about −30 mV, from about −70 mV to about −30 mV, from about −75 to about −35 mV, from about −70 to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV.

In various embodiments, the size, or diameter, of TIMP-PPE particles is between 0.05 μm to about 10 μm. In various embodiments, the diameter of TIMP-PPE particles is between 0.1 μm and about 10 μm. In various embodiments, the diameter of TIMP-PPE particles is between 0.1 μm and about 5 μm. In various embodiments, the diameter of TIMP-PPE particles is between 0.1 μm and about 3 μm. In various embodiments, the diameter of TIMP-PPE particles is between 0.3 μm and about 5 μm. In various embodiments, the diameter of TIMP-PPE particles is about 0.3 μm to about 3 μm. In various embodiments, the diameter of TIMP-PPE particles is between about 0.3 μm to about 1 μm. In various embodiments, the diameter of TIMP-PPE particles is between about 0.4 μm to about 1 μm. In various embodiments, the TIMP-PPE particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-PPE particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the diameter of the negatively charged particle is between 400 nm to 800 nm.

In various embodiments, TIMP-PPE is administered intravenously, subcutaneously, intramuscularly, intraperitoneally, intranasally, or orally.

In various embodiments, TIMP-PPE is administered alone or in combination with one or more additional therapeutics. In various embodiments, the disclosure provides a method of treating peanut allergy or peanut allergy related symptoms in a subject in need thereof comprising administering to the subject a composition comprising TIMP-PPE alone or in combination with a therapeutic useful to treat peanut allergy.

In various embodiments, the therapeutic useful to treat peanut allergy induces regulatory T-cells (Tregs). In various embodiments, the therapeutic useful to treat peanut allergy increases the frequency and/or the number of Tregs. In various embodiments, the therapeutic useful to treat peanut allergy is IL-2 therapy to induce Tregs. In various embodiments, the IL-2 therapy is low dose IL-2, IL-2 muteins engineered to expand Tregs, IL-2 variants engineered to expand Tregs, IL-2 molecules engineered to be selective for the high-affinity IL-2 receptor, PEGylated IL-2, IL-2 complexes, or IL-2/CD25 fusion proteins. In various embodiments, the additional therapeutic is an inhibitor of IgE, an inhibitor of basophil activation, an inhibitor of mast cell activation, an antihistamine, or a small molecule or biological therapeutic. In various embodiments, the additional therapeutic is an inhibitor of IgE, a competitor of IgE for allergen binding sites, an inhibitor of basophil activation, an inhibitor of mast cell activation, an antihistamine, a cytokine inhibitor, microbiome therapy, a small molecule or biological therapeutic. In various embodiments, the additional therapeutic inhibits IgE. In various embodiments, the additional therapeutic inhibits IgE antibodies. In various embodiments, the additional therapeutic inhibits basophil activation. In various embodiments, the additional therapeutic inhibits mast cell activation. In various embodiments, the additional therapeutic is a biologic or a small molecule. In various embodiments, the additional therapeutic is an anti-IgE antibody, an anti-IL-4Rα antibody, an anti-IL13 antibody, an anti-IL-33 antibody, an antihistamine, a steroid, a corticosteroid, a leukotriene modifier, or a nonsteroid anti-inflammatory drug (NSAID).

In various embodiments, the therapeutic useful to treat peanut allergy is an inhibitor of IgE, a competitor of IgE for allergen binding sites, an inhibitor of basophil activation, an inhibitor of mast cell activation, an antihistamine, a cytokine inhibitor, microbiome therapy, a small molecule or biological therapeutic. In various embodiments, the therapeutic useful to treat peanut allergy inhibits IgE. In various embodiments, the therapeutic useful to treat peanut allergy inhibits IgE antibodies. In various embodiments, the therapeutic useful to treat peanut allergy inhibits basophil activation. In various embodiments, the therapeutic useful to treat peanut allergy inhibits mast cell activation. In various embodiments, the therapeutic useful to treat peanut allergy is a biologic or a small molecule. In various embodiments, the therapeutic useful to treat peanut allergy is an anti-IgE antibody, an anti-IL-4Rα antibody, an anti-IL13 antibody, an anti-IL-33 antibody, an antihistamine, a steroid, a corticosteroid, a leukotriene modifier, or a nonsteroid anti-inflammatory drug (NSAID).

In various embodiments, the additional therapeutic is an antihistamine. In various embodiments, the therapeutic useful to treat peanut allergy is an antihistamine. In various embodiments, the antihistamine is a first generation antihistamine. In various embodiments, the antihistamine is a second generation antihistamine. In various embodiments, the antihistamines are selected from the group consisting of brompheniramine, carbinoxamine maleate, chlorpheniramine, clemastine, diphenhydramine, hydroxyzine, triprolidine, azelastine, cetirizine, desloratadine, fexofenadine, levocetrizine, doxylamine, ebastine, embramine, epinephrine, fexofenadine, loratadine, and olopatadine.

In various embodiments, the therapeutic administered in combination with TIMP-PPE is an anti-IgE antibody, anti-IL-4Rα antibody, anti-IL13 antibody, an anti-IL-33 antibody, an antihistamine, a steroid, a corticosteroid, a leukotriene modifier, low dose IL-2, IL-2 muteins engineered to expand Tregs, IL-2 variants engineered to expand Tregs, IL-2 molecules engineered to be selective for the high-affinity IL-2 receptor, PEGylated IL-2, IL-2 complexes, IL-2/CD25 fusion proteins, a prebiotic, a probiotic, histone deacetylase inhibitor, short chain fatty acids (e.g. acetate, butyrate, propionate, butyrate polymer), an inhibitor of IgE, competitor of IgE for allergen binding sites, an inhibitor of basophil activation, an inhibitor of mast cell activation, a cytokine inhibitor, microbiome therapy, a small molecule or biological therapeutic, or a nonsteroid anti-inflammatory drug (NSAID).

In various embodiments, the additional therapeutic is a steroid. In various embodiments, the therapeutic useful to treat peanut allergy is a steroid. In various embodiments, the steroid is selected from the group consisting of beclomethasone, ciclesonide, fluticasone furoate, mometasone, budenoside, fluticasone, triamcinolone, and loteprednol.

In various embodiments, the additional therapeutic is a corticosteroid. In various embodiments, the therapeutic useful to treat peanut allergy is a corticosteroid. In various embodiments, the corticosteroid is selected from the group consisting of cortisone, prednisone, prednisolone, methylprednisolone, dexamethasone, betamethasone, and hydrocortisone.

In various embodiments, the additional therapeutic is a nonsteroid anti-inflammatory drug (NSAID). In various embodiments, the therapeutic useful to treat peanut allergy is a nonsteroid anti-inflammatory drug (NSAID). In various embodiments the NSAID is a non-selective NSAID. In various embodiments the NSAID is a COX-2 selective NSAID. In various embodiments the NSAID is a COX-1 selective NSAID. In various embodiments the NSAID is a prostaglandin synthase inhibitor. In various embodiments, the NSAID is selected from the group consisting diclofenac, diclofenac potassium, diclofenac sodium, diflunisal, etodolac, flurbiprofen, fenoprofen, fenoprofen calcium, ketorolac, ketorolac tromethamine, ketoprofen, tolmetin, tolmetin sodium, acetylsalicylic acid, aspirin, ibuprofen, naproxen, indomethacin, indomethacin sodium, sulindac, felbinac, piroxicam, mefenamic acid, meclofenamate sodium, meloxicam, nabumetone, oxaprozin, piroxicam, celecoxib, etodolac, etoricoxib, lumiracoxib, rofecoxib, and valdecoxib.

In various embodiments, the additional therapeutic is a leukotriene modifier. In various embodiments, the therapeutic useful to treat peanut allergy is a leukotriene modifier. In various embodiments the leukotriene modifier is an antileukotriene. In various embodiments the leukotriene modifier is a leukotriene receptor antagonist. In various embodiments the leukotriene modifier is a leukotriene synthesis inhibitor. In various embodiments the leukotriene modifier is selected from the group consisting of montelukast, zileuton, and zafirlukast.

In various embodiments, the biologic is an antibody. In various embodiments, the antibody is an anti-IgE, anti-IL-4Rα, anti-IL-13, or an anti-IL-33 antibody. In various embodiments, the anti-IgE antibody is omalizumab (XOLAIR®). In various embodiments, the anti-IL-4Rα antibody is dupilumab (DUPIXENT®). In various embodiments, the anti-IL-33 antibody is etokinumab. In various embodiments, the additional therapeutic administered prior to, during, or after the administration of TIMP-PPE. In various embodiments, the additional therapeutic is administered intravenously, subcutaneously, intramuscularly, intraperitoneally, intranasally, or orally.

In various embodiments, the therapeutic useful to treat peanut allergy is administered prior to, during, or after the administration of TIMP-PPE. In various embodiments, the therapeutic useful to treat peanut allergy is administered intravenously, subcutaneously, intramuscularly, intraperitoneally, intranasally, or orally.

In various embodiments, the therapeutic is administered prior to, concomitantly with, or subsequent to the administration of TIMP-PPE. In various embodiments, the therapeutic is administered 1, 2, 3, 4, 5, 6, or 7 days prior to administration of TIMP-PPE. In various embodiments, the therapeutic is administered 1, 2, 3, or 4 weeks prior to administration of TIMP-PPE. In various embodiments, the therapeutic is administered 1, 2, 3, 4, 5, 6, 7, 8, 9, 10, 11, or 12 months prior to administration of TIMP-PPE. In various embodiments, the therapeutic is administered 1, 2, 3, 4, 5, 6, 7, 8, 9, 10, 11, or 12 years prior to administration of TIMP-PPE. In various embodiments, the therapeutic is administered 1, 2, 3, 4, 5, 6, or 7 days subsequent to administration of TIMP-PPE. In various embodiments, the therapeutic is administered 1, 2, 3, or 4 weeks subsequent to administration of TIMP-PPE. In various embodiments, the therapeutic is administered 1, 2, 3, 4, 5, 6, 7, 8, 9, 10, 11, or 12 months subsequent to administration of TIMP-PPE. In various embodiments, the therapeutic is administered 1, 2, 3, 4, 5, 6, 7, 8, 9, 10, 11, or 12 years subsequent to administration of TIMP-PPE.

The present disclosure describes a method of treating Peanut Allergy in a subject comprising administering to the subject TIMP-PPE in combination with an anti-IgE antibody, wherein TIMP-PPE is administered at a dose of about 0.001 to 12 mg/kg, and wherein the anti-IgE antibody is administered at a dose of about 10 mg to about 500 mg. In various embodiments, the anti-IgE antibody is omalizumab (XOLAIR®). In various embodiments, the TIMP-PPE is administered at a dose from about 0.001 to 10 mg/kg, from about 0.005 to 12 mg/kg, from about 0.01 to 12 mg/kg, from about 0.05 to 12 mg/kg, from about 0.1 to 12 mg/kg, from about 0.5 to 10 mg/kg, from about 1 to 8 mg/kg, from about 1.5 to 10 mg/kg, from about 2 to 12 mg/kg, from about 2 to 10 mg/kg, from about 3 to 10 mg/kg, from about 4 to 10 mg/kg, from about 4 to 12 mg/kg, or from about 5 to 12 mg/kg, or about 0.001 mg/kg, 0.0025 mg/kg, 0.005 mg/kg, 0.01 mg/kg, 0.025 mg/kg, 0.05 mg/kg, 0.1 mg/kg, 0.25, 0.5 mg/kg, 1.0 mg/kg, 2.0 mg/kg, 4.0 mg/kg, 6 mg/kg, 8.0 mg/kg, 10 mg/kg, or 12 mg/kg. In various embodiments, TIMP-PPE is administered at a dose of about 0.1 mg, 0.25 mg, 0.5 mg, 1 mg, 2 mg, 2.5 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg. In various embodiments, the anti-IgE antibody administered in combination with TIMP-PPE is administered at a dose of about 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, or 500 mg. In various embodiments, the dose level of the anti-IgE antibody is determined based on serum IgE levels. In various embodiments, the serum IgE levels are between 30-100 IU/mL, 100-200 IU/mL, 200-300 IU/mL, 300-400 IU/mL, 400-500 IU/mL, 500-600 IU/mL, 600-700 IU/mL, 700-800 IU/mL, 800-900 IU/mL, 900-1000 IU/mL, 1000-1100 IU/mL, 1100-1200 IU/mL, 1200-1300 IU/mL, 1300-1400 IU/mL, or between 1400-1500 IU/mL. In various embodiments, the dose level of the anti-IgE antibody is determined based on the weight of the subject. In various embodiments, the weight of the subject is between 30-40 kg, 40-50 kg, 50-60 kg, 60-70 kg, 70-80 kg, 80-90 kg, 90-125 kg, or between 125-150 kg.

In various embodiments, the anti-IgE antibody is administered in a single dose or in multiple doses. In various embodiments, the anti-IgE antibody is administered once weekly, once every two weeks, once every three weeks, or once every four weeks. In various embodiments, the anti-IgE antibody is administered prior to, concomitantly, or after the administration of TIMP-PPE. In various embodiments, the anti-IgE antibody is administered for one week, two weeks, three weeks, four weeks, 1 month, 2 months, 3 months, 4 months, 5 months, 6 months, 7 months, 8 months, 9 months, 10 months, 11 months, or 12 months prior to the administration of TIMP-PPE. In various embodiments, the anti-IgE antibody is administered for one week, two weeks, three weeks, or four weeks after the administration of TIMP-PPE.

The present disclosure provides methods for treating Peanut Allergy comprising administering to a subject TIMP-PPE in combination with anti-IgE antibody, wherein the subject is administered anti-IgE antibody once weekly for two weeks or once weekly for four weeks prior to the administration of TIMP-PPE, wherein TIMP-PPE is administered in two doses one-week apart at a dose level of between 0.001 mg/kg and 12 mg/kg, and wherein the anti-IgE antibody is administered at a dose level of between about 50 mg and 500 mg.

The present disclosure provides methods for treating peanut allergy comprising administering to a subject TIMP-PPE in combination with anti-IgE antibody, wherein the subject is administered anti-IgE antibody once weekly for two weeks or once weekly for four weeks prior to the administration of TIMP-PPE, wherein TIMP-PPE is administered in two doses one-week apart at a dose level of between 0.1 mg and 800 mg, and wherein the anti-IgE antibody is administered at a dose level of between about 50 mg and 500 mg.

The present disclosure describes a method of treating Peanut Allergy in a subject comprising administering to the subject TIMP-PPE in combination with an anti-IL-4Rα antibody, wherein TIMP-PPE is administered at a dose of about 0.001 mg/kg to 12 mg/kg, and wherein the anti-IL-4Rα antibody is administered at a dose of about 10 mg to about 500 mg. In various embodiments, the anti-IL-4Rα antibody is Dupilumab (DUPIXENT®). In various embodiments, TIMP-PPE is administered at a dose from about 0.001 to 10 mg/kg, from about 0.005 to 12 mg/kg, from about 0.01 to 12 mg/kg, from about 0.05 to 12 mg/kg, from about 0.1 to 12 mg/kg, 0.5 to 10 mg/kg, from about 1 to 8 mg/kg, from about 1.5 to 10 mg/kg, from about 2 to 12 mg/kg, from about 2 to 10 mg/kg, from about 3 to 10 mg/kg, from about 4 to 10 mg/kg, from about 4 to 12 mg/kg, or from about 5 to 12 mg/kg, or about 0.001 mg/kg, 0.0025 mg/kg, 0.005 mg/kg, 0.01 mg/kg, 0.025 mg/kg, 0.05 mg/kg, 0.1 mg/kg, 0.25, 0.5 mg/kg, 1.0 mg/kg, 2.0 mg/kg, 4.0 mg/kg, 6 mg/kg, 8.0 mg/kg, 10 mg/kg, or 12 mg/kg. In various embodiments, TIMP-PPE is administered at a dose of about 0.1 mg, 0.25 mg, 0.5 mg, 1 mg, 2 mg, 2.5 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg. In various embodiments, the anti-IL-4Rα antibody is administered at a dose of about 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, 500 mg, 525 mg, 550 mg, 575 mg, or 600 mg. In various embodiments, the dose level of the anti-IL-4Rα antibody is determined based on serum IgE levels. In various embodiments, the serum IgE levels are between 30-100 IU/mL, 100-200 IU/mL, 200-300 IU/mL, 300-400 IU/mL, 400-500 IU/mL, 500-600 IU/mL, 600-700 IU/mL, 700-800 IU/mL, 800-900 IU/mL, 900-1000 IU/mL, 1000-1100 IU/mL, 1100-1200 IU/mL, 1200-1300 IU/mL, 1300-1400 IU/mL, or between 1400-1500 IU/mL. In various embodiments, the anti-IL-4Rα antibody dose level is determined based on the weight of the subject. In various embodiments, the weight of the subject is between 30-40 kg, 40-50 kg, 50-60 kg, 60-70 kg, 70-80 kg, 80-90 kg, 90-125 kg, or between 125-150 kg. In various embodiments, the anti-IL-4Rα antibody is administered in a single dose or in multiple doses. In various embodiments, the anti-IL-4Rα antibody is administered once weekly, once every two weeks, once every three weeks, or once every four weeks. In various embodiments, the anti-IL-4Rα antibody is administered prior to, concomitantly, or after the administration of TIMP-PPE. In various embodiments, the anti-IL-4Rα antibody is administered for one week, two weeks, three weeks, or four weeks prior to the administration of TIMP-PPE. In various embodiments, the anti-IL-4Rα antibody is administered for one week, two weeks, three weeks, or four weeks after the administration of TIMP-PPE. In various embodiments, the anti-IL-4Rα antibody is administered at an initial dose level of between 400 mg and 600 mg for two doses followed by a maintenance dose level of between 200 mg and 300 mg for subsequent doses.

In various embodiments, the antihistamine administered in combination with TIMP-PPE is administered at a dose of about 0.05 mg to 2000 mg. In various embodiments, the antihistamine is administered at a dose of about 0.05 mg, 0.1 mg, 0.5 mg, 1 mg, 2 mg, 4 mg, 5 mg, 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, 500 mg, 1000 mg, 1500 mg, or 2000 mg. In various embodiments, the antihistamine is administered in a single dose or in multiple doses. In various embodiments, the antihistamine is administered once daily, once weekly, once every two weeks, once every three weeks, or once every four weeks. In various embodiments, the antihistamine is administered two times, three times, four times, five times, or six times daily. In various embodiments, the antihistamine is administered prior to, concomitantly, or after the administration of TIMP-PPE. In various embodiments, the antihistamine is administered for one week, two weeks, three weeks, four weeks, 1 month, 2 months, 3 months, 4 months, 5 months, 6 months, 7 months, 8 months, 9 months, 10 months, 11 months, or 12 months prior to the administration of TIMP-PPE. In various embodiments, the antihistamine is administered for one week, two weeks, three weeks, four weeks, 1 month, 2 months, 3 months, 4 months, 5 months, 6 months, 7 months, 8 months, 9 months, 10 months, 11 months, or 12 months after the administration of TIMP-PPE. In various embodiments, the antihistamine is administered 5, 10, 15, 30, 45, or 60 minutes prior to the administration of TIMP-PPE. In various embodiments, the antihistamine is administered 5, 10, 15, 30, 45, or 60 minutes after the administration of TIMP-PPE. In various embodiments, the antihistamine is administered 1, 2, 3, 4, 5, 6, 7, 8, 9, 10, 11, 12, 13, 14, 15, 16, 17, 18, 19, 20, 21, 22, 23, 24, 36, 48, 72, or 96 hours prior to the administration of TIMP-PPE. In various embodiments, the antihistamine is administered 1, 2, 3, 4, 5, 6, 7, 8, 9, 10, 11, 12, 13, 14, 15, 16, 17, 18, 19, 20, 21, 22, 23, 24, 36, 48, 72, or 96 hours after the administration of TIMP-PPE. In various embodiments, the antihistamine is administered 1, 2, 3, 4, 5, 6, or 7 days prior to the administration of TIMP-PPE. In various embodiments, the antihistamine is administered 1, 2, 3, 4, 5, 6, or 7 days after the administration of TIMP-PPE.

In various embodiments, the steroid administered in combination with TIMP-PPE is administered at a dose of about 0.05 mg to 2000 mg. In various embodiments, the steroid is administered at a dose of about 0.05 mg, 0.1 mg, 0.5 mg, 1 mg, 2 mg, 4 mg, 5 mg, 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, 500 mg, 1000 mg, 1500 mg, or 2000 mg. In various embodiments, the steroid is administered in a single dose or in multiple doses. In various embodiments, the steroid is administered once daily, once weekly, once every two weeks, once every three weeks, or once every four weeks. In various embodiments, the steroid is administered two times, three times, four times, five times, or six times daily. In various embodiments, the steroid is administered prior to, concomitantly, or after the administration of TIMP-PPE. In various embodiments, the steroid is administered for one week, two weeks, three weeks, four weeks, 1 month, 2 months, 3 months, 4 months, 5 months, 6 months, 7 months, 8 months, 9 months, 10 months, 11 months, or 12 months prior to the administration of TIMP-PPE. In various embodiments, the steroid is administered for one week, two weeks, three weeks, four weeks, 1 month, 2 months, 3 months, 4 months, 5 months, 6 months, 7 months, 8 months, 9 months, 10 months, 11 months, or 12 months after the administration of TIMP-PPE. In various embodiments, the steroid is administered 5, 10, 15, 30, 45, or 60 minutes prior to the administration of TIMP-PPE. In various embodiments, the steroid is administered 1, 2, 3, 4, 5, 6, 7, 8, 9, 10, 11, 12, 13, 14, 15, 16, 17, 18, 19, 20, 21, 22, 23, 24, 36, 48, 72, or 96 hours prior to the administration of TIMP-PPE. In various embodiments, the steroid is administered 1, 2, 3, 4, 5, 6, or 7 days prior to the administration of TIMP-PPE.

In various embodiments, the corticosteroid administered in combination with TIMP-PPE is administered at a dose of about 0.05 mg to 2000 mg. In various embodiments, the corticosteroid is administered at a dose of about 0.05 mg, 0.1 mg, 0.5 mg, 1 mg, 2 mg, 4 mg, 5 mg, 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, 500 mg, 1000 mg, 1500 mg, or 2000 mg. In various embodiments, the corticosteroid is administered in a single dose or in multiple doses. In various embodiments, the corticosteroid is administered once daily, once weekly, once every two weeks, once every three weeks, or once every four weeks. In various embodiments, the corticosteroid is administered two times, three times, four times, five times, or six times daily. In various embodiments, the corticosteroid is administered prior to, concomitantly, or after the administration of TIMP-PPE. In various embodiments, the corticosteroid is administered for one week, two weeks, three weeks, four weeks, 1 month, 2 months, 3 months, 4 months, 5 months, 6 months, 7 months, 8 months, 9 months, 10 months, 11 months, or 12 months prior to the administration of TIMP-PPE. In various embodiments, the corticosteroid is administered for one week, two weeks, three weeks, four weeks, 1 month, 2 months, 3 months, 4 months, 5 months, 6 months, 7 months, 8 months, 9 months, 10 months, 11 months, or 12 months after the administration of TIMP-PPE. In various embodiments, the corticosteroid is administered 5, 10, 15, 30, 45, or 60 minutes prior to the administration of TIMP-PPE. In various embodiments, the corticosteroid is administered 5, 10, 15, 30, 45, or 60 minutes after the administration of TIMP-PPE. In various embodiments, the corticosteroid is administered 1, 2, 3, 4, 5, 6, 7, 8, 9, 10, 11, 12, 13, 14, 15, 16, 17, 18, 19, 20, 21, 22, 23, 24, 36, 48, 72, or 96 hours prior to the administration of TIMP-PPE. In various embodiments, the corticosteroid is administered 1, 2, 3, 4, 5, 6, 7, 8, 9, 10, 11, 12, 13, 14, 15, 16, 17, 18, 19, 20, 21, 22, 23, 24, 36, 48, 72, or 96 hours after the administration of TIMP-PPE. In various embodiments, the corticosteroid is administered 1, 2, 3, 4, 5, 6, or 7 days prior to the administration of TIMP-PPE. In various embodiments, the corticosteroid is administered 1, 2, 3, 4, 5, 6, or 7 days after the administration of TIMP-PPE.

In various embodiments, the NSAID administered in combination with TIMP-PPE is administered at a dose of about 0.05 mg to 2000 mg. In various embodiments, the NSAID is administered at a dose of about 0.05 mg, 0.1 mg, 0.5 mg, 1 mg, 2 mg, 4 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 1000 mg, 1500 mg, or 2000 mg. In various embodiments, the NSAID is administered in a single dose or in multiple doses. In various embodiments, the NSAID is administered once daily, once weekly, once every two weeks, once every three weeks, or once every four weeks. In various embodiments, the NSAID is administered two times, three times, four times, five times, or six times daily. In various embodiments, the NSAID is administered prior to, concomitantly, or after the administration of TIMP-PPE. In various embodiments, the NSAID is administered for one week, two weeks, three weeks, four weeks, 1 month, 2 months, 3 months, 4 months, 5 months, 6 months, 7 months, 8 months, 9 months, 10 months, 11 months, or 12 months prior to the administration of TIMP-PPE. In various embodiments, the NSAID is administered for one week, two weeks, three weeks, four weeks, 1 month, 2 months, 3 months, 4 months, 5 months, 6 months, 7 months, 8 months, 9 months, 10 months, 11 months, or 12 months after the administration of TIMP-PPE. In various embodiments, the NSAID is administered 5, 10, 15, 30, 45, or 60 minutes prior to the administration of TIMP-PPE. In various embodiments, the NSAID is administered 5, 10, 15, 30, 45, or 60 minutes after the administration of TIMP-PPE. In various embodiments, the NSAID is administered 1, 2, 3, 4, 5, 6, 7, 8, 9, 10, 11, 12, 13, 14, 15, 16, 17, 18, 19, 20, 21, 22, 23, 24, 36, 48, 72, or 96 hours prior to the administration of TIMP-PPE. In various embodiments, the NSAID is administered 1, 2, 3, 4, 5, 6, 7, 8, 9, 10, 11, 12, 13, 14, 15, 16, 17, 18, 19, 20, 21, 22, 23, 24, 36, 48, 72, or 96 hours after the administration of TIMP-PPE. In various embodiments, the NSAID is administered 1, 2, 3, 4, 5, 6, or 7 days prior to the administration of TIMP-PPE. In various embodiments, the NSAID is administered 1, 2, 3, 4, 5, 6, or 7 days after the administration of TIMP-PPE.

In various embodiments, the leukotriene modifier administered in combination with TIMP-PPE is administered at a dose of about 0.05 mg to 2000 mg. In various embodiments, the leukotriene modifier is administered at a dose of about 0.05 mg, 0.1 mg, 0.5 mg, 1 mg, 2 mg, 4 mg, 5 mg, 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, 500 mg, 1000 mg, 1500 mg, or 2000 mg. In various embodiments, the leukotriene modifier is administered in a single dose or in multiple doses. In various embodiments, the leukotriene modifier is administered once daily, once weekly, once every two weeks, once every three weeks, or once every four weeks. In various embodiments, the leukotriene modifier is administered two times, three times, four times, five times, or six times daily. In various embodiments, the leukotriene modifier is administered prior to, concomitantly, or after the administration of TIMP-PPE. In various embodiments, the leukotriene modifier is administered for one week, two weeks, three weeks, four weeks, 1 month, 2 months, 3 months, 4 months, 5 months, 6 months, 7 months, 8 months, 9 months, 10 months, 11 months, or 12 months prior to the administration of TIMP-PPE. In various embodiments, the leukotriene modifier is administered for one week, two weeks, three weeks, four weeks, 1 month, 2 months, 3 months, 4 months, 5 months, 6 months, 7 months, 8 months, 9 months, 10 months, 11 months, or 12 months after the administration of TIMP-PPE. In various embodiments, the leukotriene modifier is administered 5, 10, 15, 30, 45, or 60 minutes prior to the administration of TIMP-PPE. In various embodiments, the leukotriene modifier is administered 5, 10, 15, 30, 45, or 60 minutes after the administration of TIMP-PPE. In various embodiments, the leukotriene modifier is administered 1, 2, 3, 4, 5, 6, 7, 8, 9, 10, 11, 12, 13, 14, 15, 16, 17, 18, 19, 20, 21, 22, 23, 24, 36, 48, 72, or 96 hours prior to the administration of TIMP-PPE. In various embodiments, the leukotriene modifier is administered 1, 2, 3, 4, 5, 6, 7, 8, 9, 10, 11, 12, 13, 14, 15, 16, 17, 18, 19, 20, 21, 22, 23, 24, 36, 48, 72, or96 hours prior after the administration of TIMP-PPE. In various embodiments, the leukotriene modifier is administered 1, 2, 3, 4, 5, 6, or 7 days prior to the administration of TIMP-PPE. In various embodiments, the leukotriene modifier is administered 1, 2, 3, 4, 5, 6, or 7 days after the administration of TIMP-PPE.

It is contemplated that when TIMP-PPE is administered with an additional therapeutic as described herein, the TIMP-PPE is administered in a single dose or in multiple doses. In various embodiments, TIMP-PPE is administered in two doses one-week apart.

It is contemplated that when TIMP-PPE is administered in combination with a therapeutic useful to treat peanut allergy, as described herein, the TIMP-PPE is administered in a single dose or in multiple doses. In various embodiments, TIMP-PPE is administered in two doses one-week apart. In various embodiments, TIMP-PPE is administered in two doses one-week apart followed by a booster dose of TIMP-PPE administered as a single dose once every three months.

In various embodiments, administering TIMP-PPE to a subject in need thereof, alone or in combination with one or more additional therapeutics, relieves one or more symptoms of Peanut Allergy. In various embodiments, the symptoms of Peanut Allergy are selected from the group consisting of skin reactions, hives, skin redness, skin swelling, itching, tightening of the throat, difficulty breathing, shortness of breath, and anaphylaxis.

In various embodiments, administering TIMP-PPE to a subject in need thereof, alone or in combination with one or more therapeutics useful to treat peanut allergy, relieves one or more symptoms of Peanut Allergy. In various embodiments, the symptoms of Peanut Allergy are selected from the group consisting of skin reactions, hives, skin redness, skin swelling, itching, tightening of the throat, difficulty breathing, shortness of breath, digestive problems such as diarrhea, stomach cramps, nausea, or vomiting, drop in blood pressure, and anaphylaxis.

In various embodiments, administering TIMP-PPE to a subject in need thereof, alone or in combination with one or more additional therapeutics, reduces the duration and severity of an allergic immune response to peanut proteins. In various embodiments, administering TIMP-PPE to a subject in need thereof, alone or in combination with one or more additional therapeutics, reduces the duration and severity of an allergic immune response following exposure to peanut proteins. In various embodiments, the allergic immune response is a Th2 T cell response, B-cell activation, basophil activation, eosinophil activation, mast cell activation, and/or IgE induction.

In various embodiments, the efficacy of TIMP-PPE at relieving one or more symptoms of Peanut Allergy and/or reducing the duration and severity of an allergic immune response to peanut proteins is determined from the assay of one or more biological samples from the subject. In various embodiments, the biological samples are selected from the group consisting whole-blood, peripheral blood, peripheral blood mononuclear cells (PBMCs), serum, plasma, urine, cerebrospinal fluid (CSF), stool, a tissue biopsy, and/or a bone-marrow biopsy. In various embodiments, the efficacy of TIMP-PPE at relieving one or more symptoms of peanut allergy and/or reducing the duration and severity of an allergic immune response to peanut proteins is determined by a double-blind placebo-controlled food challenge (DBPCFC). In various embodiments, the efficacy of TIMP-PPE at relieving one or more symptoms of Peanut Allergy and/or reducing the duration and severity of an allergic immune response to peanut proteins is determined by a skin prick test (SPT).

In various embodiments, administering TIMP-PPE alone or in combination with an additional therapeutic to the subject reduces the proportion of Th2a+ T cells present in the total T cell population in peripheral blood.

In various embodiments, administering TIMP-PPE alone or in combination with a therapeutic to the subject decreases the proportion of Th2a+ T cells present in the total T cell population in peripheral blood.

In various embodiments, administering TIMP-PPE alone or in combination with an additional therapeutic to the subject reduces the ratio of activated peanut protein specific T cells to unactivated peanut protein specific T cells in peripheral blood.

In various embodiments, administering TIMP-PPE alone or in combination with a therapeutic to the subject decreases the proportion of activated peanut protein specific T cells in peripheral blood.

In various embodiments, administering TIMP-PPE alone or in combination with an additional therapeutic to the subject increases the levels of peanut protein specific Treg cells in blood. In various embodiments, administering TIMP-PPE alone or in combination with a therapeutic to the subject increases the levels of peanut protein specific Treg cells in blood.

In various embodiments, administering TIMP-PPE alone or in combination with an additional therapeutic to the subject reduces basophil activation. In various embodiments, administering TIMP-PPE alone or in combination with a therapeutic to the subject decreases basophil activation.

In various embodiments, administering TIMP-PPE alone or in combination with an additional therapeutic to the subject reduces peanut protein specific IgE levels in blood. In various embodiments, administering TIMP-PPE alone or in combination with a therapeutic to the subject reduces peanut protein specific IgE levels in blood.

In various embodiments, administering TIMP-PPE alone or in combination with an additional therapeutic to the subject reduces the ratio of peanut protein specific IgE to IgG levels in blood. In various embodiments, administering TIMP-PPE alone or in combination with a therapeutic to the subject reduces the ratio of peanut protein specific IgE to IgG levels in blood.

In various embodiments, administering TIMP-PPE alone or in combination with an additional therapeutic to the subject reduces the levels of Th2 cytokine levels in blood In various embodiments, administering TIMP-PPE alone or in combination with a therapeutic to the subject reduces the levels of Th2 cytokine levels in blood. In various embodiments, the Th2 cytokines are selected from the group consisting of IL-4, IL-5, IL-9, and IL-13.

In various embodiments, administering TIMP-PPE alone or in combination with an additional therapeutic to the subject increases tolerance to peanut proteins. In various embodiments, administering TIMP-PPE alone or in combination with a therapeutic to the subject increases tolerance to peanut proteins.

Also contemplated is a composition comprising TIMP-PPE as described herein for use in treating peanut allergy. In various embodiments, the disclosure provides for use of a composition comprising TIMP-PPE as described herein in the preparation of a medicament for treating peanut allergy.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

The headings herein are for the convenience of the reader and not intended to be limiting. Additional aspects, embodiments, and variations of the invention will be apparent from the Detailed Description and/or Drawing and/or claims.

### BRIEF DESCRIPTION OF THE DRAWINGS

FIG. 1A. Schedule of events for subjects participating in Part A of the human study of CNP-201 particles. FIG. 1B. Schedule of events for subjects participating in Part B of the human study of CNP-201 particles. FIG. 1C. Schedule of events for subjects participating in the low starting dose study of CNP-201 particles.

FIG. 2. Summary of results for patients receiving low dose CNP-201 particles in peanut allergy: Patients received approximately one dose of 1 mg or 25 mg of CNP-201 intravenously. Patients receiving low dose CNP-201 had increased BAT thresholds, decrease in ratio of peanut specific IgE to IgG, induction of antigen specific Tregs and/or decrease in pathogenic peanut specific T cell subsets (Th2a, TFH, B cell plasma blasts).

FIG. 3. Basophil sensitivity test with peanut crude extract: Peanut allergic patient receiving one 25 mg intravenous dose of CNP-201 showed an increase in the EC50 (concentration of peanut allergen) needed to increase expression of activation markers on the surface of basophils following stimulation with peanut allergen.

FIG. 4. Shift of IgE to IgG phenotype with up to 25 mg CNP-201 dose: Peanut allergic patients receiving up to 25 mg intravenous dose of CNP-201 showed an increase in peanut specific IgG and a decrease in peanut specific IgE/IgG ratio ar day 15 and day 38 after dosing compared to placebo.

FIG. 5. Reduced peanut allergy associated immune cell subsets. Peanut allergic patient receiving one 25 mg intravenous dose of CNP-201 showed a decrease in allergy-promoting subsets T helper 2A (Th2A), T follicular helper cell (TFH), terminally differentiated effector memory cells (TEMRA) and B cell plasma blasts.

FIG. 6. Lower pathogenic activated peanut specific CD4+ T cells observed after a single 25 mg dose of CNP-201. There was a decrease in the peanut specific activated T cell subsets CD4+CD25+, CD4+CD69+ and CD4+PD-1+ compared to placebo in the low dose CNP-201 treated patient.

FIG. 7. Induction of antigen specific regulatory T cells (Tregs): Peanut allergic patient receiving one 25 mg intravenous dose of CNP-201 showed an increase in peanut specific Tregs compared to placebo.

FIG. 8. Schedule of events for subjects participating in the 1 mg starting dose study of CNP-201 particles.

### DETAILED DESCRIPTION

The present disclosure provides methodology for monitoring the induction of and maintenance of immunologic tolerance in a subject after receiving immunotherapy.

### Definitions

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

“Particle” as used herein refers to any non-tissue derived composition of matter, it may be a sphere or sphere-like entity, bead, or liposome. The term “particle”, the term “immune modifying particle”, the term “carrier particle”, and the term “bead” may be used interchangeably depending on the context. Additionally, the term “particle” may be used to encompass beads and spheres.

“Negatively charged particle” as used herein refers to particles which have been modified to possess a net surface charge that is less than zero.

“Carboxylated particles” or “carboxylated beads” or “carboxylated spheres” includes any particle that has been modified to contain a carboxyl group on its surface. In some embodiments the addition of the carboxyl group enhances phagocyte/monocyte uptake of the particles from circulation, for instance through the interaction with scavenger receptors such as MARCO. Carboxylation of the particles can be achieved using any compound which adds carboxyl groups.

“TIMP-PPE” as used herein refers to a negatively charged tolerizing immune modifying particle (TIMP) comprising a peanut extract or one or more peanut proteins or antigenic fragments thereof, including Ara h1, Ara h2, Ara h3, Ara h5, Ara h6, Ara h7, Ara h8, Ara h9, Ara h10, Ara h11, Ara h12, Ara h13, Ara h14, Ara h15, Ara h16, Ara h17, and Ara h18.

As used herein, the term “Th cell” or “helper T cell” refers to CD4⁺ cells. CD4⁺ T cells assist other white blood cells with immunologic processes, including maturation of B cells into plasma cells and memory B cells, and activation of cytotoxic T cells and macrophages. T cells become activated when they are presented with peptide antigens by MHC class II molecules, which are expressed on the surface of antigen-presenting cells (APCs).

As used herein, the term “Th1 cell” refers to a subset of Th cells which produce proinflammatory mediators. Th1 cells secrete cytokines to facilitate immune response and play a role in host defense against pathogens in part by mediating the recruitment of neutrophils and macrophages to infected tissues. Th1 cells secrete cytokines including IFN-gamma, IL-2, IL-10, and TNF alpha/beta to coordinate defense against intracellular pathogens such as viruses and some bacteria.

As used herein, the term “Th2 cell” refers to a subset of Th cells that mediate the activation and maintenance of the antibody-mediated immune response against extracellular parasites, bacteria, allergens, and toxins. Th2 cells mediate these functions by producing various cytokines such as IL-4, IL-5, IL-6, IL-9, IL-13, and IL-17E (IL-25) that are responsible for antibody production, eosinophil activation, and inhibition of several macrophage functions, thus providing phagocyte-independent protective responses.

“Polypeptide” and “protein” refer to a polymer composed of amino acid residues, related naturally occurring structural variants, and synthetic non-naturally occurring analogs thereof, linked via peptide bonds or peptide bond isosteres. Synthetic polypeptides can be synthesized, for example, using an automated polypeptide synthesizer. The terms “polypeptide” and “protein” are not limited to a minimum length of the product. The term “protein” typically refers to large polypeptides. The term “peptide” typically refers to short polypeptides. Thus, peptides, oligopeptides, dimers, multimers, and the like, are included within the definition. Both full-length proteins and fragments thereof are encompassed by the definition. The terms “polypeptide” and “protein” also include post-expression modifications of the polypeptide or protein, for example, glycosylation, acetylation, phosphorylation and the like. Furthermore, for purposes of the present disclosure, a “polypeptide” can include “modifications,” such as deletions, additions, substitutions (which may be conservative in nature or may include substitutions with any of the 20 amino acids that are commonly present in human proteins, or any other naturally or non-naturally-occurring or atypical amino acids), and chemical modifications (e.g., addition of or substitution with peptidomimetics), to the native sequence. These modifications may be deliberate, as through site-directed mutagenesis, or through chemical modification of amino acids to remove or attach chemical moieties, or may be accidental, such as through mutations arising via hosts cells that produce the proteins or through errors due to PCR amplification prior to host cell transfection.

“Antigenic moiety” or “antigen” as used herein refers to any moiety, for example a peptide, that is recognized by the host's immune system. Examples of antigenic moieties include, but are not limited to, autoantigens, allergens, enzymes, and/or bacterial or viral proteins, peptides, drugs or components.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration) or via inhalation.

By “pharmaceutically acceptable” or “pharmacologically acceptable” is meant a material that is not biologically or otherwise undesirable, i.e., the material may be administered to an individual without causing any undesirable biological effects or without interacting in a deleterious manner with any of the components of the composition in which it is contained or with any components present on or in the body of the individual.

As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

The term “epitope” refers to that portion of any molecule capable of being recognized by and bound by a selective binding agent at one or more of the antigen binding regions. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous. Moreover, epitopes may be mimetic (mimotopes) in that they comprise a three-dimensional structure that is identical to the epitope used to generate the antibody, yet comprise none or only some of the amino acid residues found in the target that were used to stimulate the antibody immune response. As used herein, a mimotope is not considered a different antigen from the epitope bound by the selective binding agent; the selective binding agent recognizes the same three-dimensional structure of the epitope and mimotope.

The term “therapeutically effective amount” is used herein to indicate the amount of antigen-specific composition of the disclosure that is effective to ameliorate or lessen symptoms or signs of disease to be treated.

The terms “treat”, “treated”, “treating” and “treatment”, as used with respect to methods herein refer to eliminating, reducing, suppressing or ameliorating, either temporarily or permanently, either partially or completely, a clinical symptom, manifestation or progression of an event, disease or condition. Such treating need not be absolute to be useful.

The term “symptom’ as used herein refers to any physical or observable manifestation of a disorder, whether it is generally characteristic of that disorder or not. The term “symptoms” can mean all such manifestations or any subset thereof.

“Booster dose” as used herein refers to re-dosing of TIMPs after the original or initial administration. Administration or re-administration of the booster dose enhances the tolerogenic immune response induced by the original administration. A booster dose may be more than, the same as or less than the initial dose of TIMP particles administered to the subject. Subjects treated with tolerizing therapies are monitored to confirm the maintenance of immunological tolerance. The decision to administer a booster dose is based on observation of changes, weakening, or loss of immune tolerance. Methods for monitoring immune tolerance status of a subject treated with tolerizing therapies have been previously described (See International Patent Publication WO 2022/221622, incorporated herein by reference). Administration of the booster dose of TIMP-PPE is done with the same, different or none of the combination therapies described herein that are capable of administration with the original TIMP-PPE doses.

### Particles

The size and charge of the particles are important for tolerance induction. While the particles will differ in size and charge based on the antigen encapsulated within them, in general, particles described herein are effective at inducing tolerance when they are between about 100 nanometers and about 1500 nanometers and have a charge of between 0 to about −100 mV. In various embodiments, the particles are 400-800 nanometers in diameter and have a charge of between about −25 mV and −70 mV. In various embodiments, the particles are 400-800 nanometers in diameter and have a charge of between about −30 mV and −60 mV. In various embodiments, the particles are 400-800 nanometers in diameter and have a charge of between about −30 mV and −80 mV. The average particle size and charge of the particles can be slightly altered in the lyophilization process, therefore, both post-synthesis averages and post-lyophilization averages are described. As used herein, the term “post-synthesis size” and “post synthesis charge” refer to the size and charge of the particle prior to lyophilization. The term “post lyophilization size” and “post lyophilization charge” refer to the size and charge of the particle after lyophilization.

In some embodiments, the particle is non-metallic. In these embodiments the particle may be formed from a polymer. In a preferred embodiment, the particle is biodegradable in an individual. In this embodiment, the particles can be provided in an individual across multiple doses without there being an accumulation of particles in the individual. Examples of suitable particles include polystyrene particles, PLGA particles, PLURONICS stabilized polypropylene sulfide particles, and diamond particles.

Preferably the particle surface is composed of a material that minimizes non-specific or unwanted biological interactions. Interactions between the particle surface and the interstitium may be a factor that plays a role in lymphatic uptake. The particle surface may be coated with a material to prevent or decrease non-specific interactions. Steric stabilization by coating particles with hydrophilic layers such as poly(ethylene glycol) (PEG) and its copolymers such as PLURONICS® (including copolymers of poly(ethylene glycol)-bl-poly(propylene glycol)-bl-poly(ethylene glycol)) may reduce the non-specific interactions with proteins of the interstitium as demonstrated by improved lymphatic uptake following subcutaneous injections. All of these facts suggest relevance of the physical properties of the particles in terms of lymphatic uptake. Biodegradable polymers may be used to make all or some of the polymers and/or particles and/or layers. Biodegradable polymers may undergo degradation, for example, by a result of functional groups reacting with the water in the solution. The term “degradation” as used herein refers to becoming soluble, either by reduction of molecular weight or by conversion of hydrophobic groups to hydrophilic groups. Polymers with ester groups are generally subject to spontaneous hydrolysis, e.g., polylactides and polyglycolides.

Particles disclosed herein may also contain additional components. For example, carriers may have imaging agents incorporated or conjugated to the carrier. An example of a carrier nanosphere having an imaging agent that is currently commercially available is the Kodak X-sight nanospheres. Inorganic quantum-confined luminescent nanocrystals, known as quantum dots (QDs), have emerged as ideal donors in FRET applications: their high quantum yield and tunable size-dependent Stokes Shifts permit different sizes to emit from blue to infrared when excited at a single ultraviolet wavelength. (Bruchez, et al., Science, 1998, 281, 2013; Niemeyer, C. M Angew. Chem. Int. Ed. 2003, 42, 5796; Waggoner, A. Methods Enzymol. 1995, 246, 362; Brus, L. E. J. Chem. Phys. 1993, 79, 5566). Quantum dots, such as hybrid organic/inorganic quantum dots based on a class of polymers known as dendrimers, may be used in biological labeling, imaging, and optical biosensing systems. (Lemon, et al., J. Am. Chem. Soc. 2000, 122, 12886). Unlike the traditional synthesis of inorganic quantum dots, the synthesis of these hybrid quantum dot nanoparticles does not require high temperatures or highly toxic, unstable reagents. (Etienne, et al., Appl. Phys. Lett. 87, 181913, 2005).

Particles can be formed from a wide range of materials. The particle is preferably composed of a material suitable for biological use. For example, particles may be composed of glass, silica, polyesters of hydroxy carboxylic acids, polyanhydrides of dicarboxylic acids, or copolymers of hydroxy carboxylic acids and dicarboxylic acids. More generally, the carrier particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, carrier particles can be quantum dots, or composed of quantum dots, such as quantum dot polystyrene particles (Joumaa et al. (2006) Langmuir 22: 1810-6). Carrier particles including mixtures of ester and anhydride bonds (e.g., copolymers of glycolic and sebacic acid) may also be employed. For example, carrier particles may comprise materials including polyglycolic acid polymers (PGA), polylactic acid polymers (PLA), polysebacic acid polymers (PSA), poly(lactic-co-glycolic) acid copolymers (PLGA or PLG; the terms are interchangeable), poly(lactic-co-sebacic) acid copolymers (PLSA), poly(glycolic-co-sebacic) acid copolymers (PGSA), polypropylene sulfide polymers, poly(caprolactone), chitosan, etc. Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include diamond, PLA, PGA, polypropylene sulfide, and PLGA polymers. Biocompatible but non-biodegradable materials may also be used in the carrier particles of the invention. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON© (DuPont, Wilmington, Del.), and nylons may be employed.

In certain embodiments, the particle is a co-polymer having a molar ratio from about 80:20 to about 100:0. Suitable co-polymer ratio of present immune modified particles may be 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0. In certain embodiments, the particle is a PLURONICS stabilized polypropylene sulfide particle, a polyglycolic acid particle (PGA), a polylactic acid particle (PLA), or a poly(lactic-co-glycolic acid) particle. In certain embodiments, the particle has a copolymer ratio of polylactic acid/polyglycolic acid 80:20: polylactic acid/polyglycolic acid 90:10: or polylactic acid: polyglycolic acid/50:50. In various embodiments, the particle is a poly(lactic-co-glycolic acid) particle and has a copolymer ratio of about 50:50 polylactic acid:polyglycolic acid.

It is contemplated that the particle may further comprise a surfactant. The surfactant can be anionic, cationic, or nonionic. Surfactants in the poloxamer and poloaxamines family are commonly used in particle synthesis. Surfactants that may be used, include, but are not limited to PEG, Tween-80, gelatin, dextran, pluronic L-63, PVA, PAA, methylcellulose, lecithin, DMAB and PEMA. Additionally, biodegradable and biocompatible surfactants including, but not limited to, vitamin E TPGS (D-α-tocopheryl polyethylene glycol 1000 succinate), poly amino acids (e.g polymers of lysine, arginine, aspartic acid, glutamic acid, serine, threonine, tyrosine and cysteine, or their enantiomers), and sulfate polymers. In certain embodiments, two surfactants are used. For example, if the particle is produced by a double emulsion method, the two surfactants can include a hydrophobic surfactant for the first emulsion, and a hydrophobic surfactant for the second emulsion.

In certain embodiments, the polypeptide antigens are encapsulated in the particles by a single-emulsion process. In a further embodiment, the polypeptide antigens are more hydrophobic. Sometimes, the double emulsion process leads to the formation of large particles which may result in the leakage of the hydrophilic active component and low entrapment efficiencies. The coalescence and Ostwald ripening are two mechanisms that may destabilize the double-emulsion droplet, and the diffusion through the organic phase of the hydrophilic active component is the main mechanism responsible of low levels of entrapped active component. In some embodiments, it may be beneficial to reduce the nanoparticle size. One strategy to accomplish this is to apply a second strong shear rate. The leakage effect can be reduced by using a high polymer concentration and a high polymer molecular mass, accompanied by an increase in the viscosity of the inner water phase and in increase in the surfactant molecular mass. In certain embodiments, the particles encapsulating antigens are manufactured by nanoprecipitation, co-precipitation, inert gas condensation, sputtering, microemulsion, sol-gel method, layer-by-layer technique or ionic gelation method. Several methods for manufacturing nanoparticles have been described in the literature and are incorporated herein by reference (Sánchez, Mejía, and Orozco 2020; Zielińska et al. 2020).

### Antigens

An antigen refers to a discreet portion of a molecule, such as a polypeptide or peptide sequence, a 3-D structural formation of a polypeptide or peptide, a polysaccharide or polynucleotide that can be recognized by a host immune cells. Antigen-specific refers to the ability of a subject's host cells to recognize and generate an immune response against an antigen alone, or to molecules that closely resemble the antigen, as with an epitope or mimotope.

“Anergy,” “tolerance,” or “antigen-specific tolerance” refers to insensitivity of T cells to T cell receptor-mediated stimulation. Such insensitivity is generally antigen-specific and persists after exposure to the antigenic peptide has ceased. For example, anergy in T cells is characterized by lack of cytokine production, e.g., IL-2. T-cell anergy occurs when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) results in failure to produce cytokines and subsequently failure to proliferate. Thus, a failure to produce cytokines prevents proliferation. Anergic T cells can, however, proliferate if cultured with cytokines (e.g., IL-2).

It is contemplated that the tolerizing therapy described herein is antigen-specific. For example, TIMPs administered as tolerizing therapy encapsulate one or more antigens associated with said tolerizing therapy and associated disease or condition being treated. It is contemplated that the TIMPs used in tolerizing therapy comprise one or more peanut antigens. The one or more peanut antigens are derived from peanut protein extract or may be peptides derived from known peanut proteins.

Over 15 peanut allergens are officially recognized by the WHO/IUIS Allergen Nomenclature Sub-Committee (www.allergen.org), Ara h1 to Ara h18, including Ara h1, Ara h2, Ara h3, Ara h5, Ara h6, Ara h7, Ara h8, Ara h9, Ara h10, Ara h11, Ara h12, Ara h13, Ara h14, Ara h15, Ara h16, Ara h17, and Ara h18. Peanut allergens can be classified into different groups based on their architecture (e.g., trimer, monomer, cupin, albumin, prolamin, profilin, oleosins, defensins, vincillin, and Nonspecific lipid transfer proteins (nsLTPs)) based on Ara h1, h2, h3, h5, h6 and h8, and each of these groups possesses a different degree of allergenic potency (Ozias-Akins et al., Allergy 74:888-898, 2019). Known peanut allergens include those derived from Arachis hypogaea Ara h1, Ara h2, Ara h3, Ara h5, Ara h6, Ara h7, Ara h8 and Ara h18. See e.g., UNIPROT Database No. E5G076 showing the Ara h1 polypeptide sequence (SEQ ID NO: 1), UNIPROT Database No. A0A445BYI5 for Ara h2 polypeptide (SEQ ID NO: 2), UNIPROT Database No. E5G077 for Ara h3 polypeptide (SEQ ID NO: 3) (see also UNIPROT Database No. 082580 (SEQ ID NO: 4) and Q9SQH7 (SEQ ID NO: 5) for Ara h3 isoallergens 1 and 2 (formerly Ara h4), respectively), UNIPROT Database No. L7QH52 for Ara h5 polypeptide (SEQ ID NO: 6), UNIPROT Database No. A5Z1 R0 for Ara h6 polypeptide (SEQ ID NO: 7), UNIPROT Database No. B4XID4 for Ara h7 polypeptide (SEQ ID NO: 8), UNIPROT Database No. Q6VT83 for Ara h8 polypeptide sequence (SEQ ID NO: 9), Ara h9, isoallergen1 and 2, UNIPROT Database No. B6CEX8 and B6CG41, (SEQ ID NO: 10 and 11) respectively; Ara h10, isoallergen 1 and 2, UNIPROT Database No. Q647G5 and Q647G4, (SEQ ID NO: 12 and 13) respectively; Ara h11, isoallergen 1 and 2, UNIPROT Database No. Q45W87 and Q45W86, (SEQ ID NO: 14 and 15) respectively; Ara h12 UNIPROT Database No. B3EWP3 (SEQ ID NO: 16); Ara h13, isoallergen 1 and 2, UNIPROT Database No. B3EWP4 and COHJZ1, (SEQ ID NO: 17 and 18) respectively; Ara h14, isoallergen 1, 2, and 3, UNIPROT Database No. Q9AX11, Q9AXI0 and Q6J1J8, (SEQ ID NO: 19-21) respectively; Ara h15, UNIPROT Database No. Q647G3 (SEQ ID NO: 22); Ara h16, UNIPROT Database No. AOA509ZX51 (SEQ ID NO: 23); Ara h17, UNIPROT A Database No. 0A510A9S3 (SEQ ID NO: 24); and Ara h18, UNIPROT Database No. A0A444XS96 (SEQ ID NO: 25).

In various embodiments, the allergenic peanut proteins include one or more of Ara h1, Ara h2, Ara h3, Ara h5, Ara h6, Ara h7, Ara h8, Ara h9, Ara h10, Ara h11, Ara h12, Ara h13, Ara h14, Ara h15, Ara h16, Ara h17, and Ara h18. In various embodiments, the peptides derived from peanut proteins comprise allergenic epitopes from one or more of Ara h1, Ara h2, Ara h3, Ara h5, Ara h6, Ara h7, Ara h8, Ara h9, Ara h10, Ara h11, Ara h12, Ara h13, Ara h14, Ara h15, Ara h16, Ara h17, and Ara h18 proteins.

In certain embodiments, one, two, three, or a higher number of antigens or antigenic peptides are used in the TIMPs. In certain embodiments, the one or more peanut antigens is encapsulated in the TIMP by covalent linkage to the interior surface of the particle (See e.g., US Patent Publication US20190282707, herein incorporated by reference). In certain embodiments, it is contemplated that sequences of two or more peanut proteins, e.g., from Ara h1, Ara h2, Ara h3, Ara h5, Ara h6, Ara h7, and/or Ara h8, are linked in a fusion protein and encapsulated within a TIMP described herein. In certain embodiments, it is contemplated that sequences of two or more peanut proteins, e.g., from Ara h1, Ara h2, Ara h3, Ara h5, Ara h6, Ara h7, Ara h8, Ara h9, Ara h10, Ara h11, Ara h12, Ara h13, Ara h14, Ara h15, Ara h16, Ara h17, and Ara h18, are linked in a fusion protein and encapsulated within a TIMP described herein. Methods for making TIMP with linked epitopes are described in US Patent Publication US20190365656, herein incorporated by reference.

### Methods of Use

Peanut allergy is the most common food allergy in the United States with up to 0.6% of adults and 0.8% of children being diagnosed. Additionally, peanut allergy is the most common cause of fatal food-related anaphylaxis due to its high prevalence in the population and the widespread use of peanut derived products as filler in many packaged foods. As a result, peanut allergy creates a significant health hazard in the United States.

Four major peanut allergenic proteins are Ara h1, Ara h2, Ara h3, and Ara h6. Ara h1 and Ara h2 are recognized by more than 95% of patients with peanut allergy.

Peanut avoidance by dietary modification has long been the most effective treatment for peanut allergy. However, severe cases of peanut allergy can additionally be triggered by foods processed near peanuts and/or by inhaling in the vicinity of peanut products. When complete avoidance of both ingesting peanut products and of areas contaminated with peanut products is not achieved, severe allergic reactions may occur requiring patients to receive medications such as epinephrine, antihistamines, and/or oral steroids.

Therapies currently approved for the treatment of peanut allergy (like PALFORZIA™) and those in clinical trials are only designed to protect against accidental peanut exposure by desensitizing the subject. The treatment regimen with PALFORZIA™ also includes a rather extensive daily dose escalation and maintenance schedule and may present challenges for subject compliance and still requires subjects to maintain peanut-free diet.

Therapeutic approaches rendering T cells tolerant to peanut protein could potentially cure peanut allergy thus eliminating the burdens associated with strict avoidance of peanut products.

Provided herein is a method of treating Peanut Allergy in a subject comprising administering to the subject TIMP-PPE, wherein TIMP-PPE is administered at a dose level determined based on the subject's weight. It is also contemplated that TIMP-PPE may be administered at a fixed dosage irrespective of the subject's weight. Provided herein is a method of treating Peanut Allergy in a subject comprising administering to the subject TIMP-PPE, wherein TIMP-PPE is administered at a dose of 0.1 to 12 mg/kg. Provided herein is a method of treating Peanut Allergy in a subject comprising administering to the subject TIMP-PPE, wherein TIMP-PPE is administered at a dose of 0.001 to 12 mg/kg. In various embodiments, contemplated is a method of treating peanut allergy in a subject comprising administering to the subject, TIMP-PPE, wherein TIMP-PPE is administered at a dose of 0.001 to 12 mg/kg based on the subject's weight or at a fixed dose between 0.1 mg and 800 mg. Also provided herein is a method of reducing an allergic immune response to peanut antigens in a subject suffering from PA comprising administering to the subject TIMP-PPE, wherein TIMP-PPE is administered at a dose of 0.001 to 12 mg/kg. Further contemplated herein is a method of reducing an allergic immune response to peanut antigens in a subject suffering from PA comprising administering to the subject TIMP-PPE, wherein TIMP-PPE is administered at a dose of 0.001 to 12 mg/kg based on the subject's weight or at a fixed dose between 0.1 mg and 800 mg.

Also contemplated, the TIMP-PPE is administered at a dose from about 0.001 to 10 mg/kg, from about 0.005 to 12 mg/kg, from about 0.01 to 12 mg/kg, from about 0.05 to 12 mg/kg, from about 0.1 to 12 mg/kg, from about 0.5 to 10 mg/kg, from about 1 to 8 mg/kg, from about 1.5 to 10 mg/kg, from about 2 to 12 mg/kg, from about 2 to 10 mg/kg, from about 3 to 10 mg/kg, from about 4 to 10 mg/kg, from about 4 to 12 mg/kg, or from about 5 to 12 mg/kg. Optionally, the TIMP-PPE is administered in a dose of about 0.001 mg/kg, 0.0025 mg/kg, 0.005 mg/kg, 0.01 mg/kg, 0.025 mg/kg, 0.05 mg/kg, 0.1 mg/kg, 0.25, 0.5 mg/kg, 1.0 mg/kg, 2.0 mg/kg, 4.0 mg/kg, 6 mg/kg, 8.0 mg/kg, 10 mg/kg, or 12 mg/kg. Alternatively, TIMP-PPE is administered at a dose of about 0.1 mg, 0.25 mg, 0.5 mg, 1 mg, 2 mg, 2.5 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg. In another embodiment, TIMP-PPE is administered at a concentration of between about 0.0005 mg/mL and about 50 mg/mL, optionally about 0.0005 mg/mL, 0.001 mg/mL, 0.005 mg/mL, 0.01 mg/mL, 0.05 mg/mL, 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL.

It is contemplated that the TIMP-PPE is administered in a single dose or in multiple doses. In various embodiments, TIMP-PPE is administered once weekly, once every two weeks, once every three weeks, once every 4 weeks, once every two months, once every three months, once every 6 months, or once per year. In certain embodiments, TIMP-PPE is administered in two doses one-week apart.

In various embodiments, TIMP-PPE is administered intravenously, subcutaneously, intramuscularly, intraperitoneally, intranasally, or orally. It is contemplated that if TIMP-PPE is given intravenously, it can be via intravenous infusion lasting about 1, 2, 3, 4, 5, 6, 7, 8, 10, 12, 18 or 20 hours, or via intravenous infusion lasting about 0.5, 1, 2, 3, 4, 5, 6, 7, 8, 10, 12, 18 or 20 hours.

Also provided herein is a method of enhancing the duration or potency of tolerance induced to peanut allergens by TIMP-PPE treatment comprising administering a booster dose of TIMP-PPE. In various embodiments, a booster dose of TIMP-PPE is administered in a single dose or in multiple doses following the original or initial TIMP-PPE administration. In various embodiments, the booster dose of TIMP-PPE is administered once weekly, once every two weeks, once every three weeks, once every 4 weeks, once every two months, once every three months, once every 6 months, or once per year. In various embodiments, the booster dose of TIMP-PPE is administered as a single dose every three months.

It is further contemplated that TIMP-PPE is administered alone or in combination with one or more additional therapeutics. Exemplary additional therapeutics include, but are not limited to, inhibitors of IgE, inhibitors of basophil activation, inhibitors of mast cell activation, an antihistamine, nonsteroid anti-inflammatory drug (NSAID), or a small molecule or biological therapeutic.

In various embodiments, TIMP-PPE is administered alone or in combination with one or more therapeutics useful in treating peanut allergy. Exemplary therapeutics include, but are not limited to, inhibitors of IgE, inhibitors of basophil activation, inhibitors of mast cell activation, an antihistamine, nonsteroid anti-inflammatory drug (NSAID), a prebiotic, a probiotic, histone deacetylase inhibitor, short chain fatty acids (e.g. acetate, butyrate, propionate, butyrate polymer), competitor of IgE for allergen binding sites, a cytokine inhibitor, microbiome therapy, a steroid, a corticosteroid, a leukotriene modifier, or a small molecule or biological therapeutic. Exemplary additional therapeutics or therapeutics useful in treating peanut allergy also include those that increase the number, frequency or activity of Tregs.

In various embodiments, the biologic is an antibody. In various embodiments, the antibody is an anti-IgE, anti-IL-4Rα, anti-IL-13, or an anti-IL-33 antibody. In various embodiments, the anti-IgE antibody is omalizumab (XOLAIR®). Exemplary anti-IL IL-4Rα antibodies include dupilumab (DUPIXENT®), and anti-IL-33 antibody includes etokinumab.

In various embodiments, the antihistamine is a first generation antihistamine. In various embodiments, the antihistamine is a second generation antihistamine. In various embodiments, the antihistamines are selected from the group consisting of brompheniramine, carbinoxamine maleate, chlorpheniramine, clemastine, diphenhydramine, hydroxyzine, triprolidine, azelastine, cetirizine, desloratadine, fexofenadine, levocetrizine, loratadine, and olopatadine. In various embodiments, the additional therapeutic is a steroid. In various embodiments, the steroid is selected from the group consisting of beclomethasone, ciclesonide, fluticasone furoate, mometasone, budenoside, fluticasone, triamcinolone, and loteprednol. In various embodiments, the additional therapeutic is a corticosteroid. In various embodiments, the corticosteroid is selected from the group consisting of cortisone, prednisone, prednisolone, methylprednisolone, dexamethasone, betamethasone, and hydrocortisone.

In various embodiments, the additional therapeutic is a nonsteroid anti-inflammatory drug (NSAID). In various embodiments, the therapeutic useful to treat peanut allergy is a nonsteroid anti-inflammatory drug (NSAID). In various embodiments the NSAID is a non-selective NSAID. In various embodiments, the NSAID is a selective NSAID. In various embodiments the NSAID is a COX-2 selective NSAID. In various embodiments the NSAID is a COX-1 selective NSAID. In various embodiments the NSAID is a prostaglandin synthase inhibitor. In various embodiments, the NSAID is selected from the group consisting of diclofenac, diclofenac potassium, diclofenac sodium, diflunisal, etodolac, flurbiprofen, fenoprofen, fenoprofen calcium, ketorolac, ketorolac tromethamine, ketoprofen, tolmetin, tolmetin sodium, aspirin, ibuprofen, naproxen, indomethacin, indomethacin sodium, sulindac, felbinac, piroxicam, mefenamic acid, meclofenamate sodium, meloxicam, nabumetone, oxaprozin, piroxicam, celecoxib, etodolac, etoricoxib, lumiracoxib, rofecoxib, and valdecoxib.

In various embodiments, the additional therapeutic is a leukotriene modifier. In various embodiments, the therapeutic useful to treat peanut allergy is a leukotriene modifier. In various embodiments the leukotriene modifier is an antileukotriene. In various embodiments the leukotriene modifier is a leukotriene receptor antagonist. In various embodiments the leukotriene modifier is a leukotriene synthesis inhibitor. In various embodiments the leukotriene modifier is selected from the group consisting of montelukast, zileuton, and zafirlukast.

In various embodiments, the additional therapeutic is administered prior to, during, or after the administration of TIMP-PPE. In various embodiments, the therapeutic useful in treating peanut allergy is administered prior to, during, or after the administration of TIMP-PPE.

In various embodiments, the additional therapeutic is administered intravenously, subcutaneously, intramuscularly, intraperitoneally, intranasally, via inhalation or orally. In various embodiments, the therapeutic useful in treating peanut allergy is administered intravenously, subcutaneously, intramuscularly, intraperitoneally, intranasally, via inhalation or orally.

The present disclosure provides a method of treating peanut allergy in a subject comprising administering to the subject TIMP-PPE in combination with an anti-IgE antibody, wherein TIMP-PPE is administered at a dose of about 0.001 mg/kg to 12 mg/kg, and wherein the anti-IgE antibody is administered at a dose of about 10 mg to about 500 mg. In various embodiments, the anti-IgE antibody is Omalizumab (XOLAIR®), Quilizumab or ligelizumab.

In various embodiments, the anti-IgE antibody administered in combination with TIMP-PPE is administered at a dose of about 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, or 500 mg. The dose level of the anti-IgE antibody is determined based on serum IgE levels, which can be between about 30-100 IU/mL, 100-200 IU/mL, 200-300 IU/mL, 300-400 IU/mL, 400-500 IU/mL, 500-600 IU/mL, 600-700 IU/mL, 700-800 IU/mL, 800-900 IU/mL, 900-1000 IU/mL, 1000-1100 IU/mL, 1100-1200 IU/mL, 1200-1300 IU/mL, 1300-1400 IU/mL, or between 1400-1500 IU/mL. Alternatively, the dose level of the anti-IgE antibody is determined based on the weight of the subject. In various embodiments, the weight of the subject is between 30-40 kg, 40-50 kg, 50-60 kg, 60-70 kg, 70-80 kg, 80-90 kg, 90-125 kg, or between 125-150 kg.

In various embodiments, the anti-IgE antibody is administered in a single dose or in multiple doses. In various embodiments, the anti-IgE antibody is administered once weekly, once every two weeks, once every three weeks, or once every four weeks. In various embodiments, the anti-IgE antibody is administered prior to, concomitantly, or subsequent to/after the administration of TIMP-PPE. In various embodiments, the anti-IgE antibody is administered for one week, two weeks, three weeks, or four weeks prior to the administration of TIMP-PPE. In various embodiments, the anti-IgE antibody is administered for one week, two weeks, three weeks, or four weeks after the administration of TIMP-PPE.

Concomitant or concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks. It is further contemplated that the therapeutics are administered in a separate formulation and administered concurrently or concomitantly, with concurrently referring to agents given within 30 minutes of each other. Prior administration refers to administration of a therapeutic within the range of one week prior to treatment with TIMP-PPE, up to 30 minutes before administration of TIMP-PPE. Subsequent administration is meant to describe administration from 30 minutes after TIMP-PPE treatment up to one week after TIMP-PPE administration.

The present disclosure provides methods for treating peanut allergy comprising administering to a subject TIMP-PPE in combination with anti-IgE antibody, wherein the subject is administered anti-IgE antibody once weekly for two weeks or once weekly for four weeks prior to the administration of TIMP-PPE, wherein TIMP-PPE is administered in two doses one-week apart at a dose level of between 0.001 mg/kg and 12 mg/kg, and wherein the anti-IgE antibody is administered at a dose level of between about 50 mg and 500 mg.

Also provided is a method of treating peanut allergy in a subject comprising administering to the subject TIMP-PPE in combination with an anti-IL-4Rα antibody, wherein TIMP-PPE is administered at a dose of about 0.001 mg/kg to 12 mg/kg, and wherein the anti-IL-4Rα antibody is administered at a dose of about 10 mg to about 500 mg. In various embodiments, the anti-IL-4Rα antibody is dupilumab (DUPIXENT®).

In various embodiments, the anti-IL-4Rα antibody is administered at a dose of about 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, 500 mg, 525 mg, 550 mg, 575 mg, or 600 mg. In various embodiments, the dose level of the anti-IL-4Rα antibody is determined based on serum IgE levels, which can be between about 30-100 IU/mL, 100-200 IU/mL, 200-300 IU/mL, 300-400 IU/mL, 400-500 IU/mL, 500-600 IU/mL, 600-700 IU/mL, 700-800 IU/mL, 800-900 IU/mL, 900-1000 IU/mL, 1000-1100 IU/mL, 1100-1200 IU/mL, 1200-1300 IU/mL, 1300-1400 IU/mL, or between 1400-1500 IU/mL. Alternatively, the anti-IL-4Rα antibody dose level is determined based on the weight of the subject. In various embodiments, the weight of the subject is between 30-40 kg, 40-50 kg, 50-60 kg, 60-70 kg, 70-80 kg, 80-90 kg, 90-125 kg, or between 125-150 kg.

In various embodiments, the anti-IL-4Rα antibody is administered in a single dose or in multiple doses. In various embodiments, the anti-IL-4Rα antibody is administered once weekly, once every two weeks, once every three weeks, or once every four weeks. In various embodiments, the anti-IL-4Rα antibody is administered prior to, concomitantly, or after the administration of TIMP-PPE. In various embodiments, the anti-IL-4Rα antibody is administered for one week, two weeks, three weeks, or four weeks prior to the administration of TIMP-PPE. In various embodiments, the anti-IL-4Rα antibody is administered for one week, two weeks, three weeks, or four weeks after the administration of TIMP-PPE. In various embodiments, the anti-IL-4Rα antibody is administered at an initial dose level of between 400 mg and 600 mg for two doses followed by a maintenance dose level of between 200 mg and 300 mg for subsequent doses.

In various embodiments, the antihistamine administered in combination with TIMP-PPE is administered at a dose of about 0.05 mg to 2000 mg. In various embodiments, the antihistamine is administered at a dose of about 0.05 mg, 0.1 mg, 0.5 mg, 1 mg, 2 mg, 4 mg, 5 mg, 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, 500 mg, 1000 mg, 1500 mg, or 2000 mg. In various embodiments, the antihistamine is administered in a single dose or in multiple doses. In various embodiments, the antihistamine is administered once daily, once weekly, once every two weeks, once every three weeks, or once every four weeks. In various embodiments, the antihistamine is administered two time, three times, four times, five times, or six times daily. In various embodiments, the antihistamine is administered prior to, concomitantly, or after the administration of TIMP-PPE. In various embodiments, the antihistamine is administered for one week, two weeks, three weeks, four weeks, 1 month, 2 months, 3 months, 4 months, 5 months, 6 months, 7 months, 8 months, 9 months, 10 months, 11 months, or 12 months prior to the administration of TIMP-PPE. In various embodiments, the antihistamine is administered for one week, two weeks, three weeks, four weeks, 1 month, 2 months, 3 months, 4 months, 5 months, 6 months, 7 months, 8 months, 9 months, 10 months, 11 months, or 12 months after the administration of TIMP-PPE. In various embodiments, the antihistamine is administered 5, 10, 15, 30, 45, or 60 minutes prior to the administration of TIMP-PPE. In various embodiments, the antihistamine is administered 5, 10, 15, 30, 45, or 60 minutes after the administration of TIMP-PPE. In various embodiments, the antihistamine is administered 1, 2, 3, 4, 5, 6, 7, 8, 9, 10, 11, 12, 13, 14, 15, 16, 17, 18, 19, 20, 21, 22, 23, or 24 hours prior to the administration of TIMP-PPE. In various embodiments, the antihistamine is administered 1, 2, 3, 4, 5, 6, 7, 8, 9, 10, 11, 12, 13, 14, 15, 16, 17, 18, 19, 20, 21, 22, 23, or 24 hours after the administration of TIMP-PPE. In various embodiments, the antihistamine is administered 1, 2, 3, 4, 5, 6, or 7 days prior to the administration of TIMP-PPE. In various embodiments, the antihistamine is administered 1, 2, 3, 4, 5, 6, or 7 days after the administration of TIMP-PPE. In various embodiments, the antihistamine is a first generation antihistamine or a second generation antihistamine. In various embodiments, the antihistamines are selected from the group consisting of brompheniramine, carbinoxamine maleate, chlorpheniramine, clemastine, diphenhydramine, hydroxyzine, triprolidine, azelastine, cetirizine, desloratadine, fexofenadine, levocetrizine, doxylamine, ebastine, embramine, epinephrine, fexofenadine, loratadine, and olopatadine.

In various embodiments, the steroid administered in combination with TIMP-PPE is administered at a dose of about 0.05 mg to 2000 mg. In various embodiments, the steroid is administered at a dose of about 0.05 mg, 0.1 mg, 0.5 mg, 1 mg, 2 mg, 4 mg, 5 mg, 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, 500 mg, 1000 mg, 1500 mg, or 2000 mg. In various embodiments, the steroid is administered in a single dose or in multiple doses. In various embodiments, the steroid is administered once daily, once weekly, once every two weeks, once every three weeks, or once every four weeks. In various embodiments, the steroid is administered two times, three times, four times, five times, or six times daily. In various embodiments, the steroid is administered prior to, concomitantly, or after the administration of TIMP-PPE. In various embodiments, the steroid is administered for one week, two weeks, three weeks, four weeks, 1 month, 2 months, 3 months, 4 months, 5 months, 6 months, 7 months, 8 months, 9 months, 10 months, 11 months, or 12 months prior to the administration of TIMP-PPE. In various embodiments, the steroid is administered for one week, two weeks, three weeks, four weeks, 1 month, 2 months, 3 months, 4 months, 5 months, 6 months, 7 months, 8 months, 9 months, 10 months, 11 months, or 12 months after the administration of TIMP-PPE. In various embodiments, the steroid is administered 5, 10, 15, 30, 45, or 60 minutes prior to the administration of TIMP-PPE. In various embodiments, the steroid is administered 5, 10, 15, 30, 45, or 60 minutes after the administration of TIMP-PPE. In various embodiments, the steroid is administered 1, 2, 3, 4, 5, 6, 7, 8, 9, 10, 11, 12, 13, 14, 15, 16, 17, 18, 19, 20, 21, 22, 23, or 24 hours prior to the administration of TIMP-PPE. In various embodiments, the steroid is administered 1, 2, 3, 4, 5, 6, 7, 8, 9, 10, 11, 12, 13, 14, 15, 16, 17, 18, 19, 20, 21, 22, 23, or 24 hours after the administration of TIMP-PPE. In various embodiments, the steroid is administered 1, 2, 3, 4, 5, 6, or 7 days prior to the administration of TIMP-PPE. In various embodiments, the steroid is administered 1, 2, 3, 4, 5, 6, or 7 days after the administration of TIMP-PPE. In various embodiments, the steroid is selected from the group consisting of beclomethasone, ciclesonide, fluticasone furoate, mometasone, budenoside, fluticasone, triamcinolone, and loteprednol.

In various embodiments, the additional therapeutic is a corticosteroid. In various embodiments, the therapeutic useful to treat peanut allergy is a corticosteroid. In various embodiments, the corticosteroid administered in combination with TIMP-PPE is administered at a dose of about 0.05 mg to 2000 mg. In various embodiments, the corticosteroid is administered at a dose of about 0.05 mg, 0.1 mg, 0.5 mg, 1 mg, 2 mg, 4 mg, 5 mg, 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, 500 mg, 1000 mg, 1500 mg, or 2000 mg. In various embodiments, the corticosteroid is administered in a single dose or in multiple doses. In various embodiments, the corticosteroid is administered once daily, once weekly, once every two weeks, once every three weeks, or once every four weeks. In various embodiments, the corticosteroid is administered two times, three times, four times, five times, or six times daily. In various embodiments, the corticosteroid is administered prior to, concomitantly, or after the administration of TIMP-PPE. In various embodiments, the corticosteroid is administered for one week, two weeks, three weeks, four weeks, 1 month, 2 months, 3 months, 4 months, 5 months, 6 months, 7 months, 8 months, 9 months, 10 months, 11 months, or 12 months prior to the administration of TIMP-PPE. In various embodiments, the corticosteroid is administered for one week, two weeks, three weeks, four weeks, 1 month, 2 months, 3 months, 4 months, 5 months, 6 months, 7 months, 8 months, 9 months, 10 months, 11 months, or 12 months after the administration of TIMP-PPE. In various embodiments, the corticosteroid is administered 5, 10, 15, 30, 45, or 60 minutes prior to the administration of TIMP-PPE. In various embodiments, the corticosteroid is administered 5, 10, 15, 30, 45, or 60 minutes after the administration of TIMP-PPE. In various embodiments, the corticosteroid is administered 1, 2, 3, 4, 5, 6, 7, 8, 9, 10, 11, 12, 13, 14, 15, 16, 17, 18, 19, 20, 21, 22, 23, or 24 hours prior to the administration of TIMP-PPE. In various embodiments, the corticosteroid is administered 1, 2, 3, 4, 5, 6, 7, 8, 9, 10, 11, 12, 13, 14, 15, 16, 17, 18, 19, 20, 21, 22, 23, or 24 hours after the administration of TIMP-PPE. In various embodiments, the corticosteroid is administered 1, 2, 3, 4, 5, 6, or 7 days prior to the administration of TIMP-PPE. In various embodiments, the corticosteroid is administered 1, 2, 3, 4, 5, 6, or 7 days after the administration of TIMP-PPE. In various embodiments, the corticosteroid is selected from the group consisting of cortisone, prednisone, prednisolone, methylprednisolone, dexamethasone, betamethasone, and hydrocortisone.

In various embodiments, the NSAID administered in combination with TIMP-PPE is administered at a dose of about 0.05 mg to 2000 mg. In various embodiments, the NSAID is administered at a dose of about 0.05 mg, 0.1 mg, 0.5 mg, 1 mg, 2 mg, 4 mg, 5 mg, 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, 500 mg, 1000 mg, 1500 mg, or 2000 mg. In various embodiments, the NSAID is administered in a single dose or in multiple doses. In various embodiments, the NSAID is administered once daily, once weekly, once every two weeks, once every three weeks, or once every four weeks. In various embodiments, the NSAID is administered two times, three times, four times, five times, or six times daily. In various embodiments, the NSAID is administered prior to, concomitantly, or after the administration of TIMP-PPE. In various embodiments, the NSAID is administered for one week, two weeks, three weeks, four weeks, 1 month, 2 months, 3 months, 4 months, 5 months, 6 months, 7 months, 8 months, 9 months, 10 months, 11 months, or 12 months prior to the administration of TIMP-PPE. In various embodiments, the NSAID is administered for one week, two weeks, three weeks, four weeks, 1 month, 2 months, 3 months, 4 months, 5 months, 6 months, 7 months, 8 months, 9 months, 10 months, 11 months, or 12 months after the administration of TIMP-PPE. In various embodiments, the NSAID is administered 5, 10, 15, 30, 45, or 60 minutes prior to the administration of TIMP-PPE. In various embodiments, the NSAID is administered 5, 10, 15, 30, 45, or 60 minutes after the administration of TIMP-PPE. In various embodiments, the NSAID is administered 1, 2, 3, 4, 5, 6, 7, 8, 9, 10, 11, 12, 13, 14, 15, 16, 17, 18, 19, 20, 21, 22, 23, 24, 36, 48, 72, or 96 hours prior to the administration of TIMP-PPE. In various embodiments, the NSAID is administered 1, 2, 3, 4, 5, 6, 7, 8, 9, 10, 11, 12, 13, 14, 15, 16, 17, 18, 19, 20, 21, 22, 23, 24, 36, 48, 72, or 96 hours after the administration of TIMP-PPE. In various embodiments, the NSAID is administered 1, 2, 3, 4, 5, 6, or 7 days prior to the administration of TIMP-PPE. In various embodiments, the NSAID is administered 1, 2, 3, 4, 5, 6, or 7 days after the administration of TIMP-PPE. In various embodiments the NSAID is a non-selective NSAID, a COX-2 selective NSAID or a COX-1 selective NSAID. In various embodiments the NSAID is a prostaglandin synthase inhibitor. In various embodiments, the NSAID is selected from the group consisting of diclofenac, diclofenac potassium, diclofenac sodium, diflunisal, etodolac, flurbiprofen, fenoprofen, fenoprofen calcium, ketorolac, ketorolac tromethamine, ketoprofen, tolmetin, tolmetin sodium, acetylsalicylic acid, aspirin, ibuprofen, naproxen, indomethacin, indomethacin sodium, sulindac, felbinac, piroxicam, mefenamic acid, meclofenamate sodium, meloxicam, nabumetone, oxaprozin, piroxicam, celecoxib, etodolac, etoricoxib, lumiracoxib, rofecoxib, valdecoxib.

In various embodiments, the leukotriene modifier administered in combination with TIMP-PPE is administered at a dose of about 0.05 mg to 2000 mg. In various embodiments, the leukotriene modifier is administered at a dose of about 0.05 mg, 0.1 mg, 0.5 mg, 1 mg, 2 mg, 4 mg, 5 mg, 10 mg, 25 mg, 50 mg, 75 mg, 100 mg, 125 mg, 150 mg, 175 mg, 200 mg, 225 mg, 250 mg, 275 mg, 300 mg, 375 mg, 400 mg, 425 mg, 450 mg, 475 mg, 500 mg, 1000 mg, 1500 mg, or 2000 mg. In various embodiments, the leukotriene modifier is administered in a single dose or in multiple doses. In various embodiments, the leukotriene modifier is administered once daily, once weekly, once every two weeks, once every three weeks, or once every four weeks. In various embodiments, the leukotriene modifier is administered two times, three times, four times, five times, or six times daily. In various embodiments, the leukotriene modifier is administered prior to, concomitantly, or after the administration of TIMP-PPE. In various embodiments, the leukotriene modifier is administered for one week, two weeks, three weeks, four weeks, 1 month, 2 months, 3 months, 4 months, 5 months, 6 months, 7 months, 8 months, 9 months, 10 months, 11 months, or 12 months prior to the administration of TIMP-PPE. In various embodiments, the leukotriene modifier is administered for one week, two weeks, three weeks, four weeks, 1 month, 2 months, 3 months, 4 months, 5 months, 6 months, 7 months, 8 months, 9 months, 10 months, 11 months, or 12 months after the administration of TIMP-PPE. In various embodiments, the leukotriene modifier is administered 5, 10, 15, 30, 45, or 60 minutes prior to the administration of TIMP-PPE. In various embodiments, the leukotriene modifier is administered 5, 10, 15, 30, 45, or 60 minutes after the administration of TIMP-PPE. In various embodiments, the leukotriene modifier is administered 1, 2, 3, 4, 5, 6, 7, 8, 9, 10, 11, 12, 13, 14, 15, 16, 17, 18, 19, 20, 21, 22, 23, 24, 36, 48, 72, or 96 hours prior to the administration of TIMP-PPE. In various embodiments, the leukotriene modifier is administered 1, 2, 3, 4, 5, 6, 7, 8, 9, 10, 11, 12, 13, 14, 15, 16, 17, 18, 19, 20, 21, 22, 23, 24, 36, 48, 72, or 96 hours after the administration of TIMP-PPE. In various embodiments, the leukotriene modifier is administered 1, 2, 3, 4, 5, 6, or 7 days prior to the administration of TIMP-PPE. In various embodiments, the leukotriene modifier is administered 1, 2, 3, 4, 5, 6, or 7 days after the administration of TIMP-PPE. In various embodiments the leukotriene modifier is an antileukotriene, a leukotriene receptor antagonist, or a leukotriene synthesis inhibitor. In various embodiments the leukotriene modifier is selected from the group consisting of montelukast, zileuton, and zafirlukast.

It is provided that administering TIMP-PPE to a subject in need thereof, alone or in combination with one or more additional therapeutics, relieves one or more symptoms of peanut allergy. Symptoms of peanut allergy include skin reactions, hives, skin redness, skin swelling, itching, tightening of the throat, difficulty breathing, shortness of breath, and anaphylaxis. Additional symptoms include digestive problems such as diarrhea, stomach cramps, nausea, or vomiting, and drop in blood pressure.

It is provided that administering TIMP-PPE to a subject in need thereof, alone or in combination with one or more therapeutics useful in treating peanut allergy, relieves one or more symptoms of peanut allergy. Symptoms of peanut allergy include skin reactions, hives, skin redness, skin swelling, itching, tightening of the throat, difficulty breathing, shortness of breath, digestive problems such as diarrhea, stomach cramps, nausea, or vomiting, drop in blood pressure, and anaphylaxis.

It is also contemplated that administering TIMP-PPE to a subject in need thereof, alone or in combination one or more additional therapeutics, reduces the duration and severity of an allergic immune response to peanut proteins or following exposure to peanut proteins. An allergic immune response contemplated herein includes a Th2 T cell response, B-cell activation, basophil activation, eosinophil activation, mast cell activation, and/or IgE induction. Additional immune responses include a T cell dependent mechanism involving the upregulation of T helper type-2 (Th2) cytokine production (e.g., IL-4, IL-5, IL-9, and IL-13), B cell class switching leading to the production of IgE antibody, and/or IgE:IgG ratio.

It is also contemplated that administering TIMP-PPE to a subject in need thereof, alone or in combination one or more therapeutics useful in treating peanut allergy, reduces the duration and severity of an allergic immune response to peanut proteins or following exposure to peanut proteins. An allergic immune response contemplated herein includes a T cell dependent mechanism involving the upregulation of T helper type-2 (Th2) cytokine production (e.g., IL-4, IL-5, IL-9, and IL-13,), B-cell activation, B cell class switching leading to the production of IgE antibody, basophil activation, eosinophil activation, mast cell activation, and/or IgE:IgG ratio.

### Screening Methods

It is contemplated that induction of and maintenance of immunological tolerance is monitored in a subject suffering from peanut allergy treated, or about to undergo treatment, with antigen-specific tolerizing therapy consisting of TIMPs encapsulating peanut allergens as described herein.

Methods of screening for cell types, cytokines or other measures of tolerance from a subject undergoing tolerizing therapy as described herein are known in the art. Methods of assessing tolerance are done using such techniques as flow cytometry, Mass Cytometry (CyTOF), ELISA, ELISPOT, in vitro/ex vivo cell stimulation assays (including, but not limited to, cell proliferation assays, basophil activation test (BAT), macrophage stimulation assays), measuring autoantibodies or measuring Ig serotype, e.g., by ImmunoCap assay.

In various embodiments, immune tolerance status of a subject is determined from the assay of one or more biological samples from the subject. Biological samples include whole-blood, peripheral blood, peripheral blood mononuclear cells (PBMCs), serum, plasma, urine, cerebrospinal fluid (CSF), stool, a tissue biopsy, and/or a bone-marrow biopsy. In various embodiments, the assay of the biological sample(s) includes analyzing levels of, and or presence or absence of, cell-surface proteins, extracellular proteins, intracellular proteins, nucleic acids, metabolites, and/or combinations thereof.

Cells assayed from the biological sample include immune cells, non-immune cells, and/or combinations thereof. Immune cells include innate immune cells, adaptive immune cells, and/or combinations thereof. Innate immune cells assayed from the biological sample(s) include antigen-presenting cells (APCs). Exemplary innate immune cells assayed from the biological sample include monocytes, macrophages, neutrophils, granulocytes, dendritic cells, mast cells, eosinophils, basophils, and/or combinations thereof. Adaptive immune cells assayed from the biological sample(s) include effector immune cells, such as CD4+ T-cells, CD8+ T-cells, B cells, NK cells, NK-T cells, and/or combinations thereof. In various embodiments, the T cells are Th1 cells, Th2a cells, Treg cells, and Tr cells.

In certain embodiments, the cells assayed from the biological sample(s) are epithelial cells, stromal cells, endothelial cells, fibroblasts, pericytes, adipocytes, mesenchymal stem cells, hematopoietic stem cells, hematopoietic progenitor cells, liver sinusoidal endothelial cells (LSECs), and/or Kupffer cells.

One aspect of a subject's immune tolerance status, and immune signature, is determined by analyzing one or more proteins from one or more biological sample(s) from the subject. In various embodiments, the proteins are cytokines and/or chemokines. In various embodiments the proteins are cell signaling proteins. In various embodiments, the cytokines and chemokines are selected from the group consisting of IL-1a, IL-1p, IL-2, IL-3, IL-4, IL-5, IL-6, IL-7, IL-8, IL-9, IL-10, IL-11, IL-12, IL-12p70, IL-13, IL-14, IL-15, IL-16, IL-17, IL-17, IL-18, IL-20, IL-21, IL-22, IL-23, IL-24, IL-25, IL-26, IL-27, IL-27b, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1α, CXCL4 (MIP-1β, CXCL5 (RANTES), CXCL6, CXCL7, CXCL8, CXCL9, CXCL10, CXCL11, CXCL12, CXCL13, CXCL14, CXCL15, CXCL16, CXCL17, GM-CSF, IFN-α, IFN-β, IFN-γ, TNF-α, TGF-β1, TGF-β2, TGF-β3, soluble CD14, and/or combinations thereof.

In various embodiments, the protein is a protease. In various embodiments, the protease is an aspartic protease, a cysteine protease, a metalloprotease, a serine protease, and/or a threonine protease. In various embodiments, the protease is selected from the group consisting of ADAM1, ADAM2, ADAM7, ADAM8, ADAM9, ADAM10, ADAM11, ADAM12, ADAM15, ADAM17, ADAM18, ADAM19, ADAM20, ADAM21, ADAM22, ADAM23, ADAM28, ADAM29, ADAM30, ADAM33, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. In various embodiments, proteins associated with apoptosis are selected from the group consisting of P53, Caspase 1, Caspase 2, Caspase 3, Caspase 4, Caspase 5, Caspase 6, Caspase 7, Caspase 8, Caspase 9, Caspase 10, Caspase 11, Caspase 12, Caspase 13, Caspase 14, BCL-2, BCL-XL, MCL-1, CED-9, A1, BFL1, BAX, BAK, DIVA, BCL-XS, BIK, BIM, BAD, BID, and EGL-1. Several methods for assaying proteins from a biological sample have been described in the literature including enzyme-linked immunosorbent assay (ELISA), western blots, and mass spectrometry. In various embodiments the protein is one or more immunoglobulins (Ig). In various embodiments, the Ig are selected from the group consisting of IgA, IgD, IgE, IgM, and/or variants thereof. In various embodiments the immunoglobulins are antigen specific. Several methods for the detection of immunoglobulins from a biological sample have been described in the literature including ELISA and ImmunoCap

A list of human metabolites that can be assayed from a biological sample can be found in the literature including in (Psychogios et al., 2011), (Wishart et al., HMDB: the Human Metabolome Database. Nucleic Acids Res. 2007 January; 35 (Database issue):D521-6, 2007), and the Human Metabalome Database (HMDB) and are incorporated herein by reference.

One aspect of a subject's immune tolerance status, and immune signature, is determined by analyzing one or more cell-surface proteins from a biological sample(s). In various embodiments, the cell-surface proteins include CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD45, CD45RA, CD47, CD45RA, CD45RO, CD48, CD52, CD55, CD56, CD58, CD61, CD66b, CD69, CD70, CD72, CD79, CD68, CD84, CD86, CD93, CD94, CD95, CRACC, BLAME, BCMA, CD103, CD107, CD112, CD120a, CD120b, CD123, CD125, CD127, CD134, CD135, CD140a, CD141, CD154, CD155, CD160, CD161, CD163, CD172a, XCR1, CD203c, CD204, CD206, CD207 CD226, CD244, CD267, CD268, CD269, CD355, CD358, CRTH2, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2RB, IL-4R, IL-5Rα, CSF2RB, IL-6Rα, gp130, IL-7Rα, IL-9R, IL-10R, IL-12Rβ1, IL-12Rβ2, IL-13Rα1, IL-13Rα2, IL-15Rα, IL-21R, IL-23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, FAS, FAS-L, FC, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1 α,β, γ, δ, and ε, H60a, H60b, H60c, GPR15, ST2, and/or combinations thereof. Integrins include α1, α2, αIIb, α3, α4, α5, α6, α7, α8, α9, α10, α11, αD, αE, αL, αM, αV, αX, β1, β2, β3, β4, β5, β6, β7, β8 and/or combinations thereof. TCR include α, β, γ, δ, ε, ζ chains and/or combinations thereof. Several methods have been described in the literature for assaying of cell-surface protein expression, including Flow Cytometry and Mass Cytometry (CyTOF).

One aspect of a subject's immune tolerance status, and immune signature, is determined by analyzing one or more metabolites from the biological sample(s). In various embodiments, the metabolite is an inflammatory metabolite. In various embodiments, the metabolite is an anti-inflammatory metabolite. In various embodiments, examples of inflammatory metabolites include acids, lipids, sugars, amino acids, lactate, trimethylamine N-oxide, O-acetyl creatine, L-carnitine, choline, succinate, glutamine, fatty acids, cholesterol, 3-hydroxybutyrate, 3′-sialyllactose, arachidonic acid, prostaglandin (G2 and H2), PGD2, PGE2, PGF2a, PGI2, TXA2, leukotrienes (A4, B4, C4, D4, E4), kynurenine, 3-hydroxy kynurenine, lipoxin A4, and lipoxin B4. In various embodiments, examples of anti-inflammatory metabolites include 2-amino-3-carboxymuconic 6-semialdehyde, picolinic acid, anthranilic acid, 3-hydroxylanthranilic acid, glutaryl co-A, NAD+, quinolinic acid, arginine, butyrate, and adenosine. A list of human metabolites that can be assayed from a biological sample can be found in the literature including in (Psychogios et al., 2011 PLoS One 6(2):e16957), (Wishart et al., HMDB: the Human Metabolome Database. Nucleic Acids Res. 2007 January; 35 (Database issue):D521-6, 2007), and the Human Metabalome Database (HMDB) each of which is incorporated herein by reference.

In certain embodiments, the subject's tolerance status is determined by analyzing nucleic acids from the biological sample(s). In various embodiments, the nucleic acids are DNA and/or RNA, including, but not limited to, single stranded DNA, double stranded DNA, mRNA, rRNA, tRNA, siRNA, miRNA, long non-coding RNAs (long ncRNAs, lncRNA), and non-coding RNA (ncRNA), mitochondrial RNA. In various embodiments, the subject's immune tolerance status is determined by assaying gene expression from the biological sample(s). In various embodiments, the immune tolerance status is determined by assaying gene expression associated with immune function, an antibody, foreign body response, metabolism, apoptosis, cell death, necrosis, ferroptosis, autophagy, cell migration, endocytosis, phagocytosis, pinocytosis, tight-junction regulation, cell adhesion, differentiation, and/or combinations thereof. In various embodiments, the immune tolerance status is determined by assaying gene expression associated with immune suppression. In various embodiments, the immune tolerance status is determined by assaying gene expression associated with immune activation. In various embodiments, the immune tolerance status is determined by assaying gene expression associated with immune regulatory functions. In various embodiments, nucleic acid analysis is used to generate an immune tolerance signature. Several methodologies have been described in the literature for high-throughput gene expression analysis including RNA sequencing (RNA-seq), single-cell RNA sequencing (scRNA-seq), exome sequencing, and microarray-based analyses.

The biological sample is optionally assayed after in vivo and/or ex vivo stimulation with one or more stimuli such as an antigen, an allergen, and one or more activating agents. It is contemplated that the T cells, B cells, and immunoglobulins used in the assay are antigen specific. Exemplary T cells include effector memory T cells, antigen specific T cells, activated antigen specific T cells, Th1 cells, pathogenic Th2a+ cells, Th17 cells, T follicular helper (TFH) cells, THO cells, or other antigen-specific T cells. B cells include effector B cells, memory B cells, plasma cells, and regulatory B (Breg) cells. In certain embodiments, T cells or B cells are identified based on the expression of proteins described in Table 1.

TABLE 1

| Cell Type | Expression Marker |
|---|---|
| Effector memory (EM) T cell | CD45RA^{LO} CD4⁺ |
| Disease specific T cell | CD45RA^{LO} CD4⁺ CD154⁺ CD137⁺ |
| Activated disease specific T cell | CD38⁺ |
| Pathogenic Th2a+ cell | CD45RA^{LO} CD4⁺ CD154⁺ CD137⁺ CXCR5⁻ CRTH2^{+/-} CD27⁻ CD200R⁺ CD161⁺ |
| Th1 cell | CXCR5⁻ CRTH2⁻ CXCR3⁺ CCR6⁻ |
| Th17 cell | CXCR5⁻ CRTH2⁻ CXCR3⁻ CCR6⁺ |
| TFH cell | CD45RA^{LO} CD4⁺ CXCR5⁺ |
| Treg | CD45RA^{LO} CD4⁺ CD154⁻ CD127⁻ CD25⁺ TIGIT⁺ CD137⁺ GARP^{+/-} OX40^{+/-} |
| Th0 cell | CXCR5⁻ CRTH2⁻ CXCR3⁻ CCR6⁻ CCR4⁻ |
| Activated antigen specific CD8 T cell | CD8⁺ CD137⁺ CD69⁺ CD355⁺ |
| Gut homing T cell | CD4⁺ α4β7⁺ and CD8⁺ αEβ7⁺ |
| Brain homing T cells | CD45RO⁺ CCR7⁺ CD27⁺ and/or CD45RO⁺ CCR7⁺ CCR5⁺ CXCR3⁺ CD27⁺ |
| Exhausted T cells | CD4⁺ TIGIT⁺ KLRG1⁺ PD-1⁺ |
| Plasma blast B cells (effector B | CD3⁻CD19⁺CD137⁺CD45RA⁺CD27⁺38⁺ |
| cells) | |

In various embodiments, the immune tolerance status of the subject is determined by obtaining one or more samples, e.g., whole blood, from the subject pre-dose on the day of the first TIMP-PPE administration (Day 1), 14 days after administration of the second dose, and then at every 90 days post-second dose (e.g., Days 90, 180, 270, and 360 post-second dose). Whole blood can then be processed to isolate peripheral blood mononuclear cells (PBMCs), basophils, neutrophils, plasma, and serum for downstream analyses. Assay of cells isolated from one or more samples collected from the subject and analyzed using such methods as described below.

In various embodiments, the immune tolerance status of the subject is determined by obtaining one or more samples, e.g., whole blood, from the subject pre-dose on the day of the first TIMP-PPE administration (Day 1), 14 days after administration of the second dose (Day 15), at Day 60, and then, optionally, at every 90 days post-second dose (e.g., Days 90, 180, 270, and 360 post-second dose). Whole blood can then be processed to isolate peripheral blood mononuclear cells (PBMCs), basophils, neutrophils, plasma, and serum for downstream analyses. Assay of cells isolated from one or more samples collected from the subject and analyzed using such methods as described below.

In various embodiments, the immune tolerance status of the subject determined prior to administration of TIMP-PPE serves as the baseline. In various embodiments, the subject's baseline is determined from the assay of one or more biological samples 1, 2, 3, 4, 5, 6, or 7 days prior to administration of TIMP-PPE. In various embodiments, the subject's baseline is determined from the assay of one or more biological samples 1, 2, 3, or 4 weeks prior to administration of TIMP-PPE. In various embodiments, the subject's baseline is determined from the assay of one or more biological samples 1, 2, 3, 4, 5, 6, 7, 8, 9, 10, 11, or 12 months prior to administration of TIMP-PPE.

In various embodiments, the immune tolerance status of the subject is determined after administration of TIMP-PPE. In various embodiments, the immune tolerance status of the subject is determined from the assay of one or more biological samples 1, 2, 3, 4, 5, 6, or 7 days after administration of TIMP-PPE. In various embodiments, the immune tolerance is determined from the assay of one or more biological samples 1, 2, 3, or 4 weeks after the administration of TIMP-PPE. In various embodiments, the subject's immune tolerance is determined from the assay of one or more biological samples 1, 2, 3, 4, 5, 6, 7, 8, 9, 10, 11, or 12 months after the administration of TIMP-PPE. In various embodiments, the immune tolerance status of the subject determined after administration of TIMP-PPE is compared to the baseline. In various embodiments, the immune tolerance status of the subject determined after the administration of TIMP-PPE is compared to a healthy subject or subject administered placebo.

In various embodiments, the immune tolerance status of the subject is determined after administration of TIMP-PPE whether it is the initial or original dose of TIMP-PPE or a booster dose of TIMP-PPE.

The following analyses are contemplated to follow the immune status and tolerance induction of a subject receiving TIMP-PPE tolerizing therapy.

The proportion of peanut-specific Th2a+ cells (Th2a+ cells/total peanut specific T cells) stimulated ex vivo with purified antigenic peanut proteins, can be measured for example, by flow cytometry. Th2a+ cells are defined as CRTH2+/CD161+/CD154+/CD27−. Total peanut reactive cells are defined as CRTH2−/CD161+/CD154+/CD27−. In various embodiments, administration of TIMP-PPE in a subject maintains or decreases peanut-specific Th2a+ cells by about 1%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to placebo administration and/or one or more baseline measurements taken from the subject during treatment.

The proportion of activated peanut-specific T cells (activated peanut-specific T cells/total peanut specific T cells) after ex vivo stimulation with peanut proteins is determined by flow cytometry. Activated peanut-specific T cells are defined as CD154+/CD38+. Un-activated peanut-specific T cells are defined as CD154+. In various embodiments, administration of TIMP-PPE in a subject maintains or decreases activated peanut-specific T cells by about 1%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to placebo administration and/or one or more baseline measurements taken from the subject during treatment.

The frequency of T regulatory cell population (CD4+/CD25+/FoxP3+/Helios+/IL-10+) or (CD4+CD45RA^{low}CD4+CD137+CD25+CD127^{low}) is determined by flow cytometry. Multicolor flow analysis is performed to provide the proportion of peanut specific T regulatory cells (peanut specific T regulatory cells/peanut specific CD4+ effector memory cells). In various embodiments, administration of TIMP-PPE in a subject increases Tregs cells by about 1%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to placebo administration and/or one or more baseline measurements taken from the subject during treatment.

The ratio of cells' IL-5 to IFN-γ following in the PBMC culture supernatant is measured, e.g., as detected by Luminex 200.

The following indicators of immune tolerance status can be examined from the assay of basophils isolated from one or more blood samples collected from the subject and stimulated ex vivo with purified antigenic peanut proteins: Proportion of activated CD203+/CD63+ basophils after of ex vivo stimulation with purified antigenic peanut proteins using a basophil activation test (BAT) (Santos and Lack 2016 Clin Transl Allergy. 6: 10) and effective concentration at 50% of maximal basophil activation (EC50) after ex vivo stimulation with purified antigenic peanut protein measured using a basophil activation test where activated basophils are CD203+/CD63+/−. Analysis is performed to provide the effective concentration at 50% (EC50) of maximal basophil activation.

The following indicators of immune tolerance status can be examined from the assay of serum isolated from one or more blood samples obtained from the subject: Ratio of peanut specific IgE to IgG as measured by ImmunoCap assay. In various embodiments, administration of TIMP-PPE in a subject decreases the peanut specific IgE:IgG ratio by about 1%-100% (e.g. about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to placebo administration and/or one or more baseline measurements taken from the subject during treatment.

The change in peanut specific IgE as measured by ImmunoCap assay. In various embodiments, administration of TIMP-PPE in a subject maintains or decreases the levels of peanut specific IgE by about 1%-100% (e.g. about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values relative to placebo administration and/or one or more baseline measurements taken from the subject during treatment.

The following indicators of immune tolerance status can be examined from the assay of basophils isolated from one or more blood samples collected from the subject and stimulated ex vivo with purified antigenic peanut proteins: Proportion of activated CD203+/CD63+ basophils after of ex vivo stimulation with purified antigenic peanut proteins using a basophil activation test (BAT) (Santos and Lack 2016 Clin Transl Allergy. 6: 10) and effective concentration at 50% of maximal basophil activation (EC50) after ex vivo stimulation with purified antigenic peanut protein measured using a basophil activation test where activated basophils are CD203+/CD63+/−. Analysis is performed to provide the effective concentration at 50% (EC50) of maximal basophil activation. In various embodiments, administration of TIMP-PPE decreases basophil activation. In various embodiments, administration of TIMP-PPE in a subject increases the EC50 of maximal basophil activation by about 1%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-10,000-fold (e.g., about 2, 5, 10, 15, 20, 25, 30, 35, 40, 45, 50, 55, 60, 65, 70, 75, 80, 85, 90, 95, 100, 1000, or 10,000-fold inclusive of all values and ranges between these values) relative to placebo administration and/or one or more baseline measurements taken from the subject during treatment.

In certain embodiments, the efficacy of TIMP-PPE at relieving one or more symptoms of Peanut Allergy, increasing tolerance to peanut proteins, and/or reducing the duration and severity of an allergic immune response to peanut proteins is determined by a double-blind placebo-controlled food challenge (DBPCFC), change in the cumulative tolerated dose of peanut protein administered during a DBPCFC, and/or a skin prick test (SPT). Procedures for performing a DBPCFC and SPT have been previously described previously (Sampson et al., J Allergy Clin Immunol. 2012; 130(6):1260-1274; Heinzerling et al. The skin prick test—European standards. Clin Transl Allergy. 2013; 3(1):3).

The efficacy of TIMP-PPE at relieving one or more symptoms of Peanut Allergy and/or reducing the duration and severity of an allergic immune response to peanut proteins is determined from the assay of one or more biological samples from the subject. Biological samples include whole-blood, peripheral blood, peripheral blood mononuclear cells (PBMCs), serum, plasma, urine, cerebrospinal fluid (CSF), stool, a tissue biopsy, and/or a bone-marrow biopsy. In various embodiments, the assay of the biological sample(s) includes analyzing levels of, and or presence or absence of, cell-surface proteins, extracellular proteins, intracellular proteins, nucleic acids, metabolites, and/or combinations thereof.

The immune tolerance signature of a subject is generated using one or more of the following parameters assayed from one or more biological samples obtained from the subject and stimulated in vivo and/or ex vivo:
a. proportion of effector T cells in the total T cell population,
b. proportion of Treg cells in the total T cell population,
c. proportion of effector B cells in the total B cell population,
d. levels of specific IgG, IgA, IgM, and/or IgE,
e. levels of inflammatory cytokines and chemokines,
f. levels of anti-inflammatory cytokines and chemokines,
g. levels of inflammatory metabolites, and
h. levels of anti-inflammatory metabolites.

The immune tolerance signature is indicative of maintenance of immune tolerance if 1, 2, 3, 4, 5, 6, 7, or 8 parameters listed in (a)-(h) above indicate maintenance of immune tolerance. In various embodiments, the immune tolerance signature is indicative of maintenance of immune tolerance if at least 2/8 parameters listed in (a)-(h) indicate maintenance of immune tolerance. In various embodiments, the subject is determined to not require treatment with TIMPs if 1, 2, 3, 4, 5, 6, 7, or 8 parameters listed in (a)-(h) above indicate maintenance of immune tolerance. In various embodiments, the subject is determined to not require treatment with TIMPs if at least 3/8 parameters listed in (a)-(h) above indicate maintenance of immune tolerance.

The immune tolerance signature of a subject generated using one or more parameters described herein indicates weakening and/or absence of immune tolerance prior to or after treatment with TIMP-PPE, if:
a. the proportion of effector T cells in the total T cell population is between 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), and/or
b. the proportion of Treg cells in the total T cell population is between 1-3%, and/or
c. the proportion of effector B cells in the total B cell population is between 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), and/or
d. the levels of IgG, IgA, IgM, and/or IgE are increased by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment, and/or
e. levels of inflammatory cytokines/chemokines are increased by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment, and/or
f. levels of anti-inflammatory cytokines and chemokines are decreased by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment, and/or
g. levels of inflammatory metabolites are increased by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment, and/or
h. levels of anti-inflammatory metabolites are decreased by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment.

It is hypothesized that the proportion of Th2a+ cells at the pre-dose Day 1 timepoint are expected to be >15% in peanut allergic subjects. Treatment with TIMP-PPE is expected to reduce the proportion of Th2a+ cells to <15% 14 days after the second dose indicative of induction of immunological tolerance. Increase in the proportion of Th2a+ cells to >15% at any of the subsequent timepoints (e.g., Days 90, 180, 270, and 360 post-dose) would be indicative of weakening of immunological tolerance and warrant re-administration of TIMP-PPE for restoration of immunological tolerance. In combination, results from the above analyses can be used to determine an immune tolerance signature and whether the subject has maintained immunological tolerance or not. If such analyses indicate weakening and/or loss of immunological tolerance, TIMP-PPE may be re-administered to the subject to restore immunological tolerance.

### Pharmaceutical Formulations

Pharmaceutical compositions of the present disclosure containing the TIMP-PPE described herein as an active ingredient may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carbox-yvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene gly-col, Vaseline, paraffin, stearyl alcohol, stearic acid, human serum albumin (HSA), mannitol, sor-bitol, lactose, a pharmaceutically acceptable surfactant and the like. Additives used are chosen from, but not limited to, the above or combinations thereof, as appropriate, depending on the dosage form of the present disclosure.

Formulation of the pharmaceutical composition will vary according to the route of administration selected (e.g., solution, emulsion). An appropriate composition comprising the therapeutic to be administered can be prepared in a physiologically acceptable vehicle or carrier. For solutions or emulsions, suitable carriers include, for example, aqueous or alcohol-ic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Therapeutic formulations of the inhibitors are prepared for storage by mixing the inhibitor having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl para-bens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN^{T}M, PLURONICS™ or polyethylene glycol (PEG).

The formulations to be used for in vivo administration must be sterile. This is readily accomplished by filtration through sterile filtration membranes.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The TIMP-PPE described herein can be lyophilized for storage and reconstituted in a suitable carrier prior to use.

Solid dosage forms for oral administration include capsules, tablets, pills, powders, and granules. In such solid dosage forms, the modified particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

### Kits

As an additional aspect, the disclosure includes kits which comprise one or more compounds or compositions packaged in a manner which facilitates their use to practice methods of the disclosure. In one embodiment, such a kit includes a compound or composition described herein (e.g., a composition comprising a TIMP alone or in combination with a second agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the inhibitor compositions.

Additional aspects and details of the disclosure will be apparent from the following examples, which are intended to be illustrative rather than limiting.

### Example 1—Phase I/II Trial of TIMP-PPE in Peanut Allergy

The present example describes a Phase 1b/2a randomized, double-blind, placebo-controlled two-part study to assess the safety, tolerability, pharmacodynamics, and efficacy of TIMP-PPE (CNP-201) in subjects ages 16-55 with peanut allergy.

CNP-201 consists of PLGA nanoparticles encapsulating purified peanut extract. CNP-201 particles have an average diameter of 400-800 nm and a negative zeta potential of between −32 mV and −50 mV. CNP-201 particles are supplied as a lyophilized formulation. CNP-201 particles are reconstituted in sterile water for injection and diluted in sterile saline (0.9% sodium chloride) prior to administration.

The study includes Part A and Part B.

PART A: Part A is a randomized, double-blind, placebo-controlled study of the safety of tolerability of ascending dose levels of CNP-201. Part A will enroll 3 cohorts to receive CNP-201 or Placebo at multiple ascending dose levels. Part B follows as a randomized, double-blind, placebo controlled repeat dose study using the safe and tolerable dose level of CNP-201 determined from Part A.

Subjects who meet all inclusion and no exclusion criteria after initial screening assessments undergo a Skin Prick Test (SPT) followed by a baseline Double-Blind, Placebo-Controlled Food Challenge (DBPCFC) (peanut and placebo (oat) challenge, administered on two separate days) to confirm an allergy to peanut. The SPT and DBPCFC is conducted with a study physician or staff trained to manage clinical emergencies present on site and with immediate access to emergency medications and equipment and within close proximity to hospital emergency departments for rapid delivery of urgent care if needed. Subjects who continue to meet all inclusion and no exclusion criteria after completing both days of the DBPCFC are eligible to be enrolled into the study.

All subjects who continue to meet all I/E criteria following the DBPCFC receive subcutaneous injections of omalizumab (XOLAIR®). The dose of omalizumab (XOLAIR®) follows the product label specified in the protocol and is determined by the subject's weight and serum IgE at the initial screening. Subjects are dosed either every 2 weeks or every four weeks according to the product label.

Subjects who continue to meet Inclusion/Exclusion criteria is randomized on Day 1 in a 2:1 ratio (Part A) or 1:1 ratio (Part B) to receive either CNP-201 or Placebo (0.9% Sodium Chloride USP) by intravenous (IV) infusion. Subjects are dosed with CNP-201 or Placebo on Day 1 and Day 8.

Subjects with peanut allergy eligible for enrollment in this study are defined based on the following inclusion criteria:
1. Men and non-pregnant women, ages 16 to 55 years inclusive.
2. Subjects with a Body Mass Index (BMI)≥18 and ≤32 and weight>30 kg and ≤150 kg at Screen.
3. Subjects with serum IgE≥30 IU/mL and ≤1500 IU/mL at Screen.
4. Subjects with physician-diagnosed peanut allergy or documented history of peanut allergy.
5. Subjects with a documented history of non-severe anaphylaxis (Grade≤3) to peanuts, including mild wheezing or dyspnea without hypoxia.
6. Subjects with peanut specific IgE>5 kU/L as measured by ImmunoCAP at Screen.
7. Subjects who are self-reported to be on a peanut free diet with no suspected peanut exposure, including any peanut food challenge, for at least 14 days prior to Screen and agreement to continue restriction to peanut exposure during the study with the exception of the study DBPCFCs.
8. Subjects with a positive skin prick test (SPT) to peanut with a change in wheal diameter>3 mm as compared to a negative control (50% glycerin) at Screen.
9. Subjects who are willing and able to provide Institutional Review Board (IRB) approved written informed consent.
10. Subjects with a positive peanut DBPCFC at Screen (baseline DBPCFC) with an eliciting dose of ≥10 mg and ≤300 mg of peanut protein.
11. Subjects with 15% peanut specific Th2a+ T cells (peanut specific Th2a+ cells/total peanut specific T cells) following ex vivo stimulation of PBMCs at Screen.

Subjects who meet all inclusion and no exclusion criteria after completing both screening visits including both sets of the baseline DBPCFC (peanut and placebo challenge, administered on two separate days) are enrolled into one of 3 dose escalation cohorts. Subjects are randomized in a 2:1 ratio to receive either CNP-201 or Placebo (0.9% sodium chloride injection) as a 200 mL intravenous infusion on Day 1 and Day 8.

The dose levels for the 3 cohorts are as follows: Cohort 1:250 mg, Cohort 2: 450 mg, Cohort 3: 650 mg. Dosing of subjects within a dose cohort are separated by at least 48 hours.

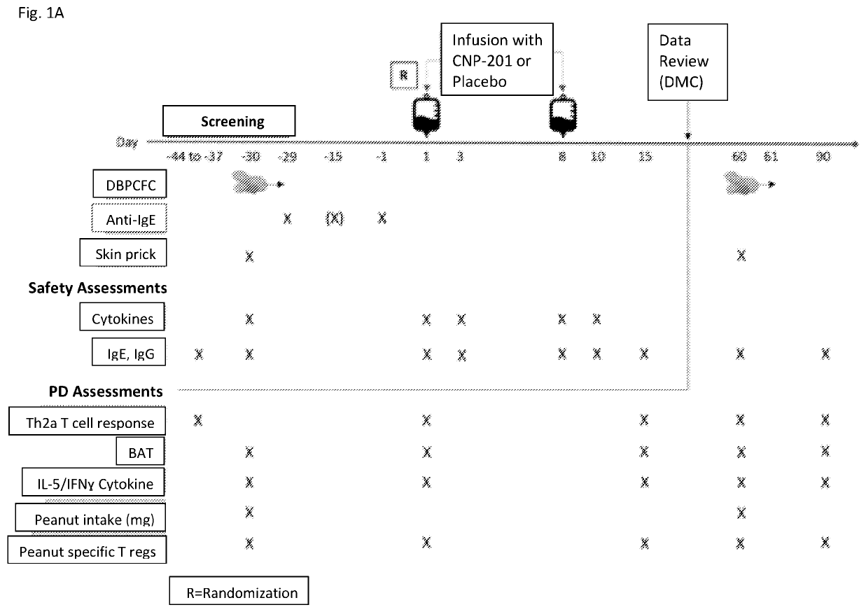

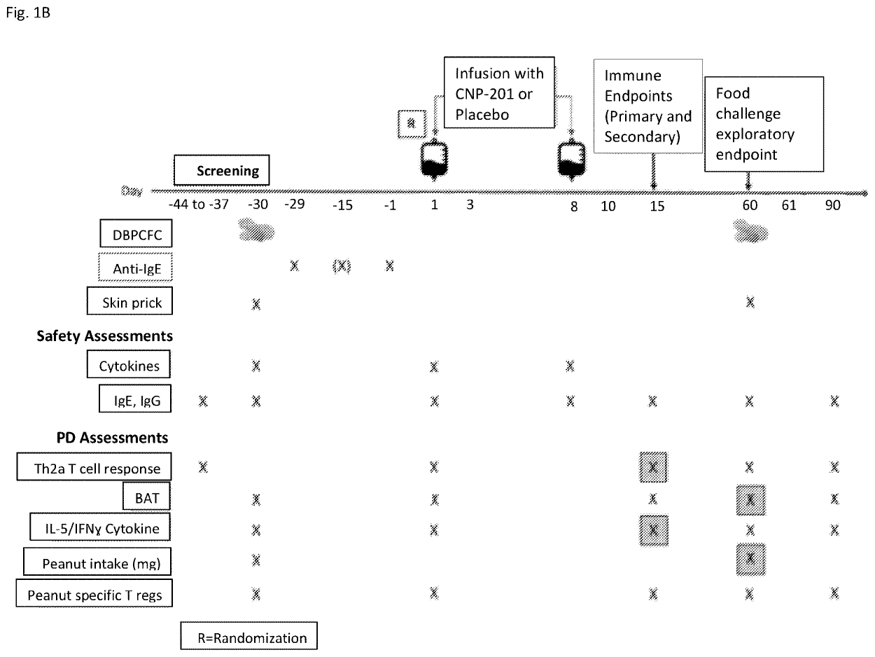

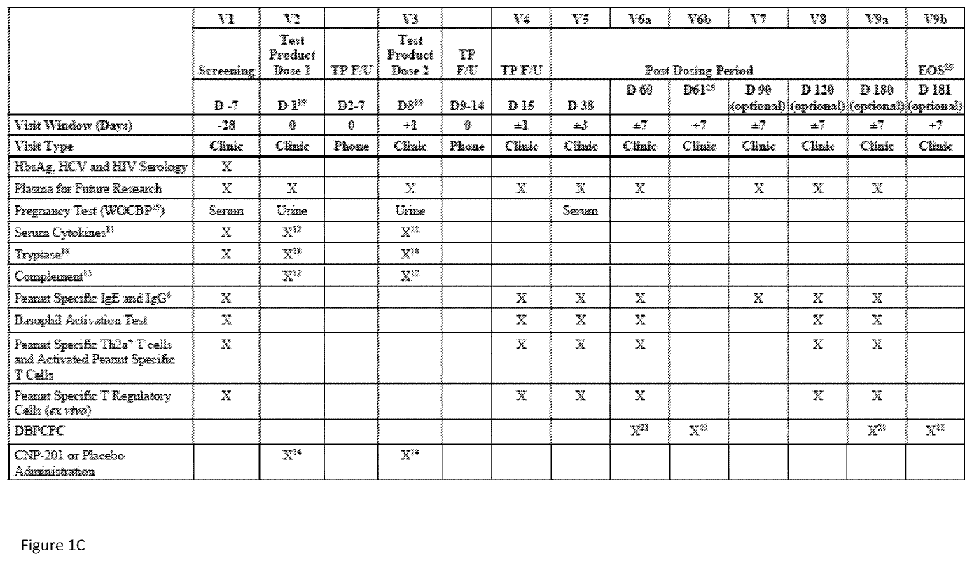

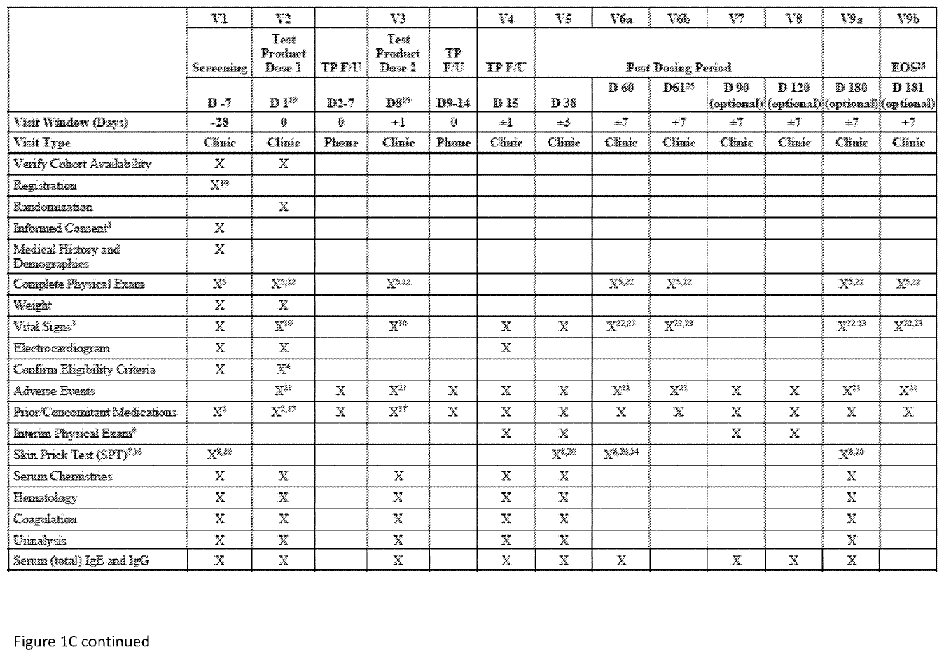

Subjects who meet all inclusion and no exclusion criteria after initial screening assessments will undergo a Skin Prick Test (SPT) followed by a baseline Double-Blind, Placebo-Controlled Food Challenge (DBPCFC) (peanut and placebo (oat) challenge, administered on two separate days) to confirm an allergy to peanut. If a reaction occurs and is treated, the peanut and placebo challenges are conducted at least 48 hours apart. Schedule of procedures for this study are depicted in FIG. 1C.

After completion of the second set of the DBPCFC and 2-hour observation period, subjects who continue to meet all inclusion and no exclusion criteria receive the first subcutaneous injection of omalizumab. The dose of omalizumab (XOLAIR®) follows the product label and is determined by the subject's weight and serum IgE at Screen as described in Table 2. Subjects are dosed either every 2 weeks (Days −29, −15 and −1) or every 4 weeks (Days −29 and −1) according to the product label.

TABLE 2
Omalizumab Dose Determination

| Screening | | Body Weight (kg) | | | | | | | |
|---|---|---|---|---|---|---|---|---|---|
| Serum IgE | Dosing | >30-40 | >40-50 | >50-60 | >60-70 | >70-80 | >80-90 | >90-125 | >125-150 |
| (IU/mL) | Freq. | Dose (mg) | | | | | | | |
| ≥30-100 | Every 4 | 75 | 150 | 150 | 150 | 150 | 150 | 300 | 300 |
| >100-200 | weeks | 150 | 300 | 300 | 300 | 300 | 300 | 450 | 600 |
| >200-300 | | 225 | 300 | 300 | 450 | 450 | 450 | 600 | 375 |
| >300-400 | | 300 | 450 | 450 | 450 | 600 | 600 | 450 | 525 |
| >400-500 | | 450 | 450 | 600 | 600 | 375 | 375 | 525 | 600 |
| >500-600 | | 450 | 600 | 600 | 375 | 450 | 450 | 600 | |
| >600-700 | | 450 | 600 | 375 | 450 | 450 | 525 | | |
| >700-800 | Every 2 | 300 | 375 | 450 | 450 | 525 | 600 | | |
| >800-900 | weeks | 300 | 375 | 450 | 525 | 600 | | | |
| >900-1000 | | 375 | 450 | 525 | 600 | | | | |
| >1000-1100 | | 375 | 450 | 600 | | | | | |
| >1100-1200 | | 450 | 525 | 600 | | | | | |
| >1200-1300 | | 450 | 525 | | | | | | |
| >1300-1500 | | 525 | 600 | | | | | | |

Any subject who experiences a severe hypersensitivity reaction to omalizumab is provided with appropriate treatment and discontinued from the study, then replaced with an additional subject at the same dose level.

Subjects return to the clinic on Day 1 for final assessment of eligibility and collection of laboratory samples. Subjects who continue to meet all inclusion and no exclusion criteria are randomized into the dose Cohort open at that time. Subjects are dosed with CNP-201 or Placebo on Day 1 and on Day 8. CNP-201 or Placebo is administered by intravenous infusion over approximately 3-4 hours using a graduated rate of infusion. Subjects undergo medical observation in the clinic for acute adverse events (AEs) including infusion reactions (IR) for 4 hours following the infusion. Antihistamines/epinephrine is immediately available to treat allergic reactions, should they occur.

Subjects return for an office visit 2 days after each infusion (Day 3 and 10) for collection of safety labs, review of medications, and assessment of AEs and are followed daily through telephone visits between infusions (Days 33-36) to assess and document any AEs and medication changes. In the post-dosing period, subjects return to the clinic on Day 15 for collection of safety labs, PD measurements, and assessment of AEs and medication changes.

After all subjects in a dose Cohort complete the Day 15 office visit (7 days post-second dose), the Data Monitoring Committee (DMC) are convened to review all available safety data and determine whether it is acceptable to proceed to the next ascending dose Cohort, if an expansion of the Cohort is warranted (minimum of three additional subjects randomized 2:1 to receive CNP-201 or Placebo (0.9% Sodium Chloride USP)), or if any other clinical recommendations should made.

The subject returns to the clinic on Day 60 for collection of immune safety labs, PD measurements, and a second SPT followed by a DBPCFC (to be completed on Day 61). The subject will return to the clinic for the end of study visit on Day 90 for collection of safety labs, PD measurements, and final assessment of AEs and medication changes. Once all subjects have completed the Day 15 visit, recommendations regarding continuation into Part B with a safe and tolerable dose of CNP-201 identified in Part A are made.

PART B: Subjects in Part B are randomized in a 1:1 ratio to receive a safe and tolerable level of CNP-201 identified in Part A or Placebo (0.9% Sodium Chloride USP). Subjects who meet all inclusion and no exclusion criteria after initial screening assessments undergo a Skin Prick Test (SPT) followed by a baseline Double-Blind, Placebo Controlled Food Challenge (DBPCFC) (peanut and placebo (oat) challenge, administered on two separate days) to confirm an allergy to peanut. If a reaction occurs and is treated, the peanut and placebo challenges are conducted at least 48 hours apart. After completion of the second set of the DBPCFC and 2-hour observation period, subjects who continue to meet all inclusion and no exclusion criteria will receive the first subcutaneous injection of omalizumab.

The dose of omalizumab (XOLAIR®) follows the product label specified in the protocol and is determined by the subject's weight and serum IgE at Screen and described in Table 2. Subjects are dosed either every 2 weeks or every 4 weeks according to the product label. Any subject who experiences a severe hypersensitivity reaction to omalizumab is provided with appropriate treatment and discontinued from this study. These subjects are replaced in Part B.

Subjects return to the clinic on Day 1 for final assessment of eligibility and collection of laboratory samples. Subjects who continue to meet all inclusion and no exclusion criteria are randomized to receive either CNP-201 or Placebo.

Subjects will receive CNP-201 or Placebo on Day 1 and Day 8. Investigational Product is administered by IV infusion over approximately 3-4 hours using a graduated rate of infusion. Subjects undergo medical observation in the clinic for acute AEs for 4 hours following the infusion. Antihistamines/epinephrine are immediately available to treat allergic reactions, should they occur. Subjects are followed through daily telephone visits between each infusion (Day 2-7 and 9-14) to assess and document any AEs and medication changes.

In the post-dosing period, subjects return to the clinic on Day 15 for collection of safety labs, PD measurements, and assessment of AEs and medication changes. Subjects also return to the clinic on Day 60 for immune safety labs, PD measurements, and a second SPT followed by a DBPCFC (to be completed on Day 61). Subjects return to the clinic for the end of study visit on Day 90 for collection of safety labs, PD measurements, and final assessment of AEs and medication changes.

Emerging safety and tolerability data is monitored in Part B. The Medical Monitor is notified of any serious adverse event (SAE) and any>Grade 2 adverse event (CTCAE v.5.0) within 24 hours of becoming aware of such an event. The Medical Monitor may then convene an ad-hoc DMC meeting to evaluate safety and tolerability data to determine if it remains acceptable to continue dosing and make recommendations including, but not limited to, continuation of dosing and stopping or pausing dosing in the subject. The DMC may at any point during the study recommend stopping or pausing the study if, in their opinion, continuation of dosing poses a safety risk to subjects.

In both Part A and Part B of the study, subjects receive CNP-201 via intravenous infusion lasting approximately 3-4 hours according to the following graduated infusion rate: 20 mL/hr for the first 15 minutes, 40 mL/hr for the next 15 minutes, 80 mL/hr for the remainder of the infusion.

Study Duration: 2 doses, 7 days apart (Part A and B). The total duration of the study for an individual subject is ~134 days; 14 days for Screening, 30 days for omalizumab dosing, 60 days for IP dosing, and a 30 Day follow up period.

Primary Endpoints (Part A and Part B) include: Frequency of Adverse Events (AEs) and Serious Adverse Events (SAEs), MedDRA 23.0 (CTCAE v.5.0); Laboratory Safety Assessments (hematology, serum chemistry, coagulation panel, urinalysis); Physical Examinations including vital signs (blood pressure, heart rate, temperature); 12-lead electrocardiogram (ECG) 12-lead electrocardiogram; Serum Cytokines (TNF-α, IL-2, IL-6, IL-8, IL-1β, MCP-1, MIP-1β, MIP-1α, IFN-γ, IL-12p70); Change in the proportion of peanut specific Th2a⁺ T cells (peanut specific Th2a⁺ cells/total peanut specific T cells) following ex vivo stimulation of PBMCs between placebo and CNP-201 at baseline (Day 1 pre-dose) and at Day 15; and Change in the proportion of activated peanut specific T cells to total peanut specific T cells following ex vivo stimulation of PBMCs between placebo and CNP-201 at baseline (Day 1 pre-dose) and at Day 15.

Secondary Endpoints (Part A and Part B) include: Change in the ratio of IL-5 to IFN-γ following ex vivo stimulation of PBMCs between placebo and CNP-201 at baseline (Day 1 pre-dose) and at Day 15.

Exploratory Endpoints (Part A and Part B) include: Change in the proportion of peanut-specific T regulatory cells (peanut-specific T regulatory cells/peanut specific CD4+ effector memory cells) following ex vivo stimulation of PBMCs between placebo and CNP-201 at baseline (Day 1 pre-dose) and at Day 15; Change in the effective concentration at 50% of maximal basophil activation (EC₅₀) as measured by a Basophil Activation Test (CD203c+/CD63+/− basophil activation) between placebo and CNP-201 at baseline (Day −30 pre-DBPCFC) and at Day 60; Change in the ratio of peanut specific IgE to IgG as measured by ImmunoCap assay between placebo and CNP-201 at baseline (Day −30 pre-DBPCFC) and at Day 60; and Change in the cumulative tolerated dose (CTD) of peanut protein (mg) administered during a DBPCFC between placebo and CNP-201 at baseline (Day [−30]-[−29]) and at Day 60-61. Cell markers used for assessment of exploratory endpoints are described in Table 3 below:

TABLE 3
Cell Markers Used for Assessment of Exploratory Endpoints

| Cell Population | Markers |
|---|---|
| Effector memory cells | CD45RA^{low}, CD4+ |
| Peanut specific T cells | EM [CD45RA^{low}, CD4+], CD154+, CD137+ |
| Peanut specific Th2a+ cells | EM [CD45RA^{low}, CD4+], CD154+, CD137+, CXCR5−, CRTh2+/−, CD27−, |
| | CD200R+, CD161+ |
| Activated peanut specific T | EM [CD45RA^{low}, CD4+], CD154+, CD137+, CD38+ |
| cells | |
| Peanut specific CD4+ effector | EM [CD45RA^{low}, CD4+], CD137+ |
| memory cells | |
| Peanut specific T regulatory | EM CD4+ [CD45RA^{low}, CD4+, CD137+], CD25⁺, CD127^{low} |
| cells | |
| Basophils | CD45+, CD3−, SSCA^{low}, CRTh2+, HLADR−, CD123+ |
| Activated basophils | CD45+, CD3−, SSCA^{low}, CRTh2+, HLADR−, CD123+, CD203c+/CD63+/− |

### Example 1A—Phase I/II Trial of TIMP-PPE in Peanut Allergy

Provided is an alternative protocol for carrying out a Phase 1b/2a randomized, double-blind, placebo-controlled two-part study to assess the safety, tolerability, pharmacodynamics, and efficacy of TIMP-PPE (CNP-201) in subjects ages 16-35 with peanut allergy.

CNP-201 consists of PLGA nanoparticles encapsulating purified peanut extract having an average diameter of 400-800 nm and a negative zeta potential of between −30 mV and −60 mV. CNP-201 particles are supplied as a lyophilized formulation. CNP-201 particles are reconstituted in sterile water for injection and diluted in sterile saline (0.9% sodium chloride) prior to administration.

The study includes Part A and Part B. PART A: Part A is a randomized, double-blind, placebo-controlled study of the safety of tolerability of ascending dose levels of CNP-201. Part A will enroll 3 cohorts to receive CNP-201 or Placebo at multiple ascending dose levels. Part B follows as a randomized, double-blind, placebo controlled repeat dose study using the safe and tolerable dose level of CNP-201 determined from Part A.

Subjects who meet all inclusion (exception for IgG and IgE results which may not be available at visit 2) and no exclusion criteria after initial screening assessments undergo a Skin Prick Test (SPT) followed by a baseline Double-Blind, Placebo-Controlled Food Challenge (DBPCFC) (peanut and placebo (oat) challenge, administered on two separate days) to confirm an allergy to peanut. The SPT and DBPCFC is conducted with a study physician or staff trained to manage clinical emergencies present on site and with immediate access to emergency medications and equipment and within close proximity to hospital emergency departments for rapid delivery of urgent care if needed. Subjects who continue to meet all inclusion and no exclusion criteria after completing both days of the DBPCFC are eligible to be enrolled into the study.

All subjects who continue to meet all I/E criteria following the DBPCFC receive subcutaneous injections of omalizumab (XOLAIR). The dose of omalizumab (XOLAIR®) follows the product label specified in the protocol and is determined by the subject's weight and serum IgE at the initial screening. Subjects are dosed either every 2 weeks or every four weeks according to the product label.

Subjects who continue to meet all inclusion and no exclusion criteria are randomized on Day 1 in a 2:1 ratio (Part A) or 1:1 ratio (Part B) to receive either CNP-201 or Placebo (0.9% Sodium Chloride USP) by intravenous (IV) infusion. Subjects are administered CNP-201 or Placebo on Day 1 and Day 8. Subjects remain in the clinic on Day 1 and Day 8 from the time of admission (prior to administration of CNP-201 or Placebo) through the final procedure conducted 4 hours post-dose that same day unless an infusion reaction, anaphylaxis, or other adverse event requires an extended duration of monitoring. Subjects are discharged if safety parameters are acceptable to the investigator.

Subjects with peanut allergy eligible for enrollment in this study are defined based on following inclusion criteria:
1. Men and non-pregnant women, ages 16 to 35 years inclusive.
2. Subjects with a Body Mass Index (BMI)≥18 and ≤32 and weight>30 kg and ≤150 kg at Screening. Subjects who fall outside of this range may be included at the discretion of the investigator.
3. Subjects with serum IgE≥30 IU/mL and ≤1500 IU/mL at Screening. Subjects who fall outside of this range may be included at the discretion of the investigator.
4. Subjects with physician-diagnosed peanut allergy or documented history of peanut allergy.
5. Subjects with a documented history of non-severe anaphylaxis (Grade≤3) to peanuts, including mild wheezing or dyspnea without hypoxia.
6. Subjects with peanut specific IgE>2 kU/L as measured by ImmunoCAP at Screening and/or a positive skin prick test (SPT) to peanut with a change in wheal diameter>3 mm as compared to a negative control (50% glycerin) at Screening.
7. Subjects who are self-reported to be on a peanut free diet with no suspected peanut exposure, including any peanut food challenge, for at least 14 days prior to Screening and agreement to continue restriction to peanut exposure during the study with the exception of the study DBPCFCs.
8. Female subjects and male subjects and their female spouse/partners who are willing to practice a highly effective method of contraception that may include, but is not limited to, abstinence, sex only with persons of the same sex, monogamous relationship with vasectomized partner, vasectomy, hysterectomy, bilateral tubal ligation, licensed hormonal methods, intrauterine device (IUD), or use of spermicide combined with a barrier method (e.g., condom, diaphragm) starting at Screening and continuing throughout the entire study to Day 90 (EOS/ET).
9. Female subjects who agree to not breastfeed starting at initial Screening and throughout the entire study to Day 90 (EOS/ET).
10. Female subjects who agree to not donate ova starting at initial Screening and throughout the entire study to Day 90 (EOS/ET).
11. Subjects who are willing and able to provide Institutional Review Board (IRB) approved written informed consent.
12. Subjects who are willing to perform and comply with all study procedures including attending study visits as scheduled and completing two DBPCFCs.
13. Male subjects who agree to not donate sperm starting at Screening and throughout the entire study to Day 90 (EOS/ET).

Subjects must have a positive peanut DBPCFC at Screening with an eliciting dose of ≥10 mg and ≤300 mg of peanut protein in order to be included in statistical analysis for exploratory endpoints. Subjects who tolerate>444 mg of peanut (cumulative tolerated dose) will be followed for safety and evaluated separately.

Subjects who meet all inclusion and no exclusion criteria after completing both screening visits including both sets of the baseline DBPCFC (peanut and placebo challenge, administered on two separate days) are enrolled into one of 3 dose escalation cohorts. Subjects are randomized in a 2:1 ratio to receive either CNP-201 or Placebo (0.9% sodium chloride injection) as a 200 mL intravenous infusion on Day 1 and Day 8. The dose levels for the 3 cohorts are as follows: Cohort 1: 250 mg, Cohort 2: 450 mg, Cohort 3: 650 mg. Dosing of subjects within a dose cohort are separated by at least 48 hours.

Subjects who meet all inclusion and no exclusion criteria after initial screening assessments will undergo a Skin Prick Test (SPT) followed by a baseline Double-Blind, Placebo-Controlled Food Challenge (DBPCFC) (peanut and placebo (oat) challenge, administered on two separate days) to confirm an allergy to peanut. If a reaction occurs and is treated, the peanut and placebo challenges are conducted at least 48 hours apart.

After completion of the second challenge of the DBPCFC and 2 hour observation period, subjects who continue to meet all inclusion and no exclusion criteria will proceed to the first subcutaneous injection of XOLAIR®.

The dose of XOLAIR® and dosing frequency (every 2 weeks or every 4 weeks) is determined by the subject's serum IgE at Screening and weight measured at the XOLAIR® Dose 1 as described in Table 2.

Subjects return to the clinic on Day 1 for final assessment of eligibility and collection of laboratory samples. Subjects who continue to meet all inclusion and no exclusion criteria are randomized into the dose Cohort open at that time. Subjects are administered CNP-201 or Placebo on Day 1 and on Day 8. CNP-201 or Placebo administered by intravenous infusion over approximately 3-4 hours using a graduated rate of infusion. Subjects undergo medical observation in the clinic for acute adverse events (AEs) including infusion reactions (IR) for 4 hours following the infusion. Antihistamines/epinephrine will be immediately available to treat allergic reactions, should they occur.

If two of the same Grade 3 adverse events (AE) or one Grade 4 (CTCAE v.5.0 or CoFAR V.1 for allergy related AEs) occur that are considered to be likely related to CNP-201, dosing is paused, and the DMC will convene to review all available safety data acquired to date. Grade 4 or Grade 5 AEs considered to be serious AEs (SAE) is reported to the Agency. After review of all available safety data, the DMC will make recommendations including, but not limited to, stopping of dosing, de-escalation of dosing, continuation of dosing, expanding the current Cohort, adding a Cohort in Part A, or determination of dose limiting toxicity. The DMC may also convene ad-hoc to address emerging safety concerns during dosing of any subject in Part A based on ongoing daily surveillance of safety data by the Sponsor medical monitor. The DMC will evaluate the available safety data including, but not limited to, AEs, physical examinations, vital signs, 12 lead ECG, and available laboratory results.

Subjects return for an office visit 2 days after each infusion (Day 3 and 10) for collection of safety labs, review of medications, and assessment of AEs and are followed daily through telephone visits following each infusion (Days 4-7 and Days 11-14) to assess and document any AEs and medication changes. In the Post-Dosing period, subjects return to the clinic 7 days after administration of Dose 2 of CNP-201 or Placebo for collection of safety labs, PD measurements, and assessment of AEs and medication changes.

After all subjects in a dose Cohort have completed the Day 15 office visit (7 days post-second dose), the DMC is convened to review all available safety data and determine whether it is acceptable to proceed to the next ascending dose Cohort, if an expansion of the Cohort is warranted (minimum of three additional subjects randomized 2:1 to receive CNP-201 or Placebo), or if any other clinical recommendations should made (e.g., expansion to Part B).

The subject returns to the clinic on Day 60 for collection of immune safety labs, PD measurements, and a second SPT followed by the Post-Dosing DBPCFC, and returns to the clinic for the end of study visit on Day 90 for collection of safety labs, PD measurements, and final assessment of AEs and medication changes. Emerging safety and tolerability data is to be monitored continually. The Medical Monitor is notified of any serious adverse event (SAE) and any >Grade 2 adverse event likely related to test product (CTCAE v.5.0 or CoFAR V.1) within 24 hours of becoming aware of such an event. The Medical Monitor may then convene an ad-hoc DMC meeting to evaluate safety and tolerability data to determine if it remains acceptable to continue dosing and make recommendations including, but not limited to, continuation of dosing and stopping or pausing dosing in the subject. The DMC may at any point during the study recommend stopping or pausing the study if, in their opinion, continuation of dosing poses an unacceptable safety risk to subjects. Once all subjects have completed the Day 15 visit, recommendations regarding continuation into Part B with a safe and tolerable dose of CNP-201 identified in Part A are made.

Part B: Subjects in Part B are randomized in a 1:1 ratio to receive a safe and tolerable level of CNP-201 identified in Part A or Placebo. Subjects in Part A and Part B undergo the same assessments with the only difference between the two parts being the dose escalation in Part A.

Subjects who meet all inclusion and no exclusion criteria after initial screening assessments undergo a Skin Prick Test (SPT) followed by a baseline Double-Blind, Placebo-Controlled Food Challenge (DBPCFC) (peanut and placebo (oat) challenge, administered on two separate days) to confirm an allergy to peanut. If a reaction occurs and is treated, the peanut and placebo challenges are conducted at least 48 hours apart.

After completion of the second challenge of the DBPCFC and 2 hour observation period, subjects who continue to meet all inclusion and no exclusion criteria proceed to the first subcutaneous injection of XOLAIR®. The dose of XOLAIR® and dosing frequency (every 2 weeks or every 4 weeks) is determined by the subject's serum IgE at Screening and weight measured at the XOLAIR® Dose 1 as described in Table 2.

Subjects return to the clinic on Day 1 for final assessment of eligibility and collection of laboratory samples. Subjects who continue to meet all inclusion and no exclusion criteria are randomized to receive either CNP-201 or Placebo.

Subjects are administered CNP-201 or Placebo on Day 1 and on Day 8. CNP-201 or Placebo are administered by intravenous infusion over approximately 3-4 hours using a graduated rate of infusion. Subjects undergo medical observation in the clinic for acute adverse events (AEs) including infusion reactions (IR) for 4 hours following the infusion. Antihistamines/epinephrine are immediately available to treat allergic reactions, should they occur.

Subjects return for an office visit 2 days after each infusion (Day 3 and 10) for collection of safety labs, review of medications, and assessment of AEs and are followed daily through telephone visits following each infusion (Days 4-7 and Days 11-14) to assess and document any AEs and medication changes. In the Post-Dosing period, subjects return to the clinic 7 days after administration of Dose 2 of CNP-201 or Placebo for collection of safety labs, PD measurements, and assessment of AEs and medication changes.

The subject returns to the clinic on Day 60 for collection of immune safety labs, PD measurements, and a second SPT followed by the Post-Dosing DBPCFC, and returns to the clinic for the end of study visit on Day 90 for collection of safety labs, PD measurements, and final assessment of AEs and medication changes. Emerging safety and tolerability data is to be monitored continually. The Medical Monitor is notified of any serious adverse event (SAE) and any >Grade 2 adverse event likely related to test product (CTCAE v.5.0 or CoFAR V.1) within 24 hours of becoming aware of such an event. The Medical Monitor may then convene an ad-hoc DMC meeting to evaluate safety and tolerability data to determine if it remains acceptable to continue dosing and make recommendations including, but not limited to, continuation of dosing and stopping or pausing dosing in the subject. The DMC may at any point during the study recommend stopping or pausing the study if, in their opinion, continuation of dosing poses an unacceptable safety risk to subjects.

In both Part A and Part B of the study, subjects receive CNP-201 via intravenous infusion lasting approximately 3-4 hours according to the following graduated infusion rate: 20 mL/hr for the first 15 minutes, 40 mL/hr for the next 15 minutes, 80 mL/hr for the remainder of the infusion.

In both Part A and Part B, subjects randomized to Placebo receive 0.9% Sodium Chloride for injection (Normal Saline [NS]). Placebo is administered as a 200 mL intravenous infusion on Day 1 and Day 8 according to the following graduated infusion rate: 20 mL/hr for the first 15 minutes, 40 mL/hr for the next 15 minutes, 80 mL/hr for the remainder of the infusion.

Study duration: 2 doses, 7 days apart (Part A and B). The total duration of the study for an individual subject is ~134 days; 14 days for Screening, ~30 days for XOLAIR® dosing, 60 days for Test Product dosing, and ~30 days for Post-Dosing Follow-Up.

Primary Endpoints (Part A and Part B) include: Frequency of Adverse Events (AEs) and Serious Adverse Events (SAEs), MedDRA 23.0 (CTCAE v.5.0 or CoFAR V.1 for allergy related AEs); Laboratory Safety Assessments (hematology, serum chemistry, coagulation panel, and urinalysis); Physical Examinations including vital signs (blood pressure, heart rate, and temperature); 12-lead electrocardiogram (ECG); Serum Cytokines (TNF-α, IL-2, IL-6, IL-8, IL-1β, MCP-1, MIP-1β, MIP-1α, IFN-γ, and IL-12p70).

Exploratory Endpoints (Part A and Part B) include: Change in the proportion of peanut specific Th2a+ T cells (peanut specific Th2a+ cells/total peanut specific T cells) following ex vivo stimulation of PBMCs between placebo and CNP-201 at baseline (Day 1 pre-dose) and at Day 15; Change in the effective concentration at 50% of maximal basophil activation (EC50) as measured by a Basophil Activation Test (CD203c+/CD63+/− basophil activation) between placebo and CNP-201 at baseline (Screening, Visit 2 pre-DBPCFC) and at Visit 11; Change in the peanut specific IgE as measured by ImmunoCap assay between placebo and CNP-201 at baseline (Screening, Visit 2 pre-DBPCFC) and at Visit 11; Change in the ratio of peanut specific IgE to IgG as measured by ImmunoCap assay between placebo and CNP-201 at baseline (Screening, Visit 2 pre-DBPCFC) and at Visit 11; Change in the cumulative tolerated dose (CTD) of peanut protein (mg) administered during a DBPCFC between placebo and CNP-201 at baseline (Screening DBPCFC, Visits 2 and 3) and Post-Dosing (Post-dosing DBPCFC, Visits 11 and 12); Change in the proportion of activated peanut specific T cells (activated peanut specific T cells/total peanut specific T cells) following ex vivo stimulation of PBMCs between placebo and CNP-201 at baseline (Day 1 pre-dose) and at Day 15.

### Example 2—Phase I/II Trial of TIMP-PPE without IgE Inhibitors in Peanut Allergy

The present example describes a Phase 1b/2a randomized, double-blind, placebo-controlled study to assess the safety, tolerability and pharmacodynamics of TIMP-PPE (CNP-201) without administration of IgE inhibitors, in subjects ages 16-55 with peanut allergy.

CNP-201 consists of PLGA nanoparticles encapsulating purified peanut extract having an average diameter of 400-800 nm and a negative zeta potential of between −30 mV and −60 mV. CNP-201 particles are supplied as a lyophilized formulation. CNP-201 particles are reconstituted in sterile water for injection and diluted in sterile saline (0.9% sodium chloride) prior to administration.

The study is a randomized, double-blind, placebo-controlled study of the safety and tolerability of ascending dose levels of CNP-201. The study will enroll 3 cohorts to receive CNP-201 or Placebo at multiple ascending dose levels.

Subjects who meet all inclusion and no exclusion criteria are enrolled into the study. Subjects who continue to meet Inclusion/Exclusion criteria are randomized on Day 1 in a 2:1 ratio to receive either CNP-201 or Placebo (0.9% Sodium Chloride USP) by intravenous (IV) infusion. Subjects are dosed with CNP-201 or Placebo on Day 1 and Day 8.

Subjects may receive pre-administration of an antihistamine (such as 10 mg IV cetirizine) and a corticosteroid (such as 125 mg IV methylprednisolone) 30 minutes prior to each infusion of CNP-201 or Placebo. Subjects will remain on their current SoC during the study, excluding a 12 hour washout period (for beta-agonists, theophylline, and cromolyn only) and a 7 day washout period for antihistamines prior to skin prick test (SPT). Subjects will re-commence their SoC regimen after SPT and remain on SoC through dosing/duration of the study.

Subjects with peanut allergy eligible for enrollment in this study are defined based on the following inclusion criteria:
1. Men and non-pregnant women, ages 16 to 55 years inclusive.
2. Subjects with physician-diagnosed peanut allergy or documented history of peanut allergy.
3. Subjects with weight≥31.25 kg at Screening. Subjects who fall outside of this range may be included at the discretion of the investigator.
4. Subjects with a documented history of non-severe anaphylaxis (Grade≤3) to peanuts, including mild wheezing or dyspnea without hypoxia.
5. Subjects with peanut specific IgE>5 kU/L as measured by ImmunoCAP at Screening.
6. Subjects who are self-reported to be on a peanut free diet with no suspected peanut exposure, including any peanut food challenge, for at least 14 days prior to Screening and agreement to continue restriction to peanut exposure during the study.
7. Subjects with a positive skin prick test (SPT) to peanut with a change in wheal diameter>3 mm as compared to a negative control (50% glycerin) at Screening.
8. Female subjects and male subjects and their female spouse/partners who are willing to practice a highly effective method of contraception that may include, but is not limited to, abstinence, sex only with persons of the same sex, monogamous relationship with vasectomized partner, vasectomy, hysterectomy, bilateral tubal ligation, licensed hormonal methods, intrauterine device (IUD), or use of spermicide combined with a barrier method (e.g., condom, diaphragm) starting at Screening and continuing throughout the entire study to Day 38 (EOS/ET).
9. Female subjects who agree to not breastfeed starting at initial Screening and throughout the entire study to Day 38 (EOS/ET).
10. Female subjects who agree to not donate ova starting at initial Screening and throughout the entire study to Day 38 (EOS/ET).
11. Subjects who are willing and able to provide Institutional Review Board (IRB) approved written informed consent.
12. Subjects who are willing to perform and comply with all study procedures.
13. Male subjects who agree to not donate sperm starting at Screening and throughout the entire study to Day 38 (EOS/ET).

Subjects who meet all inclusion and no exclusion criteria after completing the Screening visit are enrolled into one of 3 dose escalation cohorts. Subjects are randomized in a 2:1 ratio to receive either CNP-201 or Placebo (0.9% sodium chloride injection) as a 200 mL intravenous infusion on Day 1 and Day 8. The dose levels for the 3 cohorts are as follows: Cohort 1: 250 mg, Cohort 2: 450 mg, Cohort 3: 650 mg. Dosing of subjects within a dose cohort are separated by at least 48 hours.

Subjects who meet all inclusion and no exclusion criteria at Screening are enrolled into the study. Subjects will return to the clinic on Day 1 for final assessment of eligibility and collection of laboratory samples. Subjects who continue to meet all inclusion and no exclusion criteria are randomized into the dose Cohort open at that time. Subjects may receive pre-administration of an antihistamine (such as 10 mg IV cetirizine) and a corticosteroid (such as 125 mg IV methylprednisolone) 30 minutes prior to each infusion of CNP-201 or Placebo on Day 1 and on Day 8. CNP-201 or Placebo are administered by intravenous infusion over approximately 3-4 hours using a graduated rate of infusion. Subjects undergo medical observation in the clinic for acute adverse events (AEs) including infusion reactions (IR) for 4 hours following the infusion. Antihistamines/epinephrine, including intramuscular (IM) and intravenous (IV) epinephrine, are immediately available to treat allergic reactions, should they occur.

Subjects are followed daily through telephone visits following each infusion (Days 2-7 and Days 9-14) to assess and document any AEs and medication changes. In the Post-Dosing period, subjects return to the clinic 7 days after administration of Dose 2 of CNP-201 or Placebo (Day 15) for collection of safety labs, PD measurements, and assessment of AEs and medication changes.

After all subjects in a dose Cohort have completed the Day 15 office visit (7 days post-second dose), the DMC is convened to review all available safety data and determine whether it is acceptable to proceed to the next ascending dose Cohort.

The subject returns to the clinic for the end of study visit on Day 38 for collection of safety labs, PD measurements, and final assessment of AEs and medication changes and a second SPT.

Emerging safety and tolerability data is to be monitored continually. The Medical Monitor is notified of any serious adverse event (SAE) and any ≥Grade 2 adverse event likely related to test product (CTCAE v.5.0 or CoFAR V.3.0) within 24 hours of becoming aware of such an event. The Medical Monitor may convene an ad-hoc DMC meeting to evaluate safety and tolerability data to determine if it remains acceptable to continue dosing and make recommendations including, but not limited to, continuation of dosing and stopping or pausing dosing in the subject. The DMC may at any point during the study recommend stopping or pausing the study if, in their opinion, continuation of dosing poses an unacceptable safety risk to subjects.

Subjects receive CNP-201 via intravenous infusion lasting approximately 3-4 hours according to the following graduated infusion rate: 20 mL/hr for the first 15 minutes, 40 mL/hr for the next 15 minutes, 80 mL/hr for the remainder of the infusion.

Study duration: 2 doses, 7 days apart. The total duration of the study for an individual subject is ~45 days; 7 days for Screening; 8 days for Test Product dosing; and 30 days for post-dosing evaluation.

Primary endpoints include: Frequency of Adverse Events (AEs) and Serious Adverse Events (SAEs), MedDRA 23.0 (CTCAE v.5.0 or CoFAR V.3.0 for allergy related AEs); Laboratory Safety Assessments (hematology, serum chemistry, coagulation panel, and urinalysis); Physical Examinations including vital signs (blood pressure, heart rate, and temperature); 12-lead electrocardiogram (ECG); Serum Cytokines (TNF-α, IL-2, IL-6, IL-8, IL-1β, MCP-1, MIP-1β, MIP-1α, IFN-γ, and IL-12p70).

Exploratory endpoints include: Change in the ratio of peanut specific IgE to IgG as measured by ImmunoCap assay between placebo and CNP-201 at baseline and at Day 38; Change in peanut specific IgE as measured by ImmunoCap assay between placebo and CNP-201 at baseline and at Day 38.

Preferred embodiments of this disclosure are described herein. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### Example 3—Phase I/II Trial of Low Dose TIMP-PPE in Peanut Allergy

The present example describes a Phase 1b/2a randomized, double-blind, placebo-controlled study to assess the safety, tolerability and pharmacodynamics of TIMP-PPE (CNP-201), in subjects ages 16-55 with peanut allergy.

CNP-201 consists of PLGA nanoparticles encapsulating purified peanut extract having an average diameter of 400-800 nm and a negative zeta potential of between −30 mV and −60 mV. CNP-201 particles are supplied as a lyophilized formulation. CNP-201 particles are reconstituted in sterile water for injection and diluted in sterile saline (0.9% sodium chloride) prior to administration.

The study is a randomized, double-blind, placebo-controlled study of the safety and tolerability of ascending dose levels of CNP-201. The study will enroll 4 cohorts to receive CNP-201 or Placebo at multiple ascending dose levels.

Subjects who meet all inclusion and no exclusion criteria are enrolled into the study. Subjects who continue to meet Inclusion/Exclusion criteria are randomized on Day 1 in a 2:1 ratio to receive either CNP-201 or Placebo (0.9% Sodium Chloride USP) by intravenous (IV) infusion. Subjects are dosed with CNP-201 or Placebo on Day 1 and Day 8.

Subjects may receive pre-administration of an NSAID (such as 325 mg oral acetylsalicylic acid) daily for 2 days prior and 60 minutes prior to each infusion of CNP-201 or Placebo. Subjects may receive pre-administration and/or post-administration of a leukotriene modifier (such as 10 mg oral montelukast) 12 hours prior to, 45 minutes prior to, 12 hour post each infusion of CNP-201 or placebo. Subjects may receive pre-administration of anti-histamine (such as 50 mg IV diphenhydramine) and a corticosteroid (such as 125 mg IV methylprednisone) 30 minutes prior to each infusion of CNP-201 or Placebo.

Subjects will remain on their current SoC during the study, excluding a 12 hour washout period (for beta-agonists, theophylline, and cromolyn only) and a 7 day washout period for antihistamines prior to skin prick test (SPT). Subjects will re-commence their SoC regimen after SPT and remain on SoC through dosing/duration of the study.

Subjects with peanut allergy eligible for enrollment in this study are defined based on the following inclusion criteria:
1. Men and non-pregnant women, ages 16 to 55 years inclusive.
2. Subjects with physician-diagnosed peanut allergy or documented history of peanut allergy.
3. Subjects with weight 31.25 kg at Screening. Subjects who fall outside of this range may be included at the discretion of the investigator.
4. Subjects with a documented history of non-severe anaphylaxis (Grade, â§ 3) to peanuts, including mild wheezing or dyspnea without hypoxia.
5. Subjects with peanut specific IgE≥5 kU/L as measured by ImmunoCAP at Screening unless previously on OIT for their peanut allergy. Subjects who have previously been on OIT for peanut allergy and who do not have peanut specific IgE≥5 kU/L as measured by ImmunoCap at Screening may be included at the discretion of the inestigator, OR subjects with a positive SPT to peanut with a change in wheal diameter≥3 mm as compared to a negative control (50% glycerin) at Screening. Subjects who have previously been on OIT for peanut allergy and who do not have a positive skin prick test (SPT) to peanut with a change in wheal diameter≥3 mm at Screening may be included at the discretion of the investigator.
6. Subjects who are self-reported to be on a peanut free diet with no suspected peanut exposure, including any peanut food challenge, for at least 14 days prior to Screening and agreement to continue restriction to peanut exposure during the study.
7. Subjects with a positive skin prick test (SPT) to peanut with a change in wheal diameter>3 mm as compared to a negative control (50% glycerin) at Screening.
8. Female subjects and male subjects and their female spouse/partners who are willing to practice a highly effective method of contraception that may include, but is not limited to, abstinence, sex only with persons of the same sex, monogamous relationship with vasectomized partner, vasectomy, hysterectomy, bilateral tubal ligation, licensed hormonal methods, intrauterine device (IUD), or use of spermicide combined with a barrier method (e.g., condom, diaphragm) starting at Screening and continuing throughout the entire study to Day 38 (EOS/ET).
9. Female subjects who agree to not breastfeed starting at initial Screening and throughout the entire study to Day 38 (EOS/ET).
10. Female subjects who agree to not donate ova starting at initial Screening and throughout the entire study to Day 38 (EOS/ET).
11. Subjects who are willing and able to provide Institutional Review Board (IRB) approved written informed consent.
12. Subjects who are willing to perform and comply with all study procedures.
13. Male subjects who agree to not donate sperm starting at Screening and throughout the entire study to Day 38 (EOS/ET).

Subjects who meet all inclusion and no exclusion criteria after completing the Screening visit are enrolled into one of the multiple dosing cohorts. Subjects are randomized in a 2:1 ratio to receive either CNP-201 or Placebo (0.9% sodium chloride injection) as a 200 mL intravenous infusion on Day 1 and Day 8. The dose level for the first cohort is 25 mg. Additional dose levels for remaining 3 cohorts are descending to 0.1 mg, 0.25 mg, 0.5 mg, 1 mg, 2 mg, 2.5 mg, 5 mg, 10 mg or maybe ascending up to 650 mg. Dosing of subjects within a dose cohort are separated by at least 48 hours.

Subjects who meet all inclusion and no exclusion criteria at Screening are enrolled into the study. Subjects will return to the clinic on Day 1 for final assessment of eligibility and collection of laboratory samples. Subjects who continue to meet all inclusion and no exclusion criteria are randomized into the dose Cohort open at that time. Subjects may receive pre-administration of an NSAID (such as 325 mg oral acetylsalicylic acid) daily for 2 days prior and 60 minutes prior to each infusion of CNP-201 or Placebo on Day 1 and on Day 8. Subjects may receive pre-administration and/or post-administration of a leukotriene modifier (such as 10 mg oral montelukast) 12 hours prior to, 45 minutes prior to, 12 hour post each infusion of CNP-201 and placebo on Day 1 and Day 8. Subjects may receive pre-administration of antihistamine (such as 50 mg IV diphenhydramine) and a corticosteroid (such as 125 mg IV methylprednisone) 30 minutes prior to each infusion of CNP-201 or Placebo on Day 1 and Day 8.

CNP-201 or Placebo are administered by intravenous infusion over approximately 3-4 hours using a graduated rate of infusion. Subjects undergo medical observation in the clinic for acute adverse events (AEs) including infusion reactions (IR) for 4 hours following the infusion. Antihistamines/epinephrine, including intramuscular (IM) and intravenous (IV) epinephrine, are immediately available to treat allergic reactions, should they occur.

Subjects are followed daily through telephone visits following each infusion (Days 2-7 and Days 9-14) to assess and document any AEs and medication changes. In the Post-Dosing period, subjects return to the clinic 7 days after administration of Dose 2 of CNP-201 or Placebo (Day 15) for collection of safety labs, PD measurements, and assessment of AEs and medication changes.

After all subjects in a dose Cohort have completed the Day 15 office visit (7 days post-second dose), the DMC is convened to review all available safety data and determine whether it is acceptable to proceed to the next ascending dose Cohort.

The subject returns to the clinic for the end of study visit on Day 38 for collection of safety labs and PD measurements, assessment of AEs and medication changes and a second SPT. On Day 60, subject returns to the clinic for safety labs, PD measurements and to undergo a DBPCFC. On Day 90, and 120 subjects will optionally return to the clinic for collection of safety labs and PD measurements. Subjects will optionally return on day 180 for a second DBPCFC consisting of peanut and placebo (oat) challenges, safety labs, PD measurements, and final assessment of AEs and medication changes.

Emerging safety and tolerability data is to be monitored continually. The Medical Monitor is notified of any serious adverse event (SAE) and any, ≥Grade 2 adverse event likely related to test product (CTCAE v.5.0 or CoFAR V.3.0) within 24 hours of becoming aware of such an event. The Medical Monitor may convene an ad-hoc DMC meeting to evaluate safety and tolerability data to determine if it remains acceptable to continue dosing and make recommendations including, but not limited to, continuation of dosing and stopping or pausing dosing in the subject. The DMC may at any point during the study recommend stopping or pausing the study if, in their opinion, continuation of dosing poses an unacceptable safety risk to subjects.

Subjects receive CNP-201 via intravenous infusion lasting approximately 3-4 hours according to the following graduated infusion rate: 1 mL/hr for the first 10 minutes, 2 mL/hr for the next 10 minutes, 5 mL/hr for the next 10 minutes, 10 mL/hr for the next 10 minutes, 20 mL/hr for the next 15 minutes, 40 mL/hr for the next 15 minutes, 80 mL/hr for the remainder of the infusion.

Study duration: 2 doses, 7 days apart. The total duration of the study for an individual subject is ~67 days; 7 days for Screening; 8 days for Test Product dosing; and 52 days for post-dosing evaluation. An additional 120 day post-dosing evaluation is optional.

Primary objectives include: Safety and tolerability of CNP-201.

Exploratory objectives include: Change in response to a DBPCFC among patients treated with CNP-201 or placebo, change in the ratio of peanut specific IgE to IgG among patients treated with CNP-201 or placebo, change in peanut specific IgE among patients treated with CNP-201 or placebo, change in the proportion of peanut specific Th2a+ T cells among patients treated with CNP-201 or placebo, change in the response to a Basophil Activation Test among patients treated with CNP-201 or placebo, change in the proportion of peanut specific T regulatory cells among patients treated with CNP-201 or placebo.

Primary endpoints include: Frequency of Adverse Events (AEs) and Serious Adverse Events (SAEs), MedDRA 23.0 (CTCAE v.5.0 or CoFAR V.3.0 for allergy related AEs); Laboratory Safety Assessments (hematology, serum chemistry, coagulation panel, and urinalysis); Physical Examinations including vital signs (blood pressure, heart rate, and temperature); 12-lead electrocardiogram (ECG); Serum Cytokines (TNF-α, IL-2, IL-6, IL-8, IL-1β, MCP-1, MIP-1β, MIP-1α, IFN-γ, and IL-12p70).

Exploratory endpoints include: difference in percentage of subjects who pass a DBPCFC (do not reach an eliciting dose at or before the 2000 mg dose level, 4043 mg cumulative) between placebo and CNP-201 at Day 60 and Day 180, change in the cumulative tolerated dose (CTD) of peanut protein (mg) administered during a DBPCFC between placebo and CNP-201 at Day 60 and Day 180, change in the ratio of peanut specific IgE to IgG as measured by ImmunoCap assay between placebo and CNP-201 at baseline and at Day 38, change in peanut specific IgE as measured by ImmunoCap assay between placebo and CNP-201 at baseline and at Day 38, change in the proportion of peanut specific Th2a+ T cells (peanut specific Th2a+ cells/total peanut specific T cells) following ex vivo stimulation of PBMCs between placebo and CNP-201 at baseline and at Day 15, change in the effective concentration at 50% of maximal basophil activation (EC50) as measured by a Basophil Activation Test (CD203c+/CD63+/− basophil activation) between placebo and CNP-201 at baseline and at Day 60 and Day 180, change in the proportion of peanut specific T regulatory cells (peanut specific T regulatory cells/peanut specific CD4+ effector memory cells) following ex vivo stimulation of PBMCs between placebo and CNP-201 at baseline and at Day 15.

### Example 4—Interim Results from a Phase I/II Trial of Low Dose TIMP-PPE in Peanut Allergy

The present example describes results from a Phase 1b/2a randomized, double-blind, placebo-controlled study demonstrating that administration of low doses of CNP-201 is efficacious in treating peanut allergy.

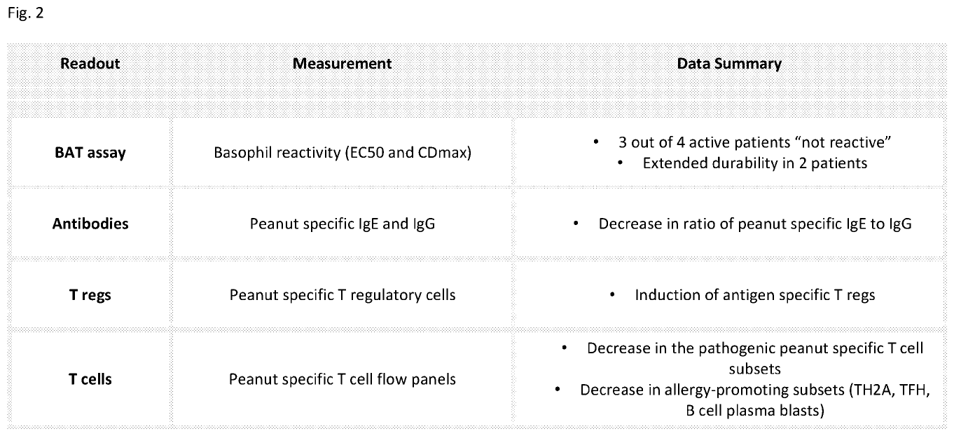

Subjects received a single intravenous dose of either 1 mg or 25 mg CNP-201 on day 1. FIG. 2 summarizes results for subjects with peanut allergy receiving low dose CNP-201 particles in peanut allergy. 3 out of 4 patients receiving low dose CNP-201 had increased BAT thresholds with extended durability in 2 patients up to day 60. Peanut allergic patients receiving up to 25 mg dose of CNP-201 showed a decrease in the ratio of peanut specific IgE to IgG at day 15 and day 38. Peanut allergic patients receiving one 25 mg dose of CNP-201 showed an induction of antigen specific Tregs and a decrease in pathogenic peanut specific T cell subsets (Th2a, TFH, B cell plasma blasts).

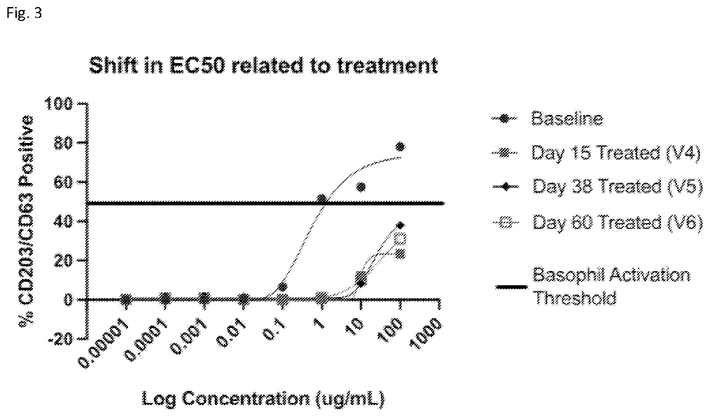

For the BAT test blood was drawn before and after low-dose CNP-201 treatment. PBMCs isolated from blood were stimulated with increasing concentration of peanut allergen. Basophil degranulation was measured by CD203 and CD63 positive marker expression on basophils by flow cytometry. The activation threshold i.e. cutoff for positive response is 50% CD203⁺CD63⁺ expression. In FIG. 3, basophil activation was detected before treatment with 25 mg CNP-201. Peanut allergic subject receiving one 25 mg intravenous dose of CNP-201 showed an increase in the EC50 (concentration of peanut allergen) needed to increase expression of activation markers on the surface of basophils following stimulation with allergen. Post treatment with CNP-201, basophil degranulation was lower than the activation threshold at days 15, 38 and 60. The increase in BAT demonstrates non-reactivity to peanut allergen.

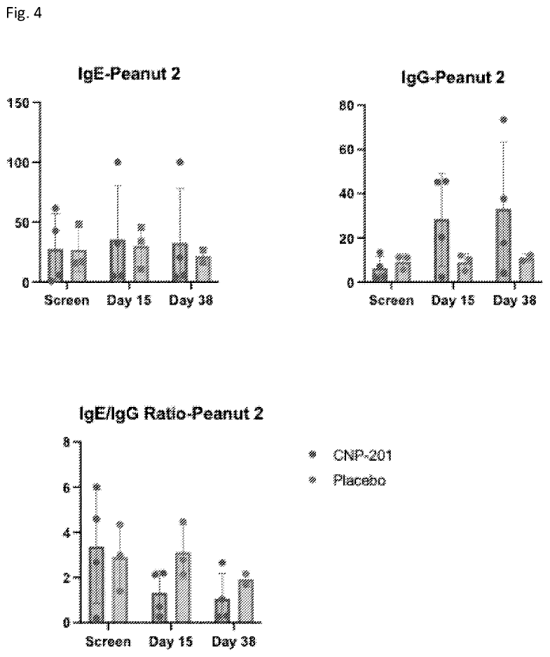

Peanut specific IgE and IgG levels were determined from patient sera. Peanut allergic patients receiving up to 25 mg intravenous dose of CNP-201 showed an increase in peanut specific IgG while the peanut specific IgE levels did not change (FIG. 4). A decrease in peanut specific IgE/IgG ratio was detected at day 15 and day 38 after dosing compared to placebo (FIG. 4).

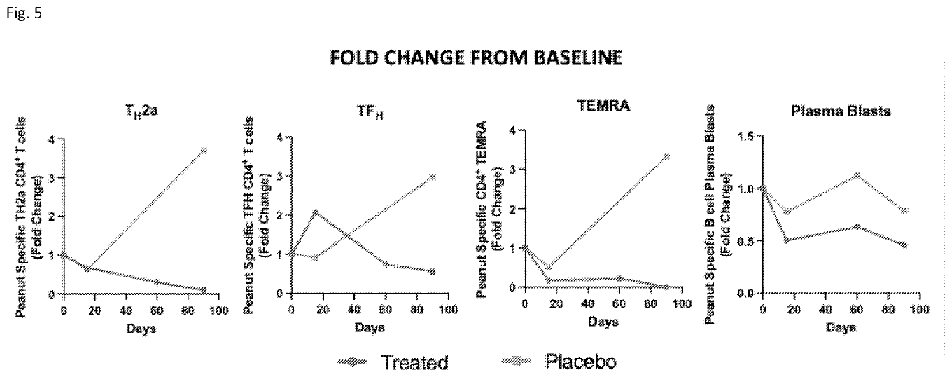

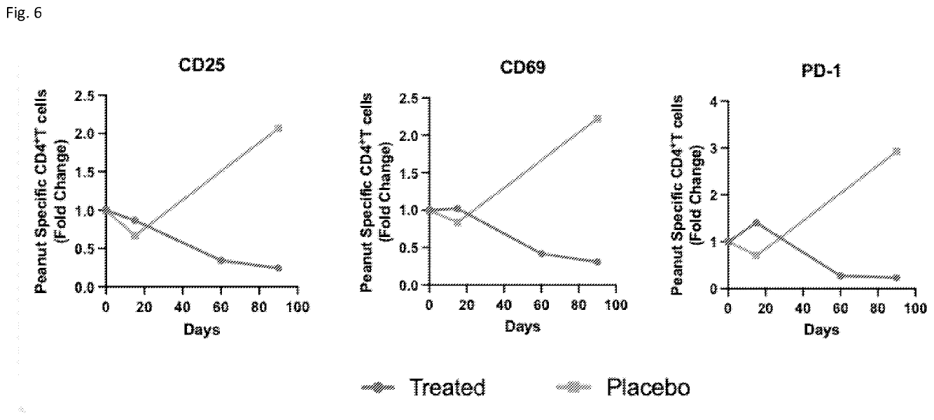

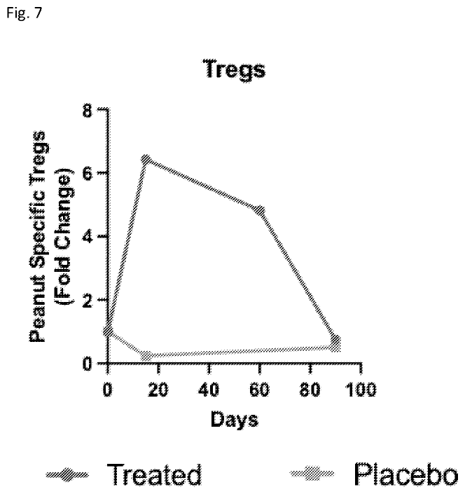

PBMCs obtained from blood of a peanut allergic patient who had received a single dose of 25 mg CNP-201 were stimulated with whole peanut extract and CD40 ligand. Subpopulations of peanut specific activated T cells were further characterized by expression of lineage specific markers. FIG. 5 shows reduced peanut allergy associated immune cell subsets up to 100 days after administration of a single 25 mg dose of CNP-201. Peanut allergic patient receiving a single 25 mg intravenous dose of CNP-201 showed a decrease in allergy-promoting subsets T helper 2 (Th2A), T follicular helper cell (TFH), terminally differentiated effector memory cells (TEMRA), and B cell plasma blasts. FIG. 6 shows lower pathogenic activated peanut specific CD4⁺ T cells after administration of 25 mg CNP-201. There was a decrease in the peanut specific activated T cell subsets CD4⁺CD25⁺, CD4⁺CD69⁺ and CD4⁺PD-1⁺ compared to placebo in the low dose CNP-201 treated patient. Peanut allergic subject receiving one 25 mg intravenous dose of CNP-201 showed an increase in peanut specific Tregs (CD4⁺CD137⁺CD25⁺CD127^{lo}) compared to placebo (FIG. 7).

### Example 5: Phase I/II Trial of 1 mg Dose TIMP-PPE is Peanut Allergy

The present example describes a Phase 1b/2a randomized, double-blind, placebo-controlled study to assess the safety, tolerability and pharmacodynamics of TIMP-PPE (CNP-201), in subjects ages 16-55 with peanut allergy.

CNP-201 consists of PLGA nanoparticles encapsulating purified peanut extract having an average diameter of 400-800 nm and a negative zeta potential of between −30 mV and −80 mV. CNP-201 particles are supplied as a lyophilized formulation. CNP-201 particles are reconstituted in sterile water for injection and diluted in sterile saline (0.9% sodium chloride) prior to administration.

The study is a randomized, double-blind, placebo-controlled study of the safety, pharmacodynamics, and tolerability of multiple ascending dose levels of CNP-201 (Escalation Phase) with the goal of identifying a safe and tolerable dose level to be evaluated further in a larger number of subjects (Expansion Phase). The study will enroll 4 cohorts to receive CNP-201 or Placebo at multiple ascending dose levels.

Subjects will remain on their current SoC during the study, excluding a 12 hour washout period (for beta-agonists, theophylline, and cromolyn only) and a 7 day washout period for antihistamines prior to skin prick test (SPT). Subjects will re-commence their SoC regimen after SPT and remain on SoC through dosing/duration of the study.

Subjects with peanut allergy eligible for enrollment in this study are defined based on the following inclusion criteria:
1. Men and non-pregnant women, ages 16 to 55 years inclusive.
2. Subjects with physician-diagnosed peanut allergy or documented history of peanut allergy.
3. Subjects with weight≥31.25 kg at Screening. Subjects who fall outside of this range may be included at the discretion of the investigator.
4. Subjects with a documented history of non-severe anaphylaxis (Grade≤3) to peanuts, including mild wheezing or dyspnea without hypoxia.
5. Subjects with peanut specific IgE≥5 kU/L as measured by ImmunoCAP at Screening unless previously on OIT for their peanut allergy. Subjects who have previously been on OIT for peanut allergy and who do not have peanut specific IgE≥5 kU/L as measured by ImmunoCap at Screening may be included at the discretion of the investigator.
6. Subjects with a positive SPT to peanut with a change in wheal diameter≥5 mm as compared to a negative control (50% glycerin) at Screening. Subjects who have previously been on OIT for peanut allergy and who do not have a positive skin prick test (SPT) to peanut with a change in wheal diameter≥5 mm at Screening may be included at the discretion of the investigator.
7. Subjects who are self-reported to be on a peanut free diet with no suspected peanut exposure, including any peanut food challenge, for at least 14 days prior to Screening and agreement to continue restriction to peanut exposure during the study.
8. Female subjects and male subjects and their female spouse/partners who are willing to practice a highly effective method of contraception that may include, but is not limited to, abstinence, sex only with persons of the same sex, monogamous relationship with vasectomized partner, vasectomy, hysterectomy, bilateral tubal ligation, licensed hormonal methods, intrauterine device (IUD), or use of spermicide combined with a barrier method (e.g., condom, diaphragm) starting at Screening and continuing throughout the entire study to Day 38 (EOS/ET).
9. Female subjects who agree to not breastfeed starting at initial Screening and continuing to Day 60.
10. Female subjects who agree to not donate ova starting at initial Screening and continuing to Day 60.
11. Subjects who are willing and able to provide Institutional Review Board (IRB) approved written informed consent.
12. Subjects who are willing to perform and comply with all study procedures.
13. Male subjects who agree to not donate sperm starting at Screening and continuing to Day 60.

Subjects who meet all inclusion and no exclusion criteria after completing the Screening visit are enrolled into one of the multiple dosing cohorts. In the Escalation Phase, subjects will receive CNP-201 as an intravenous infusion on Day 1 and Day 8. The dose level for the first cohort is 1 mg. Additional dose levels for remaining 3 cohorts are descending to 0.1 mg, 0.25 mg, 0.5 mg, 1 mg, 2 mg, 2.5 mg, 5 mg, 10 mg or can be ascending up to 650 mg.

Subjects who meet all inclusion and no exclusion criteria at Screening are enrolled into the study. CNP-201 or Placebo are administered by intravenous infusion over using a graduated rate of infusion. CNP-201 will be administered using a graduated rate of infusion as follows:1 mL/hr for the first 10 min, 2 mL/hr for the next 10 min, 5 mL/hr for the next 10 min, 10 mL/hr for the next 10 min, 20 mL/hr for the remainder of the infusion.

Subjects remain in the clinic on Day 1 and Day 8 from the time of admission (prior to administration of TP) through the final procedure conducted 4 hours post-dose that same day unless an infusion reaction, anaphylaxis, or other adverse event requires an extended duration of monitoring. Subjects are discharged if safety parameters are acceptable to the investigator.

Subjects are followed daily through telephone visits following each infusion (Days 2-7 and Days 9-14) to assess and document any AEs and medication changes.

Seven days after the second administration of CNP-201 (Day 15), subjects must return to the clinic for collection of safety labs, assessment of AEs and medication changes, and collection of PD assessments. Subjects continue to be followed for safety and tolerability during the Post-Dosing period.

On Day 60 (End of Study), subjects return to the clinic for collection of immune safety labs, PD measurements and to undergo a Double-Blind, Placebo-Controlled Food Challenge (DBPCFC) consisting of peanut and placebo (oat) challenges. The DBPCFC is conducted with a study physician or staff trained to manage clinical emergencies present on site, with immediate access to emergency medications and equipment, and within close proximity to hospital emergency departments for rapid delivery of urgent care if needed.

Subjects then return to the clinic for the end of study visit on Day 60 for collection of safety labs, PD measurements, and final assessment of AEs and medication changes.

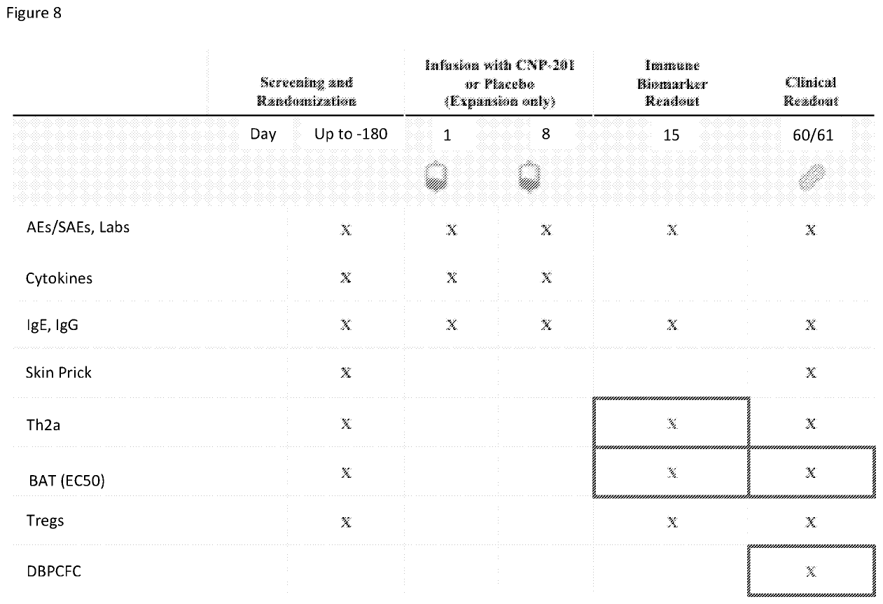

The total duration of the study for an individual subject is ~240 days; up to 180 days for Screening, 8 days for CNP-201 or placebo dosing and 52 days post-dosing evaluation (FIG. 8).

After all subjects in a dose Cohort have completed Day 15 office visit (7 days post-second dose), the DMC is convened to review all available safety data. At this time, the DMC determines whether it is acceptable to proceed to the next dose Cohort and to make a recommendation on the dose level for that Cohort.

If a safe and effective dose is identified in the Escalation Phase, the study proceeds to the Expansion Phase. Subjects in the Expansion Phase are randomized in a 1:1 ratio to receive either a safe and tolerable dose level of CNP-201 identified in the Escalation Phase or Placebo. Subjects in the Escalation and Expansion Phases will undergo the same assessments.

Primary objectives include: Safety and tolerability of CNP-201.

Exploratory objectives include: Change in response to a DBPCFC among patients treated with CNP-201 or placebo, change in the ratio of peanut specific IgE to IgG among patients treated with CNP-201 or placebo, change in peanut specific IgE among patients treated with CNP-201 or placebo, change in the proportion of peanut specific Th2a+ T cells among patients treated with CNP-201 or placebo, change in the response to a Basophil Activation Test among patients treated with CNP-201 or placebo, change in the proportion of peanut specific T regulatory cells among patients treated with CNP-201 or placebo.

Primary endpoints include: Frequency of Adverse Events (AEs) and Serious Adverse Events (SAEs), MedDRA 23.0 (CTCAE v.5.0 or CoFAR V.3.0 for allergy related AEs); Laboratory Safety Assessments (hematology, serum chemistry, coagulation panel, and urinalysis); Physical Examinations including vital signs (blood pressure, heart rate, and temperature); 12-lead electrocardiogram (ECG); Serum Cytokines (TNF-α, IL-2, IL-6, IL-8, IL-1β, MCP-1, MIP-1β, MIP-1α, IFN-γ, and IL-12p70).

Exploratory endpoints include: difference in percentage of subjects who pass a DBPCFC (do not reach an eliciting dose at or before the 2000 mg dose level, 4043 mg cumulative) between placebo and CNP-201 at Day 60, change in the cumulative tolerated dose (CTD) of peanut protein (mg) administered during a DBPCFC between placebo and CNP-201 at Day 60, change in the ratio of peanut specific IgE to IgG as measured by ImmunoCap assay between placebo and CNP-201 at baseline and at Day 60, change in peanut specific IgE as measured by ImmunoCap assay between placebo and CNP-201 at baseline and at Day 60, change in the proportion of peanut specific Th2a+ T cells (peanut specific Th2a+ cells/total peanut specific T cells) following ex vivo stimulation of PBMCs between placebo and CNP-201 at baseline and at Day 15, change in the effective concentration at 50% of maximal basophil activation (EC50) as measured by a Basophil Activation Test (CD203c+/CD63+/− basophil activation) between placebo and CNP-201 at baseline and at Day 15 and Day 60, change in the proportion of peanut specific T regulatory cells (peanut specific T regulatory cells/peanut specific CD4+ effector memory cells) following ex vivo stimulation of PBMCs between placebo and CNP-201 at baseline and at Day 15.

Preferred embodiments of this disclosure are described herein. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method of treating peanut allergy in a subject comprising administering to the subject tolerizing immune modifying particles encapsulating peanut proteins (TIMP-PPE), alone or in combination with one or more therapeutics, wherein TIMP-PPE is administered at a dose level of between about 0.001 mg/kg and 12 mg/kg.

2. The method of claim 1, wherein the TIMP-PPE particles have an average diameter of between 100 nm and 1500 nm.

3. The method of claim 1, wherein the TIMP-PPE particles have a negative zeta potential.

4. The method of claim 1, wherein the particles have a negative zeta potential of between −30 mV and −100 mV.

5. The method of any one of the preceding claims, wherein TIMP-PPE
i) is administered at a concentration of between about 0.0005 mg/mL and about 50 mg/mL,
ii) is administered at a dose level of about 0.001 mg/kg, 0.0025 mg/kg, 0.005 mg/kg, 0.01 mg/kg, 0.025 mg/kg, 0.05 mg/kg, 0.1 mg/kg, 0.25 mg/kg, 0.5 mg/kg, 1.0 mg/kg, 2.0 mg/kg, 4.0 mg/kg, 6 mg/kg, 8.0 mg/kg, 10 mg/kg, or 12 ma/kg; or
iii) is administered at a dose level of between about 0.1 ma and 800 mg.

6-8. (canceled)

9. The method of claim 1 wherein TIMP-PPE is administered in a single dose or in multiple doses.

10. The method of claim 1, wherein TIMP-PPE is administered once weekly, once every two weeks, once every three weeks, once every 4 weeks, once every two months, once every three months, once every 6 months, or once per year.

11. The method of claim 1, wherein TIMP-PPE is administered in two doses one-week apart or wherein TIMP-PPE is administered in two doses one-week apart followed by a booster dose of TIMP-PPE re-administered as a single dose once every three months.

12. The method of claim 1, wherein TIMP-PPE is administered intravenously, subcutaneously, intramuscularly, intraperitoneally, intranasally, or orally.

13-14. (canceled)

15. The method of claim 1, wherein the therapeutic administered in combination with TIMP-PPE is an anti-IgE antibody, anti-IL-4Rα antibody, anti-IL13 antibody, an anti-IL-33 antibody, an antihistamine, a steroid, a corticosteroid, a leukotriene modifier, low dose IL-2, IL-2 muteins engineered to expand Tregs, IL-2 variants engineered to expand Tregs, IL-2 molecules engineered to be selective for the high-affinity IL-2 receptor, PEGylated IL-2, IL-2 complexes, IL-2/CD25 fusion proteins, a prebiotic, a probiotic, histone deacetylase inhibitor, short chain fatty acids (e.g. acetate, butyrate, propionate, butyrate polymer), an inhibitor of IgE, competitor of IgE for allergen binding sites, an inhibitor of basophil activation, an inhibitor of mast cell activation, a cytokine inhibitor, microbiome therapy, a small molecule or biological therapeutic, or a nonsteroid anti-inflammatory drug (NSAID).

16-24. (canceled)

25. The method of claim 1, wherein administering TIMP-PPE alone or in combination with an additional therapeutic to the subject reduces one or more symptoms of peanut allergy.

26. The method of claim 25, wherein the one or more symptoms of peanut allergy are selected from the group consisting of skin reactions, hives, skin redness, skin swelling, itching, tightening of the throat, difficulty breathing, shortness of breath, digestive problems such as diarrhea, stomach cramps, nausea, or vomiting, drop in blood pressure and anaphylaxis.

27. The method of claim 1, wherein administering TIMP-PPE alone or in combination with a therapeutic to the subject reduces the duration and severity of the allergic immune response to peanut proteins.

28. The method of claim 27, wherein the allergic immune response is a Th2 cell response, B cell activation, basophil activation, eosinophil activation, mast cell activation, and/or IgE induction.

29-30. (canceled)

31. The method of claim 27, wherein administering TIMP-PPE alone or in combination with a therapeutic to the subject
i) decreases the proportion of Th2a+ T cells present in the total T cell population in peripheral blood;
ii) decreases the proportion of activated peanut protein specific T cells in peripheral blood,
iii) increases the levels of peanut protein specific Treg cells in blood,
iv) decreases basophil activation reduces peanut protein specific IgE levels in blood,
v) reduces the levels of Th2 cytokine levels in blood, and/or
vi) increases tolerance to peanut proteins.

32-42. (canceled)

43. The method of claim 1, wherein the TIMP-PPE comprises a peanut extract or one or more peanut proteins or antigenic fragments thereof selected from the group consisting of Ara h1, Ara h2, Ara h3, Ara h5, Ara h6, Ara h7, Ara h8, Ara h9, Ara h10, Ara h11, Ara h12, Ara h13, Ara h14, Ara h15, Ara h16, Ara h17, and Ara h18.

44. The method of claim 1, wherein the TIMP-PPE comprises one or more proteins or antigenic fragments thereof of Ara h1, Ara h2, Ara h3, Ara h5, Ara h6, Ara h7, or Ara h8 or combinations thereof.

45. The method of claim 1, wherein the subject is administered an antihistamine, a corticosteroid, a NSAID and/or a leukotriene modifier in combination with TIMP-PPE.

46-65. (canceled)


SEQUENCE LISTING (25 sequences)

SEQ ID NO: 1 - protein, 619 aa, Arachis hypogaea
MRGRVSPLMLLLGILVLASVSATHAKSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRRALILTRAMHSESHPFHFLHLDDMTPELRVAKSHAVNTPGQFEDFFPASSRDQSSYLQGFSRNTLEAAFNAEFNEIRRVLLEENAGGEQEERGQRRWSTRSSENNEGVIVEVSKEHVEELTKHAKSVSKKGSEEEGDITNPINLREGEPDLSDNFGRLFEVKPDKKNPQLQDLDMMLTCVEIKEGALMLPHFNSKAMVIVVINKGTGNLELVAVRKEQQQRGRREQEWEEEEEDEEEEGSNREVRRYTARLKEGDVFIMPAAHPVAINASSELHLLGFGINAENNHRIFLAGDKDNVVDQIEKQAKDLAFPGSGEQVEKLIKNQRESHFVSARPQSQSPSSPEKEDQEEENQGGKGPLLSILKAFN

SEQ ID NO: 2 - protein, 160 aa, Arachis hypogaea
MAKLTILVALALFLLAAHASARQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGRQQEQQFKRELRNLPQQCGLRAPQRCDLDVESGGRDRY

SEQ ID NO: 3 - protein, 512 aa, Arachis hypogaea
MAKLLALSLCFCVLVLGASSVTFRQGGEENECQFQRLNAQRPDNRIESEGGYIETWNPNNQEFQCAGVALSRTVLRRNALRRPFYSNAPLEIYVQQGSGYFGLIFPGCPSTYEEPAQEGRRYQSQKPSRRFQVGQDDPSQQQQDSHQKVHRFDEGDLIAVPTGVAFWMYNDEDTDVVTVTLSDTSSIHNQLDQFPRRFYLAGNQEQEFLRYQQQQGSRPHYRQISPRVRGDEQENEGSNIFSGFAQEFLQHAFQVDRQTVENLRGENEREEQGAIVTVKGGLRILSPDEEDESSRSPPNRREEFDEDRSRPQQRGKYDENRRGYKNGIEETICSASVKKNLGRSSNPDIYNPQAGSLRSVNELDLPILGWLGLSAQHGTIYRNAMFVPHYTLNAHTIVVALNGRAHVQVVDSNGNRVYDEELQEGHVLVVPQNFAVAAKAQSENYEYLAFKTDSRPSIANQAGENSIIDNLPEEVVANSYRLPREQARQLKNNNPFKFFVPPFDHQSMREVA

SEQ ID NO: 4 - protein, 507 aa, Arachis hypogaea
RQQPEENACQFQRLNAQRPDNRIESEGGYIETWNPNNQEFECAGVALSRLVLRRNALRRPFYSNAPQEIFIQQGRGYFGLIFPGCPRHYEEPHTQGRRSQSQRPPRRLQGEDQSQQQRDSHQKVHRFDEGDLIAVPTGVAFWLYNDHDTDVVAVSLTDTNNNDNQLDQFPRRFNLAGNTEQEFLRYQQQSRQSRRRSLPYSPYSPQSQPRQEEREFSPRGQHSRRERAGQEEENEGGNIFSGFTPEFLEQAFQVDDRQIVQNLRGETESEEEGAIVTVRGGLRILSPDRKRRADEEEEYDEDEYEYDEEDRRRGRGSRGRGNGIEETICTASAKKNIGRNRSPDIYNPQAGSLKTANDLNLLILRWLGPSAEYGNLYRNALFVAHYNTNAHSIIYRLRGRAHVQVVDSNGNRVYDEELQEGHVLVVPQNFAVAGKSQSENFEYVAFKTDSRPSIANLAGENSVIDNLPEEVVANSYGLQREQARQLKNNNPFKFFVPPSQQSPRAVA

SEQ ID NO: 5 - protein, 530 aa, Arachis hypogaea
MAKLLELSFCFCFLVLGASSISFRQQPEENACQFQRLNAQRPDNRIESEGGYIETWNPNNQEFECAGVALSRLVLRRNALRRPFYSNAPQEIFIQQGRGYFGLIFPGCPSTYEEPAQQGRRYQSQRPPRRLQEEDQSQQQQDSHQKVHRFNEGDLIAVPTGVAFWLYNDHDTDVVAVSLTDTNNNDNQLDQFPRRFNLAGNHEQEFLRYQQQSRQSRRRSLPYSPYSPHSRPRREEREFRPRGQHSRRERAGQEEEDEGGNIFSGFTPEFLEQAFQVDDRQIVQNLWGENESEEEGAIVTVRGGLRILSPDGTRGADEEEEYDEDQYEYHEQDGRRGRGSRGGGNGIEETICTACVKKNIGGNRSPHIYDPQRWFTQNCHDLNLLILRWLGLSAEYGNLYRNALFVPHYNTNAHSIIYALRGRAHVQVVDSNGNRVYDEELQEGHVLVVPQNFAVAGKSQSENFEYVAFKTDSRPSIANFAGENSFIDNLPEEVVANSYGLPREQARQLKNNNPFKFFVPPFQQSPRAVA

SEQ ID NO: 6 - protein, 131 aa, Arachis hypogaea
MSWQTYVDDHLLCEIEGNHLSSAAILGQDGSVWAQSSNFPQFKPEEITAIMNDFAEPGSLAPTGLYLGGTKYMVIQGEPGTVIRGKKGPGGVTIKKTNQALIIGIYDEPMTPGQCNMIVEKLGDYLIDTGL

SEQ ID NO: 7 - protein, 145 aa, Arachis hypogaea
MAKSTILVALLALVLVAHASAMRRERGRQGDSSSCERQVDRVNLKPCEQHIMQRIMGEQEQYDSYDIRSTRSSDQQQRCCDELNEMENTQRCMCEALQQIMENQCDRLQDRQMVQQFKRELMNLPQQCNFRAPQRCDLDVSGGRC

SEQ ID NO: 8 - protein, 164 aa, Arachis hypogaea
MVKLSILVALLGALLVVASATRWDPDRGSRGSRWDAPSRGDDQCQRQLQRANLRPCEEHIRQRVEKEQEQEQDEYPYIQRGSRGQRPGESDEDQEQRCCNELNRFQNNQRCMCQALQQILQNQSFRFQQDRSQLHQMERELRNLPQNCGFRSPSRCDLSSRTPY

SEQ ID NO: 9 - protein, 157 aa, Arachis hypogaea
MGVFTFEDEITSTVPPAKLYNAMKDADSITPKIIDDVKSVEIVEGNGGPGTIKKLTIVEDGETKFILHKVESIDEANYAYNYSVVGGVALPPTAEKITFETKLVEGPNGGSIGKLTLKYHTKGDAKPDEEELKKGKAKGEGLFRAIEGYVLANPTQY

SEQ ID NO: 10 - protein, 116 aa, Arachis hypogaea
MASLKFAFVMLVCMAMVGAPMVNAISCGQVNSALAPCIPFLTKGGAPPPACCSGVRGLLGALRTTADRQAACNCLKAAAGSLRGLNQGNAAALPGRCGVSIPYKISTSTNCATIKF

SEQ ID NO: 11 - protein, 92 aa, Arachis hypogaea
LSCGQVNSALAPCITFLTKGGVPSGPCCSGVRGLLGAAKTTADRQAACNCLKAAAGSLHGLNQGNAAALPGRCGVSIPYKISTSTNCATIKF

SEQ ID NO: 12 - protein, 169 aa, Arachis hypogaea
MTDRTQPHTVQVHTTAGRFGDTAAGTNRYPDRGPSTSKVIAVITGLPIGGTLLLFAGLALAGTLLGLAVTTPLFILFSPVIVPAIIVVGLSVAGFLTSGACGLTGLSSFSWVMNYIRQTHGSVPEQLEMAKHRMADVAGYVGQKTKDVGQKTKEVGQEIQTKAQDSKRT

SEQ ID NO: 13 - protein, 150 aa, Arachis hypogaea
MTDRTQPHAVQVHTTAGRFGDTAAGTNRYADRGPSTSKVIAVITGLPIGGTLLLFAGLALAGTLLGLAVTTPLFILFSPVIVPATIVVGLSVAGFLTSGACGLTGLSSFSWVMNYIRQTHGSVPEQLEMAKHRMADVAGYVGQKTKDVGQ

SEQ ID NO: 14 - protein, 137 aa, Arachis hypogaea
MAEALYYGGRQRQEQPRSTQLVKATTAVVAGGSLLILAGLVLAGTVIGLTTITPLFVIFSPVLVPAVITVALLGLGFLASGGFGVAAITVLTWIYRYVTGKHPPGANQLDTARHKLMGKAREIKDFGQQQTSGAQAS

SEQ ID NO: 15 - protein, 137 aa, Arachis hypogaea
MAEALYYGGRQRQDQPRSTQLVKATTAVVAGGSLLILAGLVLAATVIGLTTITPLFVIFSPVLVPAVITVALLGLGFLASGGFGVAAITVLTWIYRYVTGKHPPGANQLDTARHKLMSKAREIKDYGQQQTSGAQAS

SEQ ID NO: 16 - protein, 71 aa, Arachis hypogaea
KTVAGFCIFFLVLFLAQEGVVKTEAKLCNHLADTYRGPCFTNASCDDHCKNKEHFVSGTCMKMACWCAHNC

SEQ ID NO: 17 - protein, 79 aa, Arachis hypogaea
VQKRTIIMEKKMAGFCIFFLILFLAQEYGVEGKECLNLSDKFKGPCLGSKNCDHHCRDIEHLLSGVCRDDFRCWCNRKC

SEQ ID NO: 18 - protein, 72 aa, Arachis hypogaea
MEKKMAGFCIFFLVLFLAQEYGVEGKVCLNLSDKFKGPCLGTKNCDHHCRDIEHLLSGVCRDDFRCWCNRNC

SEQ ID NO: 19 - protein, 176 aa, Arachis hypogaea
MATATDRAPHQVQVHTPTTQRVDVPRRGYDVSGGGIKTLLPERGPSTSQIIAVLVGVPTGGTLLLLSGLSLLGTIIGLAIATPVFTFFSPVIVPAVVTIGLAVTGILTAGACGLTGLMSLSWMINFIRQVHGTTVPDQLDSVKRRMADMADYVGQKTKDAGQQIQTKAQDVKRSSS

SEQ ID NO: 20 - protein, 176 aa, Arachis hypogaea
MATATDRAPHQVQVHTPTTQRVDVQRRGYDVSGGGVKTFLPDRGPSTSQIIAVLVGVPTGGTLLLLSGLSLLGTIIGLAIATPVFTFFSPVIVPAVVTIGLAVIGILTAGACGLTGLMSLSWMINFIRQVHGTTVPDQLDSAKRRMADMADYVGQKTKDAGQEIQTKAQDVKRSSS

SEQ ID NO: 21 - protein, 176 aa, Arachis hypogaea
MATATDRAPHQVQVHTPTTQRVDVPRRGYDVSGGGIKTLLPERGPSTSQIIAVLVGVPTGGTLLLLSGLSLLGTIIGLAIATPVFIFFSPVIVPAVVTIGLAVTGILTAGACGLTGLMSLSWMINFIRQVHGTTVPDQLDSVKRRMADMADYVGQKTKDAGQEIQTKAQDVKRSSS

SEQ ID NO: 22 - protein, 166 aa, Arachis hypogaea
MSDQTRTGYGGGGSYGSSYGGGGTYGSSYGTSYDPSTNQPIRQAIKFMTASTIGVSFLILSGLILTGTVIGLIIATPLLVIFSPILVPAAITLALAAGGFLFSGGCGVAAIAALSWLYSYVTGKHPAGSDRLDYAKGVIADKARDVKDRAKDYAGAGRAQEGTPGY

SEQ ID NO: 23 - protein, 68 aa, Arachis hypogaea
VTCTPTELSPCLGAITGGSPPSSVCCQKLRAQKPCLCNYIKNPALRTYVNSPGARRVASSCGVPLPSC

SEQ ID NO: 24 - protein, 93 aa, Arachis hypogaea
AIQCSFVTKSIAPCFGYLKSGGTVSGPCCSGIQNINGTAKTTSDRQAVCNCLKSAAASLGSQINPNNAASLPGKCGVSIPYKISTSINCSSIK

SEQ ID NO: 25 - protein, 226 aa, Arachis hypogaea
MANPRVFFDMTIGGQPAGRIIMELFADTTPRTAENFRALCTGEKGVGRSGKPLHYKGSSFHRVIPNFMCQGGDFTAGNGTGGESIYGSKFADENFIKKHTGPGILSMANAGQNTNGSQFFICTAKTEWLDGKHVVFGQVVEGLDVVREIEKVGSSSGRTSKPVVVADCGQLCNAKLTMSWGNGAYRKSLQVQGVVIGQRDANSMSEQAQFAFVVSFVTQIMKIQSS